SEQUENCE LISTING (94 sequences)

SEQ ID NO: 1 - protein, 214 aa, synthetic construct
DIQMTQSPSSLSASVGDRVTITCRASQGVGTNVGWYQQKPGKAPKALIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, synthetic construct
EVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, synthetic construct
DIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 214 aa, synthetic construct
DIQMTQSPSSLSASVGDRVTITCRASQGVGTNVGWYQQKPGKAPKLLIYSASFLYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 218 aa, synthetic construct
DIVMTQSPDSLAVSLGERATINCKASESVDTSDNSFIHWYQQKPGQSPKLLIYRASTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 218 aa, synthetic construct
DIVMTQSPDSLAVSLGERATINCKASESVDTSDNSFIHWYQQKPGQSPKLLIYRESTQESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 218 aa, synthetic construct
DIVMTQSPDSLAVSLGERATINCKASESVDTSDNSFIHWYQQKPGQSPKLLIYRASTQESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, synthetic construct
EVQLVESGGGLVQPGGSLRLSCAASGFTFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 449 aa, synthetic construct
EVQLVESGGGLVQPGGSLRLSCAASGFTFSSYAMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 449 aa, synthetic construct
EVQLLESGGGLVQPGGSLRLSCAASGFSFSSYTMSWVRQAPGKGLEWVATISGGGRDLYYPESVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 449 aa, synthetic construct
EVQLLESGGGLVQPGGSLRLSCAASGFSFSSYTMSWVRQAPGKGLEWVATISGGGRDTYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 449 aa, synthetic construct
EVQLLESGGGLVQPGGSLRLSCAASGFSFSSYTMSWVRQAPGKGLEWVATISGGGRDTYYADSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 449 aa, synthetic construct
EVQLLESGGGLVQPGGSLRLSCAASGFSFSSYTMSWVRQAPGKGLEWVATISGGGRDIYYADSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 449 aa, synthetic construct
EVQLLESGGGLVQPGGSLRLSCAASGFSFSSYTMSWVRQAPGKGLEWVATISGGGRDIYFPESVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 449 aa, synthetic construct
EVQLLESGGGLVQPGGSLRLSCAASGFSFSSYTMSWVRQAPGKGLEWVSAISGGGRDLYYPESVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 449 aa, synthetic construct
EVQLLESGGGLVQPGGSLRLSCAASGFSFSSYTMSWVRQAPGKGLEWVSAISGGGRDTYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 449 aa, synthetic construct
EVQLLESGGGLVQPGGSLRLSCAASGFSFSSYTMSWVRQAPGKGLEWVSAISGGGRDTYYADSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 449 aa, synthetic construct
EVQLLESGGGLVQPGGSLRLSCAASGFSFSSYTMSWVRQAPGKGLEWVSAISGGGRDIYYADSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 449 aa, synthetic construct
EVQLLESGGGLVQPGGSLRLSCAASGFSFSSYTMSWVRQAPGKGLEWVSAISGGGRDIYFPESVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 449 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 23 - protein, 879 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGPQARKGGSGGGGSGGGGSGGGGSGGGGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGPQARKGGSGGGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 24 - protein, 866 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGPQARKGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGPQARKGGSGGGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 25 - protein, 879 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGYAARKGGIGGSGGGGSGGGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGYAARKGGIGGSGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 26 - protein, 866 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGYAARKGGIGGSGGGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGYAARKGGIGGSGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 27 - protein, 415 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGPQARKGGSGGGGSGGGGSGGGGSGGGGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGPQARKGGSGGGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 28 - protein, 402 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGPQARKGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGPQARKGGSGGGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 29 - protein, 415 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGYAARKGGIGGSGGGGSGGGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGYAARKGGIGGSGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 30 - protein, 402 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGYAARKGGIGGSGGGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGYAARKGGIGGSGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 31 - protein, 214 aa, synthetic construct
DIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 447 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 252 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGSGLNDIFEAQKIEWHEARAHHHHHH

SEQ ID NO: 36 - protein, 396 aa, synthetic construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGSGLNDIFEAQKIEWHE

SEQ ID NO: 37 - protein, 162 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGLNDIFEAQKIEWHEARAHHHHHH

SEQ ID NO: 38 - protein, 447 aa, synthetic construct
EVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 41 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 447 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 594 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 44 - protein, 879 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGGGSGGGGSGGGGSGGGGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGSGGGGSGGGGSGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 45 - protein, 866 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGSGGGGSGGGGSGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 46 - protein, 597 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 47 - protein, 218 aa, synthetic construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - protein, 443 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 49 - protein, 873 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGYAARKGGIGGSGGGGSGGGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGYAARKGGIGGSGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 50 - protein, 873 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGGGSGGGGSGGGGSGGGGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGSGGGGSGGGGSGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 51 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 52 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 53 - protein, 111 aa, synthetic construct
DIVMTQSPDSLAVSLGERATINCKASESVDTSDNSFIHWYQQKPGQSPKLLIYRASTLESGVPDRFSGSGSGTDFTLTISSLQAEDVAVYYCQQNYDVPWTFGQGTKVEIK

SEQ ID NO: 54 - protein, 120 aa, synthetic construct
EVQLLESGGGLVQPGGSLRLSCAASGFSFSSYTMSWVRQAPGKGLEWVSAISGGGRDIYYPDSVKGRFTISRDNSKNTLYLQMNSLRAEDTAVYYCVLLTGRVYFALDSWGQGTLVTVSS

SEQ ID NO: 55 - protein, 107 aa, synthetic construct
DIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 56 - protein, 117 aa, synthetic construct
EVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSS

SEQ ID NO: 57 - protein, 218 aa, synthetic construct; identical to SEQ ID NO: 11

SEQ ID NO: 58 - protein, 449 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 59 - protein, 879 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGGGSGGGGSPQARKGGGGSGGGGSGGGGSGGGGSGGGEVQLVESGGGLVQPGGSLRLSCAASGFTFSSYTLAWVRQAPGKGLEWVAAIDSSSYTYSPDTVRGRFTISRDNAKNSLYLQMNSLRAEDTAVYYCARDSNWDALDYWGQGTTVTVSSGGGGSGGPQARKGGGGSGGGDIQMTQSPSSLSASVGDRVTITCKASQNVGTNVGWYQQKPGKAPKALIYSASFRYSGVPSRFSGSGSGTDFTLTISSLQPEDFATYYCQQYYTYPYTFGGGTKVEIK

SEQ ID NO: 60 - protein, 8 aa, synthetic construct
YAARKGGI

SEQ ID NO: 61 - protein, 5 aa, synthetic construct
PQARK

SEQ ID NO: 62 - protein, 133 aa, Homo sapiens [sequence omitted]

SEQ ID NO: 63 - protein, 38 aa, synthetic construct
GGSGGGGSGGPQARKGGSGGGGSGGGGSGGGGSGGGGG

SEQ ID NO: 64 - protein, 25 aa, synthetic construct
GGSGGGGSGGPQARKGGSGGGGSGG

SEQ ID NO: 65 - protein, 38 aa, synthetic construct
GGSGGGGSGGYAARKGGIGGSGGGGSGGGGSGGGGSGG

SEQ ID NO: 66 - protein, 25 aa, synthetic construct
GGSGGGGSGGYAARKGGIGGSGGGG

SEQ ID NO: 67 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 68 - protein, 15 aa, synthetic construct
KASESVDTSDNSFIH

SEQ ID NO: 69 - protein, 7 aa, synthetic construct
RASTLES

SEQ ID NO: 70 - protein, 9 aa, synthetic construct
QQNYDVPWT

SEQ ID NO: 71 - protein, 5 aa, synthetic construct
SYTMS

SEQ ID NO: 72 - protein, 17 aa, synthetic construct
AISGGGRDIYYPDSVKG

SEQ ID NO: 73 - protein, 11 aa, synthetic construct
LTGRVYFALDS

SEQ ID NO: 74 - protein, 11 aa, synthetic construct
RASQGVGTNVG

SEQ ID NO: 75 - protein, 7 aa, synthetic construct
SASFLYS

SEQ ID NO: 76 - protein, 9 aa, synthetic construct
QQYYTYPYT

SEQ ID NO: 77 - protein, 5 aa, synthetic construct
SYTLA

SEQ ID NO: 78 - protein, 16 aa, synthetic construct
AIDSSSYTYSPDTVKG

SEQ ID NO: 79 - protein, 9 aa, synthetic construct
DSNWDALDY

SEQ ID NO: 80 - protein, 215 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - protein, 689 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 450 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 218 aa, synthetic construct; identical to SEQ ID NO: 47

SEQ ID NO: 84 - protein, 860 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGPQARKGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGPQARKGGSGGGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 85 - protein, 443 aa, synthetic construct; identical to SEQ ID NO: 48

SEQ ID NO: 86 - protein, 860 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGSGGGGSGGGGSGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 87 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 88 - protein, 860 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGPQARKGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGPQARKGGSGGGGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 89 - protein, 443 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 90 - protein, 860 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGSGGGGSGGGGSGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGGSGGGGSGGGGSGGGGSGGGGSGGEVQLVESGGGLVQPGGSLRLSCAASGFTFSSYTLAWVRQAPGKGLEWVAAIDSSSYTYSPDTVKGRFTISRDNSKNTLYLQMNSLRAEDTAVYYCARDSNWDALDYWGQGTTVTVSSGGSGGGGSGGGGSGGGGSGGDIQMTQSPSSLSASVGDRVTITCRASQGVGTNVGWYQQKPGKAPKALIYSASFLYSGVPSRFSGSGSGTDFTLTISSLQPEDFATYYCQQYYTYPYTFGGGTKVEIK

SEQ ID NO: 91 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 92 - protein, 450 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 93 - protein, 591 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 94 - protein, 591 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

(19) United States
(12) Patent Application Publication
(10) Pub. No.: US 2026/0345804 A1
(43) Pub. Date: Nov. 5, 2026

(54) PROTEASE-ACTIVATED POLYPEPTIDES

(71) Applicant: Hoffmann-La Roche Inc., Little Falls, NJ (US)

(72) Inventors: Laura CODARRI DEAK, Au (CH); Greta DURINI, Zürich (CH); Linda FAHRNI, Fehraltorf (CH); Anne FREIMOSER-GRUNDSCHOBER, Zürich (CH); Ralf HOSSE, Mettmenstetten (CH); Christian KLEIN, Bonstetten (CH); Ekkehard MOESSNER, Kreuzlingen (CH); Valeria NICOLINI, Erlenbach (CH); Pablo UMAÑA, Lachen (CH); Inja WALDHAUER, Zürich (CH)

(73) Assignee: Hoffmann-La Roche Inc., Little Falls, NJ (US)

(21) Appl. No.: 19/116,977

(22) PCT Filed: Sep. 27, 2023
(86) PCT No.: PCT/EP2023/076655

§ 371 Date: Mar. 28, 2025

(30) Foreign Application Priority Data
Sep. 29, 2022 (EP) 22198666.4

(51) Int. Cl. A61K 47/68; A61P 35/00; C07K 14/55; C07K 16/24

(52) U.S. Cl. CPC A61K 47/6813; A61K 47/6851; A61P 35/00; C07K 14/55; C07K 16/246

(57) ABSTRACT

The present invention generally relates to novel protease-activatable interleukin-2 (IL-2) polypeptides and immunoconjugates comprising (i) an IL-2 polypeptide, (ii) a masking moiety and (iii) a linker comprising a first protease cleavage site, wherein the linker has a length of 20 to 45 amino acids, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site. The present invention also relates to polynucleotides encoding such protease-activatable interleukin-2 (IL-2) polypeptides and immunoconjugates, and vectors and host cells comprising such polynucleotides. The invention further relates to methods for producing the protease-activatable interleukin-2 (IL-2) polypeptides and immunoconjugates of the invention, and to methods of using these protease-activatable interleukin-2 (IL-2) polypeptides and immuno-conjugates in the treatment of disease.

### FIELD OF THE INVENTION

The present invention generally relates to novel protease-activated polypeptides, particularly interleukin-2 (IL-2) polypeptides. More particularly, the invention concerns protease-activated IL-2 polypeptides that exhibit improved properties for use as immunotherapeutic agents. In addition, the invention relates to protease-activated IL-2 polypeptides or immunoconjugates, polynucleotides, vectors and host cells comprising such vectors or polynucleotide molecules. The invention further relates to methods for producing the protease-activated IL-2 polypeptides or immunoconjugates, pharmaceutical compositions comprising the same, and uses thereof.

### BACKGROUND

The selective destruction of an individual target cell or a specific target cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. In this regard, conjugates designed to bind to a surface antigen on target cells and comprising interleukin-2 (IL-2) variants are supposed to activate nearby T effector cells and NK cells. The simultaneous binding of such an conjugate to its target and the interleukin-2 receptor will cause activation of T effector cells and NK cells in the proximity of the target (in trans) or when the target is expressed on the T effector cell and NK cell, this cell is activated upon binding (in cis).

Interleukin-2 (IL-2), also known as T cell growth factor (TCGF), is a 15.5 kDa globular glycoprotein playing a central role in lymphocyte generation, survival and homeostasis. It has a length of 133 amino acids and consists of four antiparallel, amphiphatic α-helices that form a quaternary structure indispensable of its function (Smith, Science 240, 1169-76 (1988); Bazan, Science 257, 410-413 (1992)). Sequences of IL-2 from different species are found under NCBI RefSeq Nos. NP000577 (human), NP032392 (mouse), NP446288 (rat) or NP517425 (chimpanzee).

IL-2 mediates its action by binding to IL-2 receptors (IL-2R), which consist of up to three individual subunits, the different association of which can produce receptor forms that differ in their affinity to IL-2. Association of the α (CD25), β (CD122), and γ (γ_{c}, CD132) subunits results in a trimeric, high-affinity receptor for IL-2. Dimeric IL-2 receptor consisting of the β and γ subunits is termed intermediate-affinity IL-2R. The a subunit forms the monomeric low affinity IL-2 receptor. Although the dimeric intermediate-affinity IL-2 receptor binds IL-2 with approximately 100-fold lower affinity than the trimeric high-affinity receptor, both the dimeric and the trimeric IL-2 receptor variants are able to transmit signal upon IL-2 binding (Minami et al., Annu Rev Immunol 11, 245-268 (1993)). Hence, the α-subunit, CD25, is not essential for IL-2 signalling. It confers high-affinity binding to its receptor, whereas the β subunit, CD122, and the γ-subunit are crucial for signal transduction (Krieg et al., Proc Natl Acad Sci 107, 11906-11 (2010)). Trimeric IL-2 receptors including CD25 are expressed by (resting) CD4⁺ forkhead box P3 (FoxP3)⁺ regulatory T (T_{reg}) cells. They are also transiently induced on conventional activated T cells, whereas in the resting state these cells express only dimeric IL-2 receptors. T_{reg} cells consistently express the highest level of CD25 in vivo (Fontenot et al., Nature Immunol 6, 1142-51 (2005)).

IL-2 is synthesized mainly by activated T-cells, in particular CD4⁺ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (reviewed e.g. in Waldmann, Nat Rev Immunol 6, 595-601 (2009); Olejniczak and Kasprzak, Med Sci Monit 14, RA179-89 (2008); Malek, Annu Rev Immunol 26, 453-79 (2008)).

Its ability to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma.

However, IL-2 has a dual function in the immune response in that it not only mediates expansion and activity of effector cells, but also is crucially involved in maintaining peripheral immune tolerance.

A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD3 complex, the expression of Fas ligand (FasL) and/or tumor necrosis factor (TNF) is induced, making the cells susceptible for Fas-mediated apoptosis. This process is IL-2 dependent (Lenardo, Nature 353, 858-61 (1991)) and mediated via STAT5. By the process of AICD in T lymphocytes tolerance cannot only be established to self-antigens, but also to persistent antigens that are clearly not part of the host's makeup, such as tumor antigens.

Moreover, IL-2 is also involved in the maintenance of peripheral CD4⁺ CD25⁺ regulatory T (T_{reg}) cells (Fontenot et al., Nature Immunol 6, 1142-51 (2005); D'Cruz and Klein, Nature Immunol 6, 1152-59 (2005); Maloy and Powrie, Nature Immunol 6, 1171-72 (2005), which are also known as suppressor T cells. They suppress effector T cells from destroying their (self-) target, either through cell-cell contact by inhibiting T cell help and activation, or through release of immunosuppressive cytokines such as IL-10 or TGF-β. Depletion of T_{reg} cells was shown to enhance IL-2 induced anti-tumor immunity (Imai et al., Cancer Sci 98, 416-23 (2007)).

Therefore, IL-2 is not optimal for inhibiting tumor growth, because in the presence of IL-2 either the CTLs generated might recognize the tumor as self and undergo AICD or the immune response might be inhibited by IL-2 dependent T_{reg} cells.

A further concern in relation to IL-2 immunotherapy are the side effects produced by recombinant human IL-2 treatment. Patients receiving high-dose IL-2 treatment frequently experience severe cardiovascular, pulmonary, renal, hepatic, gastrointestinal, neurological, cutaneous, haematological and systemic adverse events, which require intensive monitoring and in-patient management. The majority of these side effects can be explained by the development of so-called vascular (or capillary) leak syndrome (VLS), a pathological increase in vascular permeability leading to fluid extravasation in multiple organs (causing e.g. pulmonary and cutaneous edema and liver cell damage) and intravascular fluid depletion (causing a drop in blood pressure and compensatory increase in heart rate). There is no treatment of VLS other than withdrawal of IL-2. Low-dose IL-2 regimens have been tested in patients to avoid VLS, however, at the expense of suboptimal therapeutic results. VLS was believed to be caused by the release of proinflammatory cytokines, such as tumor necrosis factor (TNF)-α from IL-2-activated NK cells, however it has recently been shown that IL-2-induced pulmonary edema resulted from direct binding of IL-2 to lung endothelial cells, which expressed low to intermediate levels of functional αβγ IL-2 receptors (Krieg et al., Proc Nat Acad Sci USA 107, 11906-11 (2010)).

Several approaches have been taken to overcome these problems associated with IL-2 immunotherapy. For example, it has been found that the combination of IL-2 with certain anti-IL-2 monoclonal antibodies enhances treatment effects of IL-2 in vivo (Kamimura et al., J Immunol 177, 306-14 (2006); Boyman et al., Science 311, 1924-27 (2006)). In an alternative approach, IL-2 has been mutated in various ways to reduce its toxicity and/or increase its efficacy. Hu et al. (Blood 101, 4853-4861 (2003), US Pat. Publ. No. 2003/0124678) have substituted the arginine residue in position 38 of IL-2 by tryptophan to eliminate IL-2's vasopermeability activity. Shanafelt et al. (Nature Biotechnol 18, 1197-1202 (2000)) have mutated asparagine 88 to arginine to enhance selectivity for T cells over NK cells. Heaton et al. (Cancer Res 53, 2597-602 (1993); U.S. Pat. No. 5,229,109) have introduced two mutations, Arg38Ala and Phe42Lys, to reduce the secretion of proinflammatory cytokines from NK cells. Gillies et al. (US Pat. Publ. No. 2007/0036752) have substituted three residues of IL-2 (Asp20Thr, Asn88Arg, and Gln126Asp) that contribute to affinity for the intermediate-affinity IL-2 receptor to reduce VLS. Gillies et al. (WO 2008/0034473) have also mutated the interface of IL-2 with CD25 by amino acid substitution Arg38Trp and Phe42Lys to reduce interaction with CD25 and activation of T_{reg} cells for enhancing efficacy. To the same aim, Wittrup et al. (WO 2009/061853) have produced IL-2 mutants that have enhanced affinity to CD25, but do not activate the receptor, thus act as antagonists. The mutations introduced were aimed at disrupting the interaction with the β- and/or γ-subunit of the receptor.

A particular mutant IL-2 polypeptide, designed to overcome the above-mentioned problems associated with IL-2 immunotherapy (toxicity caused by the induction of VLS, tumor tolerance caused by the induction of AICD, and immunosuppression caused by activation of Treg cells), is described in WO 2012/107417. Substitution of the phenylalanine residue at position 42 by alanine, the tyrosine residue at position 45 by alanine and the leucine residue at position 72 of IL-2 by glycine essentially abolishes binding of this mutant IL-2 polypeptide to the α-subunit of the IL-2 receptor (CD25).

However, none of the known IL-2 mutants was shown to overcome all of the above-mentioned problems associated with IL-2 immunotherapy, namely toxicity caused by the induction of VLS, tumor tolerance caused by the induction of AICD, and immunosuppression caused by activation of T_{reg} cells.

Further to the above-mentioned approaches, IL-2 immunotherapy may be improved by selectively targeting IL-2 to tumors, e.g. in the form of immunoconjugates comprising an antibody that binds to an antigen expressed on tumor cells or that binds to effector cells in the tumor environment. Several such immunoconjugates have been described (see e.g. Ko et al., J Immunother (2004) 27, 232-239; Klein et al., Oncoimmunology (2017) 6(3), e1277306; WO 2018/184964 A1).

Given the clinical success and unprecedented efficacy of PD-1/PD-L1 check-point inhibitors there remains a major medical need to increase the response rate and duration in patients with pre-existing T cell immunity further. Recent reports have demonstrated that two populations of tumor-specific CD8 T cells are targeted by PD-1 antibodies: the exhausted TILs and, their newly described TCF1+ precursor with stem-like properties, TResource cells. Of the two the latter correlates with a favorable disease prognosis and response to anti-PD-1 therapy. Cytokines, like interleukin-2, have also been described to induce the proliferation/differentiation of the TResource cells towards functional effector T cells.

IL-2 has been the first effective cancer immunotherapy used to treat metastatic melanoma and renal cell carcinoma. Unfortunately, IL-2, at high concentrations, is toxic by inducing vascular leak syndrome (VLS) and detrimentally expands regulatory T cells and induces activation induced cell death due to binding to CD25. In order to overcome these limitations of wildtype IL2/Proleukin, IL-2v variants with abolished CD25 binding have been described. However, due to the mechanism of IL-2 signaling through the heterodimeric intermediate affinity IL-2Rbg complex IL2v and other IL2 variants automatically activate IL-2R signaling as soon as they encounter the IL-2R and as a consequence mediate unspecific and peripheral immune cell activation outside of the tumor in blood, the vasculature and lymphoid tissues resulting in dose limiting toxicities. As a consequence, it is not possible to administer to a patients as much IL-2 or IL2v as desired in order to achieve the maximal therapeutic benefit.

Taken together, by targeting PD1-IL2v in cis to PD-1⁺ T cells a stronger therapeutic effect of PD1-IL2v can be achieved. In fact, cis-targeting of PD1-IL2v to appropriate antigen specific T-cell subsets, together with PD-1/-L1 inhibition is a better way to exploit endogenous immunity therapeutically and one of the strongest immunomodulatory pathway known for unleashing endogenous immunity for cancer immunotherapy. However, due to the possibility of the IL2v moiety to trigger IL-2R signaling in the periphery also for PD1-IL2v not the maximally desired dose can be administered due to the peripheral non-tumor specific IL-2R activation. Thus, the therapeutic index is believed to remain narrow with an anticipated MTD with a flat dose of >10-30 mg in man which may limit utilizing full pathway potential. CD8 T cells could be targeted instead, as well as other T cell targets.

Therefore, it is critical to generate next generations of IL-2 molecules cis-targeted to antigen-experienced T cells but with wider therapeutic index.

Serine proteases (e.g. matriptase), cysteine proteases (e.g. cathepsin S) and matrix metalloproteinases (e.g. MMP-2 and MMP-9) are overexpressed in several cancer types (Duffy, M. J. Proteases as prognostic markers in cancer. Clin. Cancer Res. 2, 613-618 (1996)). Matriptase, matrix metalloproteinase 2 (MMP-2, gelatinase A) and matrix metalloproteinase 9 (MMP-9, gelatinase B) are overexpressed e.g. in breast- and ovarian carcinoma (McGowan, P. M. & Duffy, M. J. Matrix metalloproteinase expression and outcome in patients with breast cancer: analysis of a published database. Ann. Oncol. 19, 1566-1572 (2008)). MMP-2 and MMP-9 activity was detected in cervical, breast and ovarian carcinoma and ascites of patients with epithelial ovarian cancer (EOC) but not in the serum of these patients (Demeter, A. et al. Molecular prognostic markers in recurrent and in non-recurrent epithelial ovarian cancer. Anticancer Res. 25, 2885-2889 (2005)). While matriptase can be detected in normal epithelial cells, matriptase activity is mainly detected in cancer (LeBeau, A. M. et al. Imaging a functional tumorigenic biomarker in the transformed epithelium. Proc. Natl. Acad. Sci. USA 110, 93-98 (2013)).

Although current immunotherapies directed against the PD1/PDL1 axis have shown unprecedented efficacy in several cancer indications, there is a substantial portion of patients who do not respond to the treatment or relapse, while other tumor-types remain largely refractory to these therapies. Therefore, there is a clear, high unmet need of a considerable cancer patient population in patients that have some kind of pre-existing T cell immune response. Examples for indications where PD1 antagonism has resulted in objective responses are e.g. advanced or metastatic melanoma, Merkel cell carcinoma, NSCLC, SCLC, RCC, gastric cancer, hepatocellular cancer, head and neck carcinoma, breast cancer, ovarian cancer, mismatch repair deficient versus sufficient CRC and haematological malignancies like DLBCL and PMBCL after autologous stem cell transplant and HL (Editiorial: PD-Loma: a cancer entity with a shared sensitivity to the PD-1/PD-L1 pathway blockade, British Journal of Cancer (2019) 120:3-5; https://doi.org/10.1038/s41416-018-0294-4).

The task of generating IL-2 variants and conjugates suitable for treatment provides several technical challenges related to efficacy, toxicity, applicability and produceability that have to be met. In instances where the conjugate targets an antigen on a target cell, e.g., a cancer cell, that is also expressed in non-target tissue, toxicity can occur. Thus there remains a need in the art to further enhance the therapeutic usefulness of IL-2 polypeptides.

### SUMMARY OF THE INVENTION

The present invention is based, in part, on the recognition that tumor environments (TME) highly express proteases compared to normal tissue and that a masked therapeutic agent, preferably protease-activatable interleukin-2, has a reduced or abolished systemic activity and full activity in the tumor environment upon activation by proteases.

Accordingly, a first aspect of the invention provides a protease-activatable interleukin-2 (IL-2) polypeptide comprising (i) an IL-2 polypeptide, (ii) a masking moiety and (iii) a linker comprising a first protease cleavage site, wherein the linker has a length of 20 to 45 amino acids, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site. In one embodiment, the linker has a length of 22 to 43 amino acids. In one embodiment, the linker has a length of 25 to 38 amino acids. In one embodiment, the linker has a length of 25 amino acids, preferably the linker has the sequence according to SEQ ID NO: 64 or SEQ ID NO: 66. In one embodiment, the linker has a length of 38 amino acids, preferably the linker has a sequence according to SEQ ID NO: 63 or SEQ ID NO: 65. In one embodiment, the masking moiety is covalently attached to the amino-terminus or carboxy-terminus of the interleukin-2 polypeptide through the linker. In one embodiment, the masking moiety is an IL-2 antagonist. In one embodiment, the masking moiety is an IL-2 antibody or an IL-2 receptor subunit. In one embodiment, the IL-2 antibody comprises a Fab molecule. In one embodiment, the masking moiety is an antibody derived from MT204, preferably MT204. The MT204 antibody is disclosed e.g. in Volkland et al., Molecular Immunology 44 (2007) 1743-1753, and PCT-publication WO 2006/128690 A1. More preferably, the masking moiety is a de-immunized MT204-derived binder. In one embodiment, the Fab molecule is a single-chain Fab molecule. In one embodiment, the second protease cleavage site is located between the variable domain of the heavy chain (VH) and the variable domain of the light chain (VL) of the single-chain Fab molecule. In one embodiment, the first protease cleavage site and the second protease cleavage site each comprise at least one protease recognition sequence. In one embodiment, the protease recognition sequence of the first protease cleavage site and/or the protease recognition sequence of the second protease cleavage site is either YAARKGGI according to SEQ ID NO:60 and/or PQARK according to SEQ ID NO:61.

In one embodiment, the IL-2 polypeptide is a wild-type IL-2, preferably a human IL-2 according to SEQ ID NO: 62, or a mutant IL-2 polypeptide. In one embodiment, the mutant IL-2 polypeptide comprises any amino acid substitution selected from the group T3A, F42A, Y45A, L72G, C125A of human IL-2 according to SEQ ID NO:62. In one embodiment, the mutant IL-2 polypeptide comprises the amino acid substitutions F42A, Y45A and L72G of human IL-2 according to SEQ ID NO:62. In one embodiment, the mutant IL-2 polypeptide comprises the amino acid substitutions T3A, F42A, Y45A, L72G and C125A of human IL-2 according to SEQ ID NO: 62. In one embodiment, the protease-activatable IL-2 polypeptide comprises the amino acid sequence of SEQ ID NO: 27, SEQ ID NO: 28, SEQ ID NO: 29 or SEQ ID NO: 30. In one embodiment, the IL-2 polypeptide is further attached to a non-IL-2 moiety. In one embodiment, the IL-2 polypeptide shares a carboxy-terminal peptide bond with the masking moiety and an amino-terminal peptide bond with the non-IL-2 moiety or the IL-2 polypeptide shares an amino-terminal peptide bond with the masking moiety and a carboxy-terminal peptide bond with the non-IL-2 moiety. In one embodiment the non-IL-2 moiety is an antigen binding moiety or an effector cell binding moiety.

In a further aspect, the invention provides an immunoconjugate comprising a protease-activatable IL-2 polypeptide as described herein and an antigen binding moiety and/or an effector cell binding moiety. In one embodiment, the protease-activatable IL-2 polypeptide shares an amino- or carboxy-terminal peptide bond with the antigen binding moiety or the effector cell binding moiety. In one embodiment, the immunoconjugate comprises a first and a second antigen binding moiety or a first and a second effector cell antigen binding moiety or an antigen binding moiety and an effector cell binding moiety. In one embodiment, (i) the protease-activatable IL-2 polypeptide shares an amino- or carboxy-terminal peptide bond with said first antigen binding moiety and said second antigen binding moiety shares an amino- or carboxy-terminal peptide bond with either a) said protease-activatable IL-2 polypeptide or b) said first antigen binding moiety; (ii) the protease-activatable IL-2 polypeptide shares an amino- or carboxy-terminal peptide bond with said first effector cell binding moiety and said second effector cell binding moiety shares an amino- or carboxy-terminal peptide bond with either a) said protease-activatable IL-2 polypeptide or b) said first effector cell binding moiety; (iii) the protease-activatable IL-2 polypeptide shares an amino- or carboxy-terminal peptide bond with the antigen binding moiety and the effector cell binding moiety shares an amino- or carboxy-terminal peptide bond with either a) said protease-activatable IL-2 polypeptide or b) said antigen binding moiety; or (iv) the protease-activatable IL-2 polypeptide shares an amino- or carboxy-terminal peptide bond with the effector cell binding moiety and the antigen binding moiety shares an amino- or carboxy-terminal peptide bond with either a) said protease-activatable IL-2 polypeptide or b) said effector cell binding moiety.

In one embodiment, the antigen binding moiety or the effector cell binding moiety comprised in the protease-activatable IL-2 polypeptide as disclosed herein or the immunoconjugate as disclosed herein is an antibody or an antibody fragment. In one embodiment, the antigen binding moiety and/or said effector cell binding moiety is a Fab molecule or scFv molecule. In one embodiment, the antigen binding moiety and/or said effector cell binding moiety is an immunoglobulin molecule, particularly an IgG molecule. In one embodiment, the antigen binding moiety is directed to an antigen presented on a tumor cell or in a tumor cell environment and/or wherein said effector cell binding moiety is directed to an effector cell present in a tumor cell environment in order to achieve cis-targeting.

In one embodiment, (i) the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 23; (ii) the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 24; (iii) the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 25; or (iv) the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 26.

In one embodiment, (i) the immunoconjugate comprises an amino acid sequence according to SEQ ID NO: 5, an amino acid sequence according to SEQ ID NO: 22, and an amino acid sequence according to SEQ ID NO: 23; (ii) the immunoconjugate comprises an amino acid sequence according to SEQ ID NO: 5, an amino acid sequence according to SEQ ID NO: 22, and an amino acid sequence according to SEQ ID NO: 24; (iii) the immunoconjugate comprises an amino acid sequence according to SEQ ID NO: 5, an amino acid sequence according to SEQ ID NO: 22, and an amino acid sequence according to SEQ ID NO: 25; or (iv) the immunoconjugate comprises an amino acid sequence according to SEQ ID NO: 5, an amino acid sequence according to SEQ ID NO: 22, and an amino acid sequence according to SEQ ID NO: 26.

The invention further provides one or more isolated polynucleotide encoding the protease-activatable IL-2 polypeptide as described herein or the immunoconjugate as described herein, one or more expression vectors comprising the polynucleotide as described herein, one or more host cells comprising the polynucleotide as described herein or the expression vector as described herein.

Also provided is a method of producing a protease-activatable IL-2 polypeptide or an immunoconjugate as described herein comprising culturing the host cell as described herein under conditions suitable for the expression of the protease-activatable IL-2 polypeptide or the immunoconjugate.

Also provided is a protease-activatable IL-2 polypeptide or immunoconjugate as described herein produced by the method described herein. Also provided is a pharmaceutical composition comprising the protease-activatable IL-2 polypeptide or immunoconjugate as disclosed herein and a pharmaceutically acceptable carrier. In particular, the invention encompasses a proteaseactivatable IL-2 polypeptide or an immunoconjugate as described herein for use in the treatment of a disease in an individual in need thereof. In a particular embodiment, said disease is cancer. In a particular embodiment, the individual is a human.

Also encompassed by the invention is the use of the protease-activatable IL-2 polypeptide or immunoconjugate as described herein for manufacture of a medicament for treating a disease in an individual in need thereof. Further provided is a method of treating a disease in an individual, comprising administering to said individual a therapeutically effective amount of a composition comprising the protease-activatable IL-2 polypeptide or immunoconjugate as described herein in a pharmaceutically acceptable form. Said disease preferably is cancer.

Also provided is a method of stimulating the immune system of an individual, comprising administering to said individual an effective amount of a composition comprising the proteaseactivatable IL-2 polypeptide or immunoconjugate as disclosed herein in a pharmaceutically acceptable form.

### BRIEF DESCRIPTION OF THE FIGURES

FIG. 1A-I. IgG antibodies generated to assess the de-immunized MT204 masks and anti-PD1 binder variants by SPR and respective antigens and parental controls. FIG. 1A (P1AH2050-P1AH2052) De-immunized MT204 masks in human IgG PG LALA format; FIG. 1B (P1AH4157-P1AH4161) First set of de-immunized anti-PD1 binder variants in human IgG PG LALA format; FIG. 1C (P1AI0356-P1AI0360) Second set of de-immunized anti-PD1 binder variants in human IgG PG LALA format; FIG. 1D (P1AI1648-P1AI1652) Third set of de-immunized anti-PD1 binder variants in human IgG PG LALA format; FIG. 1E (P1AF7506) One-armed parental MT204 in human IgG PG LALA format; FIG. 1F (P1AA6888) Parental anti-PD1 binder in human IgG PG LALA format; FIG. 1G (P1AD9704) Human PD1 antigen as Fc-fusion with C-terminal biotinylated avi-tags and His-tag; FIG. 1H (P1AG0879) IL2v cytokine with C-terminal avi-tag and His-tag; FIG. 1I (P1AA9690) Parental MT204 in human IgG PG LALA format.

FIG. 2A-F. Human PD1-targeted masked IL2v constructs with PQARK or YAARKGGI matriptase sites and respective non-masked PD1-targeted or FAP-targeted controls. FIG. 2A (P1AI4322) Bivalent human PD1-targeted human IgG PG LALA with masked IL2v (‘in-line’) fused to the C-terminus of the Fc knob chain, 2 PQARK matriptase sites for the release of the mask and a 38 amino acid linker between the IL2v cytokine and the scFv mask; FIG. 2B (P1AI4323) Bivalent human PD1-targeted human IgG PG LALA with masked IL2v (‘in-line’) fused to the C-terminus of the Fc knob chain, 2 PQARK matriptase sites for the release of the mask and a 25 amino acid linker between the IL2v cytokine and the scFv mask; FIG. 2C (P1AI4324) Bivalent human PD1-targeted human IgG PG LALA with masked IL2v (‘in-line’) fused to the C-terminus of the Fc knob chain, 2 YAARKGGI matriptase sites for the release of the mask and a 38 amino acid linker between the IL2v cytokine and the scFv mask; FIG. 2D (P1AI4325) Bivalent human PD1-targeted human IgG PG LALA with masked IL2v (‘in-line’) fused to the C-terminus of the Fc knob chain, 2 YAARKGGI matriptase sites for the release of the mask and a 25 amino acid linker between the IL2v cytokine and the scFv mask; Constructs A-E comprise the preferred de-immunized V-domain sequences of the anti-PD1 binder and the MT204 scFv mask; FIG. 2E (P1AE4422) Bivalent human PD1-targeted human IgG PG LALA with IL2v fused to the C-terminus of the Fc knob chain as non-masked control; FIG. 2F (P1AA5355) Bivalent human FAP-targeted human IgG PG LALA with IL2v fused to the C-terminus of the Fc knob chain as non-masked control.

FIG. 3A-C. Human PD1-targeted masked IL2v constructs as non-cleavable controls and respective non-masked control. FIG. 3A (P1AI4646) Bivalent human PD1-targeted human IgG PG LALA with masked IL2v (‘in-line’) fused to the C-terminus of the Fc knob chain, a 38 amino acid linker between the IL2v cytokine and the scFv mask, and without matriptase release sites (non-cleavable control); FIG. 3B (P1AI4647) Bivalent human PD1-targeted human IgG PG LALA with masked IL2v (‘in-line’) fused to the C-terminus of the Fc knob chain, a 25 amino acid linker between the IL2v cytokine and the scFv mask, and without matriptase release sites (non-cleavable control); FIG. 3C (P1AI4648) Bivalent human PD1-targeted human IgG PG LALA with IL2v fused to the C-terminus of the Fc knob chain as non-masked control; Constructs A-C comprise the preferred de-immunized V-domain sequences of the anti-PD1 binder and the MT204 scFv mask (constructs A and B).

FIG. 4A-B. Murine surrogates of human PD1-targeted masked IL2v constructs with YAARKGGI matriptase sites and respective control. FIG. 4A (P1AI4650) Bivalent human PD1-targeted murine IgG DA PG with masked IL2v (‘in-line’) fused to the C-terminus of the Fc DD-chain and 2 YAARKGGI matriptase sites for the release of the mask; FIG. 4B (P1AI4651) Bivalent human PD1-targeted murine IgG DA PG with masked IL2v (‘in-line’) fused to the C-terminus of the Fc DD-chain and without matriptase release sites (non-cleavable control).

FIG. 5A-B. Measurement of the potency of de-immunized aPD-1 constructs to block the PD1/PD-L1 interaction. PD-1 Effector cells were incubated with PD-L1 aAPC/CHO-K1 in the presence of anti-PD1 antibodies. Bio-Glo Reagent was added and luminescence measured at the luminometer. Data were analyzed using GraphPad Prism software (mean±SEM). Data of 1 experiment. FIG. 5A shows blockade with P1AH4159, P1AH4160 and P1AH4161. FIG. 5B shows blockade with P1AH4157 and P1AH4158.

FIG. 6A-B. Binding of de-immunized aPD-1 to CD3/CD28 activated CD4 T cells, in comparison to aPD-1 IgG PG LALA, PD1-IL2v and FAP-IL2v. PD1 antibody constructs were added to activated CD4 T cells at different concentrations and the dose-dependent response was measured by flow cytometry. Data were analyzed using GraphPad Prism software (mean±SEM). Data from 2 donors. FIG. 6A shows binding with P1AH4159, P1AH4160 and P1AH4161. FIG. 6B shows binding with P1AH4157 and P1AH4158.

FIG. 7A-B. Binding of de-immunized aPD-1 to CD3/CD28 activated CD4 T cells, in comparison to aPD-1 IgG PG LALA, PD1-IL2v and FAP-IL2v. PD1 antibody constructs were added to activated CD4 T cells at different concentrations and the dose-dependent response was measured by flow cytometry. Data were analyzed using GraphPad Prism software (mean±SEM). Data from 2 donors. FIG. 7A shows binding with P1AH4159, P1AH4160 and P1AH4161. FIG. 7B shows binding with P1AH4157 and P1AH4158.

FIG. 8A-B. Minimal Mixed Lymphocytes Reaction. The effect of several anti-PD-1 blocking antibodies on the increase of allospecific T cell effector function was investigated by depicting the frequencies of Granzyme B production in proliferating CD4 T cells. Data were acquired by flow cytometry and analyzed using GraphPad Prism software (mean±SEM). Data from 1 experiment (CD4 T cells from 2 donors and mDCs from 2 donors). FIG. 8A shows Granzyme B production upon exposure to P1AH4159, P1AH4160 and P1AH4161. FIG. 8B shows Granzyme B production upon exposure to P1AH4157 and P1AH4158.

FIG. 9A-B. Minimal Mixed Lymphocytes Reaction. The effect of several anti-PD-1 blocking antibodies on the increase of allospecific T cell effector function was investigated by depicting the frequencies of Interferon-γ production in proliferating CD4 T cells. Data were acquired by flow cytometry and analyzed using GraphPad Prism software (mean±SEM). Data from 1 experiment (CD4 T cells from 2 donors and mDCs from 2 donors). FIG. 9A shows Interferon-γ production upon exposure to P1AH4159, P1AH4160 and P1AH4161. FIG. 9B shows Interferon-γ production upon exposure to P1AH4157 and P1AH4158.

FIG. 10. Binding of de-immunized PD1-IgGs to human PD1 overexpressing CHO cells was determined by flow cytometry. Molecules were detected with a fluorescently labeled anti-human Fc specific secondary antibody.

FIG. 11. Blocking of IL2v activity with de-immunized MT204 masks compared to the parental mask was determined by measuring proliferation of human NK92 cells using CellTiter Glo.

FIG. 12A-B. Binding of the indicated TA PD1-IL2v constructs to human PD1 overexpressing CHO cells was determined by flow cytometry. Molecules were detected with a fluorescently labeled anti-human Fc specific secondary antibody. FIG. 12A relates to constructs with the PQARK cleavage sites. FIG. 12B relates to constructs with the YAARKGGI cleavage sites.

FIG. 13A-F. Proliferation of KHYG-1 cells induced by a set of TA PD1-IL2v constructs was measured using CellTiter Glo. FIG. 13A and FIG. 13B relate to constructs with the PQARK cleavage site. FIG. 13C and FIG. 13D relate to constructs with the YARKGGI cleavage site. FIG. 13E and FIG. 13F relate to constructs without cleavage site.

FIG. 14. Results of an efficacy experiment with TA-PD1-IL2v cleavable (YAARKGGI 38 mer linker) and non-cleavable Mabs as single agents are presented. The MCA205 fibrosarcoma carcinoma cell line was injected subcutaneously in Black 6-huPD1 tg mice to study tumor growth inhibition in a subcutaneous model. Tumor size was measured using a caliper. Therapy started when tumors reached 150 mm³. The amount of antibodies injected per mouse was 2 mg/kg for TA-PD1-IL2v YAARKGGI 38 mer cleavable and TA-PD1-IL2v non-cleavable given twice a week. The treatment lasted 1 week. The TA-PD-IL2v YAARKGGI 38 mer mediated superior efficacy in terms of tumor growth inhibition compared to vehicle and non-cleavable Mab single agent groups

FIG. 15. Combination partner (human FolR1-targeted T cell engager) for in vivo efficacy studies. (P1AK1120) 2+1 human FolR1-targeted T cell engager used as combination partner in in vivo efficacy studies.

FIG. 16A-C. Murine surrogates of human PD1-targeted masked IL2v constructs with and without PQARK matriptase sites. FIG. 16A: (P1AK3638) Bivalent human PD1-targeted murine IgG DA PG with masked IL2v (‘in-line’) fused to the C-terminus of the Fc DD-chain and 2 PQARK matriptase sites for the release of the mask; FIG. 16B: (P1AK3649) Bivalent human PD1-targeted murine IgG DA PG with masked IL2v (‘in-line’) fused to the C-terminus of the Fc DD-chain and without matriptase release sites (non-cleavable control). FIG. 16C: (P1AG7552) Bivalent human PD1-targeted murine IgG DA PG with IL2v fused to the C-terminus of the Fc DD-chain (non-masked control).

FIG. 17A-D. Murine surrogates of murine PD1-targeted masked IL2v constructs with and without PQARK matriptase sites and control IgG. FIG. 17A: (P1AK3641) Bivalent murine PD1-targeted murine IgG DA PG with masked IL2v (‘in-line’) fused to the C-terminus of the Fc DD-chain and 2 PQARK matriptase sites for the release of the mask; FIG. 17B: (P1AK3640) Bivalent murine PD1-targeted murine IgG DA PG with masked IL2v (‘in-line’) fused to the C-terminus of the Fc DD-chain and without matriptase release sites (non-cleavable control); FIG. 17C: (P1AD4006) Murine PD1-targeted murine IgG used as non-IL2v-fused control construct. FIG. 17D: (P1AG9991) Bivalent murine PD1-targeted murine IgG DA PG with IL2v fused to the C-terminus of the Fc DD-chain (non-masked control).

FIG. 18A-B. HEK blue IL2 reporter cell assay with HEK blue IL2 cells overexpressing human PD1 to test activity of murinized TA PD1-IL2v constructs. Constructs were predigested with recombinant human Matripatase (FIG. 18A). Constructs were tested without predigestion (FIG. 18B).

FIG. 19A-B. HEK blue IL2 reporter cell assay with HEK blue IL2 cells overexpressing mouse PD1 to test activity of murinized TA PD1-IL2v constructs containing a mouse specific PD1 binder. Constructs were predigested with recombinant human Matriptase (FIG. 19A). Constructs were tested without predigestion (FIG. 19B).

FIG. 20A-B. Proliferation of NK cells (FIG. 20A) and CD8 T cells (FIG. 20B) measured by CFSE dilution upon treatment with TA PD1-IL2v constructs that were digested with recombinant human Matriptase.

FIG. 21A-B. Activation of NK cells (FIG. 21A) and CD8 T cells (FIG. 21B) measured by CD25 upregulation upon treatment with TA PD1-IL2v constructs that were digested with recombinant human Matriptase.

FIG. 22. Presents the results of an efficacy experiment with TA-PD1-IL2v cleavable (PQARK 25 mer linker), non-cleavable and Pembrolizumab Mabs as single agents. The MCA205 fibrosarcoma carcinoma cell line was injected subcutaneously in Black 6-huPD1 tg mice to study tumor growth inhibition in a subcutaneous model. Tumor size was measured using a caliper. Therapy started when tumors reached 200 mm³. The amount of antibodies injected per mouse was 1 and 3 mg/kg for TA-PD1-IL2v PQARK 25 mer cleavable and 3 mg/kg for Pembrolizumab and TA-PD1-IL2v non-cleavable given twice a week. The treatment lasted 1 week. The TA-PD-IL2v PQARK 25 mer mediated superior efficacy in terms of tumor growth inhibition compared to vehicle, Pembrolizumab and non-cleavable Mabs single agent groups.

FIG. 23. Presents the results of an efficacy experiment with muTA-PD1-IL2v cleavable (PQARK 25 mer linker) and muPD1 Mabs as single agents. The GL261 glioblastoma cell line was injected subcutaneously in Black 6 mice to study tumor growth inhibition in a subcutaneous model. Tumor size was measured using a caliper. Therapy started when tumors reached 100 mm³. The amount of antibodies injected per mouse was 1 and 3 mg/kg for muTA-PD1-IL2v PQARK 25 mer cleavable and 3 mg/kg for muPD1 given twice a week. The treatment lasted 1 week. The mu-TA-PD-IL2v PQARK 25 mer mediated superior efficacy in terms of tumor growth inhibition compared to vehicle and muPD1 Mabs single agent groups.

FIG. 24. Presents the results of an efficacy experiment evaluating TA-PD1-IL2v and FOLR1-TCB Mabs in combination. The BC004 human breast carcinoma PDX cells were injected subcutaneously in humanized NSG mice to study tumor growth inhibition in a breast subcutaneous xenograft model. The amount of antibodies injected per mouse in mg/kg is the following: 1 mg/kg TA-PD1-IL2v PQARK 25 mer cleavable linker, 0.1 mg/kg PD1-IL2v non masked, 1 mg/kg Pembrolizumab and 0.3 mg/kg FOLR1-TCB Mabs. The antibodies were injected iv once weekly for 4 weeks. Significant superior tumor growth inhibition was observed in the combination FOLR1-TCB+TA-PD1-IL2v PQARK 25 mer cleavable linker group compared to FOLR1-TCB single agent and FOLR1-TCB+Pembrolizumab combination groups. The combination of FOLR1-TCB+TA-PD1-IL2v PQARK cleavable linker showed similar tumor growth inhibition as the combination of FOLR1-TCB+PD1-IL2v non masked group.

### DETAILED DESCRIPTION

### Definitions

Terms are used herein as generally used in the art, unless otherwise defined in the following.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 62.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 62). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as an IL-2 mutant peptide sequence, an IL-2 mutant polypeptide, IL-2 mutant protein or IL-2 mutant analog.

Designation of various forms of IL-2 is herein made with respect to the sequence shown in SEQ ID NO: 62. Various designations may be used herein to indicate the same mutation. For example a mutation from phenylalanine at position 42 to alanine can be indicated as 42A, A42, A42, F42A, or Phe42Ala.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g. an antibody chain) the wild-type form of this IL-2 mutant is IL-2 with a wild-type amino acid sequence fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine. In some embodiments wild-type IL-2 for the purpose of the present invention comprises the amino acid substitution C125A. In certain embodiments according to the invention the wild-type IL-2 polypeptide to which the mutant IL-2 polypeptide is compared comprises the amino acid sequence of SEQ ID NO: 62.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25.

The term “high-affinity IL-2 receptor” as used herein refers to the heterotrimeric form of the IL-2 receptor, consisting of the receptor γ-subunit (also known as common cytokine receptor γ-subunit, γ_{c}, or CD132), the receptor β-subunit (also known as CD122 or p70) and the receptor α-subunit (also known as CD25 or p55). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

By “regulatory T cell” or “T_{reg} cell” is meant a specialized type of CD4⁺ T cell that can suppress the responses of other T cells. T_{reg} cells are characterized by expression of the α-subunit of the IL-2 receptor (CD25) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. T_{reg} cells require IL-2 for their function and development and induction of their suppressive characteristics.

As used herein, the term “effector cells” refers to a population of lymphocytes that mediate the cytotoxic effects of IL-2. Effector cells include effector T cells such as CD8⁺ cytotoxic T cells, NK cells, lymphokine-activated killer (LAK) cells and macrophages/monocytes.

As used herein, the term “antigen binding molecule” refers in its broadest sense to a molecule that specifically binds an antigenic determinant. Examples of antigen binding molecules are immunoglobulins and derivatives, e.g., fragments, thereof.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g., a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “antigenic determinant” is synonymous with “antigen” and “epitope,” and refers to a site (e.g., a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be any native form of the proteins from any vertebrate source, including mammals such as primates (e.g., humans) and rodents (e.g., mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g., splice variants or allelic variants. The ability of an antigen binding moiety to bind to a specific antigenic determinant can be measured either through an enzyme-linked immunosorbent assay (ELISA) or other techniques familiar to one of skill in the art, e.g., surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers.

A “target cell antigen” as used herein refers to an antigenic determinant presented on the surface of a target cell, for example a cell in a tumor such as a cancer cell or a cell of the tumor stroma.

As used herein, the terms “first” and “second” with respect to antigen binding moieties etc., are used for convenience of distinguishing when there is more than one of each type of moiety. Use of these terms is not intended to confer a specific order or orientation of the protease-activatable IL-2 polypeptides or immunoconjugates unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

“TA” stands for tumor-activatable. “OA” stands for two-armed.

By “fused” is meant that the components (e.g., a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. Another term is single-chain variable fragments (scFv). In a particular such embodiment, the C-terminus of the Fab light chain is connected to the N-terminus of the Fab heavy chain in the single-chain Fab molecule.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g., γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region. The term “antibody” herein is used in the broadest sense and encompasses various antibody structures, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv), and single-domain antibodies. For a review of certain antibody fragments, see Hudson et al., Nat Med 9, 129-134 (2003). For a review of scFv fragments, see e.g., Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g., U.S. Pat. No. 6,248,516 B1). Antibody fragments can be made by various techniques, including but not limited to proteolytic digestion of an intact antibody as well as production by recombinant host cells (e.g., E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

The polypeptide sequences of the sequence listing are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

As used herein, the term “immunoconjugate” refers to a polypeptide molecule that includes at least one IL-2 moiety and at least one antigen binding moiety or effector cell binding moiety. In certain embodiments, the immunoconjugate comprises at least one IL-2 moiety, and at least two antigen binding moieties or at least two effector cell binding moieties. Particular immunoconjugates according to the invention essentially consist of one IL-2 moiety and two antigen binding moieties joined by one or more linker sequences. The antigen binding moiety can be joined to the IL-2 moiety by a variety of interactions and in a variety of configurations as described herein. Particular immunoconjugates according to the invention essentially consist of one IL-2 moiety and two effector cell binding moieties joined by one or more linker sequences. The effector cell binding moiety can be joined to the IL-2 moiety by a variety of interactions and in a variety of configurations as described herein.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g., the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g., 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program. The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term “nucleic acid molecule” refers to any one or more nucleic acid segments, e.g., DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g., ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g., PCT publication no. WO 2006/082515 or PCT publication no. WO 2012/130831).

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, protease-activatable IL-2 polypeptides or immunoconjugates of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

An “idiotype-specific polypeptide” as used herein refers to a polypeptide that recognizes the idiotype of an antigen-binding moiety, e.g., an antigen-binding moiety specific for CD3. The idiotype-specific polypeptide is capable of specifically binding to the variable region of the antigen-binding moiety and thereby reducing or preventing specific binding of the antigen-binding moiety to its cognate antigen. When associated with a molecule that comprises the antigen-binding moiety, the idiotype-specific polypeptide can function as a masking moiety of the molecule. Specifically disclosed herein are anti-idiotype antibodies or anti-idiotype-binding antibody fragments specific for the idiotype of anti-CD3 binding molecules.

“Protease” or “proteolytic enzyme” as used herein refers to any proteolytic enzyme that cleaves the linker at a recognition site and that is expressed by a target cell. Such proteases might be secreted by the target cell or remain associated with the target cell, e.g., on the target cell surface. Examples of proteases include but are not limited to metalloproteinases, e.g., matrix metalloproteinase 1-28 and A Disintegrin And Metalloproteinase (ADAM) 2, 7-12, 15, 17-23, 28-30 and 33, serine proteases, e.g., urokinase-type plasminogen activator and Matriptase, cysteine protease, aspartic proteases, and members of the cathepsin family.

“Protease activatable” as used herein, with respect to the interleukin-2 polypeptides, refers to an interleukin-2 polypeptides having reduced or abrogated ability to bind the interleukin-2 receptor due to a masking moiety that reduces or abrogates the interleukin-2 polypeptides's ability to bind to the interleukin-2 receptor. Upon dissociation of the masking moiety by proteolytic cleavage, e.g., by proteolytic cleavage of a linker connecting the masking moiety to the interleukin-2 polypeptide and or within the masking moiety, binding to the interleukin-2 receptor is restored and the interleukin-2 polypeptide is thereby activated.

“Reversibly concealing” as used herein refers to the binding of a masking moiety to an interleukin-2 polypeptide such as to prevent the interleukin-2 polypeptide from binding to its receptor. This concealing is reversible in that the masking moiety can be released from the interleukin-2 polypeptide, e.g. by protease cleavage, and thereby freeing the interleukin-2 polypeptide to bind to its receptor.

### EMBODIMENTS OF THE DISCLOSURE

In one embodiment, a protease-activatable interleukin-2 (IL-2) polypeptide is provided hat comprises (i) an IL-2 polypeptide, (ii) a masking moiety and (iii) a linker comprising a first protease cleavage site, wherein the linker has a length of 20 to 45 amino acids, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site. In a preferred embodiment, the linker has a length of 22 to 43 amino acids. In a preferred embodiment, the linker has a length of 25 to 38 amino acids. In a preferred embodiment, the linker has a length of 25. In another preferred embodiment, the linker has a length of 38 amino acids. In one embodiment, the masking moiety is covalently attached to the amino-terminus or carboxy-terminus of the interleukin-2 polypeptide through the linker. In one embodiment, the masking moiety is an IL-2 antagonist. In one embodiment, the masking moiety is an IL-2 antibody or an IL-2 receptor subunit. In one embodiment, the IL-2 antibody comprises a Fab molecule. In a preferred embodiment, the masking moiety is an antibody derived from MT204, preferably MT204. The MT204 antibody is disclosed e.g. in Volkland et al., Molecular Immunology 44 (2007) 1743-1753, and PCT-publication WO 2006/128690 A1. In a preferred embodiment, the masking moiety is a de-immunized MT204-derived binder. In a preferred embodiment, the MT204-derived masking moiety comprises a VL domain according to SEQ ID NO: 55 and a VH domain according to SEQ ID NO: 56. In one embodiment, the Fab molecule is a single-chain Fab molecule. In one embodiment, the second protease cleavage site is located between the variable domain of the heavy chain (VH) and the variable domain of the light chain (VL) of the single-chain Fab molecule. In one embodiment, the first protease cleavage site and the second protease cleavage site each comprise at least one protease recognition sequence.

In one embodiment, the protease recognition sequence of the first protease cleavage site and/or the protease recognition sequence of the second protease cleavage site is either YAARKGGI according to SEQ ID NO:60 and/or PQARK according to SEQ ID NO:61. In one embodiment, the protease recognition sequence of the first protease cleavage site is YAARKGGI according to SEQ ID NO:60 or PQARK according to SEQ ID NO:61. In one embodiment, the protease recognition sequence of the second protease cleavage site is YAARKGGI according to SEQ ID NO: 60 or PQARK according to SEQ ID NO:61. In one embodiment, the protease recognition sequence of the first protease cleavage site is YAARKGGI according to SEQ ID NO:60 and the protease recognition sequence of the second protease cleavage site is PQARK according to SEQ ID NO: 61. In one embodiment, the protease recognition sequence of the first protease cleavage site is PQARK according to SEQ ID NO:61 and the protease recognition sequence of the second protease cleavage site is YAARKGGI according to SEQ ID NO:60.

In one embodiment, the IL-2 polypeptide is a wild-type IL-2, preferably a human IL-2 according to SEQ ID NO: 62, or a mutant IL-2 polypeptide. In one embodiment, the mutant IL-2 polypeptide comprises any amino acid substitution selected from the group T3A, F42A, Y45A, L72G, C125A of human IL-2 according to SEQ ID NO:62. In one embodiment, the mutant IL-2 polypeptide comprises the amino acid substitutions F42A, Y45A and L72G of human IL-2 according to SEQ ID NO:62. In one embodiment, the mutant IL-2 polypeptide comprises the amino acid substitutions T3A, F42A, Y45A, L72G and C125A of human IL-2 according to SEQ ID NO: 62.

In one specific embodiment, the protease-activatable IL-2 polypeptide comprises an amino acid sequence that is at least about 95%, 96%, 97%, 98%, 99% or 100% identical to the sequence of SEQ ID NO: 27. In one specific embodiment, the protease-activatable IL-2 polypeptide comprises an amino acid sequence that is at least about 95%, 96%, 97%, 98%, 99% or 100% identical to the sequence of SEQ ID NO: 28. In one specific embodiment, the protease-activatable IL-2 polypeptide comprises an amino acid sequence that is at least about 95%, 96%, 97%, 98%, 99% or 100% identical to the sequence of SEQ ID NO: 29. In one specific embodiment, the protease-activatable IL-2 polypeptide comprises an amino acid sequence that is at least about 95%, 96%, 97%, 98%, 99% or 100% identical to the sequence of SEQ ID NO: 30.

In one specific embodiment, the protease-activatable IL-2 polypeptide comprises an amino acid sequence according to SEQ ID NO: 27. In one specific embodiment, the protease-activatable IL-2 polypeptide comprises an amino acid sequence according to SEQ ID NO: 28. In one specific embodiment, the protease-activatable IL-2 polypeptide comprises an amino acid sequence o according to SEQ ID NO: 29. In one specific embodiment, the protease-activatable IL-2 polypeptide comprises an amino acid sequence according to SEQ ID NO: 30.

In one embodiment, the IL-2 polypeptide is further attached to a non-IL-2 moiety. In one embodiment, the IL-2 polypeptide shares a carboxy-terminal peptide bond with the masking moiety and an amino-terminal peptide bond with the non-IL-2 moiety. In one embodiment, or the IL-2 polypeptide shares an amino-terminal peptide bond with the masking moiety and a carboxy-terminal peptide bond with the non-IL-2 moiety. In one embodiment the non-IL-2 moiety is an antigen binding moiety or an effector cell binding moiety.

### Immunoconjugates

In one embodiment, the invention provides an immunoconjugate comprising a protease-activatable IL-2 polypeptide as described herein and an antigen binding moiety and an effector cell binding moiety. In one embodiment, the invention provides an immunoconjugate comprising a protease-activatable IL-2 polypeptide as described herein and an antigen binding moiety. In one embodiment, the invention provides an immunoconjugate comprising a protease-activatable IL-2 polypeptide as described herein and an effector cell binding moiety.

In one embodiment, the protease-activatable IL-2 polypeptide shares an amino- or carboxy-terminal peptide bond with the antigen binding moiety or the effector cell binding moiety. In one embodiment, the immunoconjugate comprises a first and a second antigen binding moiety or a first and a second effector cell antigen binding moiety or an antigen binding moiety and an effector cell binding moiety. In one embodiment, (i) the protease-activatable IL-2 polypeptide shares an amino- or carboxy-terminal peptide bond with said first antigen binding moiety and said second antigen binding moiety shares an amino- or carboxy-terminal peptide bond with either a) said protease-activatable IL-2 polypeptide or b) said first antigen binding moiety; (ii) the protease-activatable IL-2 polypeptide shares an amino- or carboxy-terminal peptide bond with said first effector cell binding moiety and said second effector cell binding moiety shares an amino- or carboxy-terminal peptide bond with either a) said protease-activatable IL-2 polypeptide or b) said first effector cell binding moiety; (iii) the protease-activatable IL-2 polypeptide shares an amino- or carboxy-terminal peptide bond with the antigen binding moiety and the effector cell binding moiety shares an amino- or carboxy-terminal peptide bond with either a) said protease-activatable IL-2 polypeptide or b) said antigen binding moiety; or (iv) the protease-activatable IL-2 polypeptide shares an amino- or carboxy-terminal peptide bond with the effector cell binding moiety and the antigen binding moiety shares an amino- or carboxy-terminal peptide bond with either a) said protease-activatable IL-2 polypeptide or b) said effector cell binding moiety.

In one embodiment, the antigen binding moiety or the effector cell binding moiety comprised in the protease-activatable IL-2 polypeptide as disclosed herein or the immunoconjugate as disclosed herein is an antibody or an antibody fragment. In one embodiment, the antigen binding moiety and/or said effector cell binding moiety is selected from a Fab molecule and a scFv molecule. In one embodiment, the antigen binding moiety and/or said effector cell binding moiety is an immunoglobulin molecule, particularly an IgG molecule. In one embodiment, the antigen binding moiety is directed to an antigen presented on a tumor cell or in a tumor cell environment and/or wherein said effector cell binding moiety is directed to an effector cell present in a tumor cell environment in order to achieve cis-targeting.

In a specific embodiment, the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 23. In a specific embodiment, the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 24. In a specific embodiment, the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 25. In a specific embodiment, the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 26.

In one specific embodiment, the immunoconjugate comprises an amino acid sequence according to SEQ ID NO: 23. In one specific embodiment, the immunoconjugate comprises an amino acid sequence according to SEQ ID NO: 24. In one specific embodiment, the immunoconjugate comprises an amino acid sequence according to SEQ ID NO: 25. In one specific embodiment, the immunoconjugate comprises an amino acid sequence according to SEQ ID NO: 26.

In one embodiment, the immunoconjugate comprises an amino acid sequence according to SEQ ID NO: 5, an amino acid sequence according to SEQ ID NO: 22, and an amino acid sequence according to SEQ ID NO: 23. In one specific embodiment, the immunoconjugate comprises an amino acid sequence according to SEQ ID NO: 5, an amino acid sequence according to SEQ ID NO: 22, and an amino acid sequence according to SEQ ID NO: 24. In one specific embodiment, the immunoconjugate comprises an amino acid sequence according to SEQ ID NO: 5, an amino acid sequence according to SEQ ID NO: 22, and an amino acid sequence according to SEQ ID NO: 25. In one specific embodiment, the immunoconjugate comprises an amino acid sequence according to SEQ ID NO: 5, an amino acid sequence according to SEQ ID NO: 22, and an amino acid sequence according to SEQ ID NO: 26.

### Masking Moiety

The protease-activatable IL-2 polypeptide of the invention comprises at least one masking moiety. In one embodiment, the masking moiety masks the IL-2 polypeptide and comprises at least one of the heavy chain CDR1, the heavy chain CDR2, the heavy chain CDR3, the light chain CDR1, the light chain CDR2, and the light chain CDR3 of a MT204 derived Fab antibody. In a specific embodiment, the masking moiety masks the IL-2 polypeptide and comprises at least one of the heavy chain CDR1, the heavy chain CDR2, the heavy chain CDR3, the light chain CDR1, the light chain CDR2, and the light chain CDR3 of a MT204 derived Fab antibody with the amino acid sequence of SEQ ID NO: 55 and SEQ ID NO: 56. In one embodiment, the masking moiety comprises the heavy chain CDR1, the heavy chain CDR2, the heavy chain CDR3 as in the VH domain according to SEQ ID NO: 56, and the light chain CDR1, the light chain CDR2, and the light chain CDR3 as in the VL domain according to SEQ ID NO: 55. In a specific embodiment, the masking moiety that masks the IL-2 polypeptide comprises a VL domain according to SEQ ID NO: 55 and a VH domain according to SEQ ID NO: 56, wherein the masking moiety is a single-chain Fab molecule.

### Linkers

In one embodiment, the protease-activatable IL-2 polypeptide or the immunoconjugate comprises a linker having a protease recognition site comprising a polypeptide sequence that is at least about 95%, 96%, 97%, 98%, 99% or 100% identical to the sequence of SEQ ID NO: 63, 64, 65 or 66. In one specific embodiment, the protease recognition site comprises the polypeptide sequence of SEQ ID NO: 63, 64, 65 or 66.

### Polynucleotides

The invention further provides isolated polynucleotides encoding a protease-activatable IL-2 polypeptide or immunoconjugate as described herein or a fragment thereof.

The polynucleotides encoding protease-activatable IL-2 polypeptides or immunoconjugates of the invention may be expressed as a single polynucleotide that encodes the entire protease-activatable IL-2 polypeptides or immunoconjugates or as multiple (e.g., two or more) polynucleotides that are co-expressed. Polypeptides encoded by polynucleotides that are co-expressed may associate through, e.g., disulfide bonds or other means to form a functional protease-activatable IL-2 polypeptides or immunoconjugates. In the context of an immunoconjugate for example, the light chain portion of an antigen binding moiety may be encoded by a separate polynucleotide from the polynucleotide encoding the heavy chain of the immunoconjugate, an Fc domain subunit and optionally (part of) another antigen binding moiety. When co-expressed, the heavy chain polypeptides will associate with the light chain polypeptides to form the antigen binding moiety. In another example, the portion immunoconjugate comprising one of the two Fc domain subunits and optionally (part of) one or more antigen binding moieties could be encoded by a separate polynucleotide from the portion of the immunoconjugate comprising the other of the two Fc domain subunits and optionally (part of) an antigen binding moiety. When co-expressed, the Fc domain subunits will associate to form the Fc domain.

In some embodiments, the isolated polynucleotide encodes the entire immunoconjugate according to the invention as described herein. In other embodiments, the isolated polynucleotide encodes a polypeptides comprised in the immunoconjugate according to the invention as described herein.

In another embodiment, the present invention is directed to an isolated polynucleotide encoding a protease-activatable IL-2 polypeptide or immunoconjugate of the invention or a fragment 5 thereof. In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

The IL-2 polypeptide or immunoconjugate of the invention may be obtained, for example, by solid-state peptide synthesis (e.g., Merrifield solid phase synthesis) or recombinant production. For recombinant production one or more polynucleotide encoding the protease-activatable IL-2 polypeptide or immunoconjugate, e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a IL-2 polypeptide or immunoconjugate along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the protease-activatable IL-2 polypeptide or immunoconjugate (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the protease-activatable IL-2 polypeptide or immunoconjugate of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the IL-2 polypeptide or immunoconjugate is desired, DNA encoding a signal sequence may be placed upstream of the nucleic acid encoding a protease-activatable IL-2 polypeptide or immunoconjugate of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g., a histidine tag) or assist in labeling the protease-activatable IL-2 polypeptide or immunoconjugate may be included within or at the ends of the protease-activatable IL-2 polypeptide or immunoconjugate encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g., has been transformed or transfected with) a vector comprising a polynucleotide that encodes (part of) a protease-activatable IL-2 polypeptide or immunoconjugate of the invention. As used herein, the term “host cell” refers to any kind of cellular system which can be engineered to generate the protease-activatable IL-2 polypeptide or immunoconjugate of the invention or fragments thereof. Host cells suitable for replicating and for supporting expression of protease-activatable IL-2 polypeptides or immunoconjugates are well known in the art. Such cells may be transfected or transduced as appropriate with the particular expression vector and large quantities of vector containing cells can be grown for seeding large scale fermenters to obtain sufficient quantities of the IL-2 polypeptide or immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a protease-IL-2 polypeptide or immunoconjugate according to the invention is provided, wherein the method comprises culturing a host cell comprising a polynucleotide encoding the protease-activatable IL-2 polypeptide or immunoconjugate, as provided herein, under conditions suitable for expression of the protease-activatable IL-2 polypeptide or immunoconjugate, and recovering the protease-activatable IL-2 polypeptide or immunoconjugate from the host cell (or host cell culture medium).

The components of the protease-activatable IL-2 polypeptide or immunoconjugate are genetically fused to each other. Protease-activatable IL-2 polypeptides or immunoconjugates can be designed such that its components are fused directly to each other or indirectly through a linker sequence. The composition and length of the linker may be determined in accordance with methods well known in the art and may be tested for efficacy. Examples of linker sequences between different components of protease-activatable IL-2 polypeptides or immunoconjugates are found in the sequences provided herein. Additional sequences may also be included to incorporate a cleavage site to separate the individual components of the fusion if desired, for example an endopeptidase recognition sequence.

In certain embodiments the one or more antigen binding moieties of the immunoconjugates comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g., Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g., as described in U.S. Pat. No. 4,186,567) or can be obtained, for example, by screening combinatorial libraries comprising variable heavy chains and variable light chains (see e.g., U.S. Pat. No. 5,969,108 to McCafferty).

Any animal species of antibody, antibody fragment, antigen binding domain or variable region can be used in the immunoconjugates of the invention. Non-limiting antibodies, antibody fragments, antigen binding domains or variable regions useful in the present invention can be of murine, primate, or human origin. If the protease-activatable IL-2 polypeptide or immunoconjugate is intended for human use, a chimeric form of antibody may be used wherein the constant regions of the antibody are from a human. A “humanized” or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g., recipient antibody) framework and constant regions with or without retention of critical framework residues (e.g., those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31 (3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g., Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g., Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, NJ, 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the immunoconjugate of the invention to bind to a specific antigenic determinant can be measured either through an enzyme-linked immunosorbent assay (ELISA) or other techniques familiar to one of skill in the art, e.g., surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen. In certain embodiments, such a competing antibody binds to the same epitope (e.g., a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). Protease-activatable IL-2 polypeptides or immunoconjugate s prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the protease-activatable IL-2 polypeptide or immunoconjugate binds. For example, for affinity chromatography purification of protease-activatable IL-2 polypeptides or immunoconjugates of the invention, a matrix with protein A or protein G may be used. Sequential Protein A or G affinity chromatography and size exclusion chromatography can be used to isolate a protease-activatable IL-2 polypeptide or immunoconjugate. The purity of the protease-activatable IL-2 polypeptide or immunoconjugate can be determined by any of a variety of well-known analytical methods including gel electrophoresis, high pressure liquid chromatography, and the like.

### Assays

Protease-activatable IL-2 polypeptides or immunoconjugates provided herein may be identified, screened for, or characterized for their physical/chemical properties and/or biological activities by various assays known in the art.

### Affinity Assays

The affinity of the immunoconjugate for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of protease-activatable IL-2 polypeptides or immunoconjugates for different receptors or target antigens may be evaluated using cell lines expressing the particular receptor or target antigen, for example by flow cytometry (FACS). A specific illustrative and exemplary embodiment for measuring binding affinity is described in the following.

According to one embodiment, K_{D} is measured by surface plasmon resonance using a BIACORE® T100 machine (GE Healthcare) at 25 °C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 μg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25 °C at a flow rate of 30 μl/min for 120 s.

To determine the affinity to the target antigen, bispecific constructs are captured by an anti-human Fab specific antibody (GE Healthcare) that is immobilized on an activated CM5-sensor chip surface as described for the anti Penta-His antibody. The final amount of coupled protein is is approximately 12000 RU. The bispecific constructs are captured for 90 s at 300 nM. The target antigens are passed through the flow cells for 180 s at a concentration range from 250 to 1000 nM with a flowrate of 30 μl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

### Activity Assays

Biological activity of the protease-activatable IL-2 polypeptides or immunoconjugates of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

### Compositions, Formulations, and Routes of Administration

In a further aspect, the invention provides pharmaceutical compositions comprising any of the protease-activatable IL-2 polypeptides or immunoconjugates provided herein, e.g., for use in any of the below therapeutic methods. In one embodiment, a pharmaceutical composition comprises any of the protease-activatable IL-2 polypeptides or immunoconjugates provided herein and a pharmaceutically acceptable carrier. In another embodiment, a pharmaceutical composition comprises any of the protease-activatable IL-2 polypeptides or immunoconjugates provided herein and at least one additional therapeutic agent, e.g., as described below.

Further provided is a method of producing a protease-activatable IL-2 polypeptide or immunoconjugate of the invention in a form suitable for administration in vivo, the method comprising (a) obtaining a protease-activatable IL-2 polypeptide or immunoconjugate according to the invention, and (b) formulating the protease-activatable IL-2 polypeptide or immunoconjugate with at least one pharmaceutically acceptable carrier, whereby a preparation of protease-activatable IL-2 polypeptide or immunoconjugate is formulated for administration in vivo.

Pharmaceutical compositions of the present invention comprise a therapeutically effective amount of one or more protease-activatable IL-2 polypeptide or immunoconjugate dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one protease-activatable IL-2 polypeptide or immunoconjugate and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. Protease-activatable IL-2 polypeptides or immunoconjugates of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the protease-activatable IL-2 polypeptide or immunoconjugate of the invention.

Parenteral compositions include those designed for administration by injection, e.g., subcutaneous, intradermal, intralesional, intravenous, intraarterial intramuscular, intrathecal or intraperitoneal injection. For injection, the protease-activatable IL-2 polypeptides or immunoconjugates of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the protease-activatable IL-2 polypeptides or immunoconjugates may be in powder form for constitution with a suitable vehicle, e.g., sterile pyrogen-free water, before use. Sterile injectable solutions are prepared by incorporating the protease-activatable IL-2 polypeptides or immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g., films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the protease-activatable IL-2 polypeptides or immunoconjugates may also be formulated as a depot preparation. Such long acting formulations may be administered by implantation (for example subcutaneously or intramuscularly) or by intramuscular injection. Thus, for example, the protease-activatable IL-2 polypeptides or immunoconjugates may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the protease-activatable IL-2 polypeptides or immunoconjugates of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The protease-activatable IL-2 polypeptides or immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the protease-activatable IL-2 polypeptides or immunoconjugates provided herein may be used in therapeutic methods. Protease-activatable IL-2 polypeptides or immunoconjugates of the invention can be used as immunotherapeutic agents, for example in the treatment of cancers. For use in therapeutic methods, protease-activatable IL-2 polypeptides or immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, protease-activatable IL-2 polypeptides or immunoconjugates of the invention for use as a medicament are provided. In further aspects, protease-activatable IL-2 polypeptides or immunoconjugates of the invention for use in treating a disease are provided. In certain embodiments, protease-activatable IL-2 polypeptides or immunoconjugates of the invention for use in a method of treatment are provided. In one embodiment, the invention provides a protease-activatable IL-2 polypeptide or immunoconjugate as described herein for use in the treatment of a disease in an individual in need thereof. In certain embodiments, the invention provides a protease-activatable IL-2 polypeptide or immunoconjugate for use in a method of treating an individual having a disease comprising administering to the individual a therapeutically effective amount of the protease-activatable IL-2 polypeptide or immunoconjugate. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a protease-activatable IL-2 polypeptide or immunoconjugate as described herein for use in inducing lysis of a target cell, particularly a tumor cell. In certain embodiments, the invention provides a protease-activatable IL-2 polypeptide or immunoconjugate for use in a method of inducing lysis of a target cell, particularly a tumor cell, in an individual comprising administering to the individual an effective amount of the protease-activatable IL-2 polypeptide or immunoconjugate to induce lysis of a target cell. An “individual” according to any of the above embodiments is a mammal, preferably a human.

In a further aspect, the invention provides for the use of a protease-activatable IL-2 polypeptide or immunoconjugate of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a protease-activatable IL-2 polypeptide or immunoconjugate of the invention. In one embodiment a composition is administered to said individual, comprising the protease-activatable IL-2 polypeptide or immunoconjugate of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human.

In a further aspect, the invention provides a method for inducing lysis of a target cell, particularly a tumor cell.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a protease-activatable IL-2 polypeptide or immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the protease-activatable IL-2 polypeptide or immunoconjugate may not provide a cure but may only provide partial benefit. In some embodiments, a physiological change having some benefit is also considered therapeutically beneficial. Thus, in some embodiments, an amount of protease-activatable IL-2 polypeptide or immunoconjugate that provides a physiological change is considered an “effective amount” or a “therapeutically effective amount”. The subject, patient, or individual in need of treatment is typically a mammal, more specifically a human.

In some embodiments, an effective amount of a protease-activatable IL-2 polypeptide or immunoconjugate of the invention is administered to a cell. In other embodiments, a therapeutically effective amount of a protease-activatable IL-2 polypeptide or immunoconjugate of the invention is administered to an individual for the treatment of disease.

For the prevention or treatment of disease, the appropriate dosage of a protease-activatable IL-2 polypeptide or immunoconjugate of the invention (when used alone or in combination with one or more other additional therapeutic agents) will depend on the type of disease to be treated, the route of administration, the body weight of the patient, the type of IL-2 polypeptide or immunoconjugate, the severity and course of the disease, whether the IL-2 polypeptide or immunoconjugate is administered for preventive or therapeutic purposes, previous or concurrent therapeutic interventions, the patient's clinical history and response to the protease-activatable IL-2 polypeptide or immunoconjugate and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

A therapeutically effective dose of the protease-activatable IL-2 polypeptides or immunoconjugates described herein will generally provide therapeutic benefit without causing substantial toxicity. Toxicity and therapeutic efficacy of a protease-activatable IL-2 polypeptide or immunoconjugate can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Protease-activatable IL-2 polypeptides or immunoconjugates that exhibit large therapeutic indices are preferred. In one embodiment, the protease-activatable IL-2 polypeptide or immunoconjugate according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with protease-activatable IL-2 polypeptides or immunoconjugates of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The protease-activatable IL-2 polypeptides or immunoconjugates of the invention may be administered in combination with one or more other agents in therapy. For instance, a protease-activatable IL-2 polypeptide or immunoconjugate of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of protease-activatable IL-2 polypeptide or immunoconjugate used, the type of disorder or treatment, and other factors discussed above. The protease-activatable IL-2 polypeptide or immunoconjugate are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the protease-activatable IL-2 polypeptide or immunoconjugate of the invention can occur prior to, simultaneously, and/or following, administration of the additional therapeutic agent and/or adjuvant. Protease-activatable IL-2 polypeptides or immunoconjugates of the invention can also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a protease-activatable IL-2 polypeptide or immunoconjugate of the invention. The label or package insert indicates that the composition is used for treating the condition of choice. Moreover, the article of manufacture may comprise (a) a first container with a composition contained therein, wherein the composition comprises a protease-activatable IL-2 polypeptide or immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

SEQUENCES

| | SEQ ID | |
|---|---|---|
| Name | NO | aa |
| P1AH2050-hu IgG1 GL | 1, | DIQMTQSPSSLSASVGDRVTITCRASQGVGTNVGWYQQKPG |
| MT204_VL1- | C2184222326 | KAPKALIYSASFRYSGVPSRFSGSGSGTDFTLTISSLQPEDFAT |
| combo VH2 & VH6 PG | | YYCQQYYTYPYTFGGGTKVEIKRTVAAPSVFIFPPSDEQLKS |
| LALA-LC | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| P1AH2050-hu IgG1 GL | 2, | EVQLVESGGGLVQPGGSLRLSCAASGFTFSSYTLAWVRQAP |
| MT204_VL1- | C2184258141 | GKGLEWVAAIDSSSYTYSPDTVKGRFTISRDNSKNTLYLQM |
| combo VH2 & VH6 PG | | NSLRAEDTAVYYCARDSNWDALDYWGQGTTVTVSSASTKG |
| LALA-HC | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPEAAGGPSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALG |
| | | APIEKTISKAKGQPREPQVYTLPPSRDELTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPG |
| P1AH2051-hu IgG1 GL | 3, | DIQMTQSPSSLSASVGDRVTITCRASQGVGTNVGWYQQKPG |
| MT204_VL1a- | C2184258142 | KAPKALIYSASFLYSGVPSRFSGSGSGTDFTLTISSLQPEDFAT |
| combo VH2 & VH6 PG | | YYCQQYYTYPYTFGGGTKVEIKRTVAAPSVFIFPPSDEQLKS |
| LALA-LC | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| P1AH2051-hu IgG1 GL | identical to | EVQLVESGGGLVQPGGSLRLSCAASGFTFSSYTLAWVRQAP |
| MT204_VL1a- | #2 | GKGLEWVAAIDSSSYTYSPDTVKGRFTISRDNSKNTLYLQM |
| combo VH2 & VH6 PG | (C2184258141) | NSLRAEDTAVYYCARDSNWDALDYWGQGTTVTVSSASTKG |
| LALA-HC | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPEAAGGPSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALG |
| | | APIEKTISKAKGQPREPQVYTLPPSRDELTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPG |
| P1AH2052-hu IgG1 GL | 4, | DIQMTQSPSSLSASVGDRVTITCRASQGVGTNVGWYQQKPG |
| MT204_VL1b- | C2184258143 | KAPKLLIYSASFLYSGVPSRFSGSGSGTDFTLTISSLQPEDFAT |
| combo VH2 & VH6 PG | | YYCQQYYTYPYTFGGGTKVEIKRTVAAPSVFIFPPSDEQLKS |
| LALA-LC | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| P1AH2052-hu IgG1 GL | identical to | EVQLVESGGGLVQPGGSLRLSCAASGFTFSSYTLAWVRQAP |
| MT204_VL1b- | #2 | GKGLEWVAAIDSSSYTYSPDTVKGRFTISRDNSKNTLYLQM |
| combo VH2 & VH6 PG | (C2184258141) | NSLRAEDTAVYYCARDSNWDALDYWGQGTTVTVSSASTKG |
| LALA-HC | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPEAAGGPSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALG |
| | | APIEKTISKAKGQPREPQVYTLPPSRDELTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPG |
| P1AH4157-PD1- | 5, | DIVMTQSPDSLAVSLGERATINCKASESVDTSDNSFIHWYQQ |
| 376_VL2_VH4-LC | C2184254080 | KPGQSPKLLIYRASTLESGVPDRFSGSGSGTDFTLTISSLQAE |
| | | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AH4157-PD1- | 6, | EVQLLESGGGLVQPGGSLRLSCAASGFSFSSYTMSWVRQAP |
| 376_VL2_VH4-HC | C2184254074 | GKGLEWVSAISGGGRDIYYPDSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AH4158-PD1- | 7, | DIVMTQSPDSLAVSLGERATINCKASESVDTSDNSFIHWYQQ |
| 376_VL7_VH4-LC | C2184254078 | KPGQSPKLLIYRESTQESGVPDRFSGSGSGTDFTLTISSLQAE |
| | | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AH4158-PD1- | identical to | EVQLLESGGGLVQPGGSLRLSCAASGFSFSSYTMSWVRQAP |
| 376_VL7_VH4-HC | #6 | GKGLEWVSAISGGGRDIYYPDSVKGRFTISRDNSKNTLYLQ |
| | (C2184254074) | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AH4159-PD1- | 8, | DIVMTQSPDSLAVSLGERATINCKASESVDTSDNSFIHWYQQ |
| 376_VL2_7_VH4-LC | C2184262991 | KPGQSPKLLIYRASTQESGVPDRFSGSGSGTDFTLTISSLQAE |
| | | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AH4159-PD1- | identical to | EVQLLESGGGLVQPGGSLRLSCAASGFSFSSYTMSWVRQAP |
| 376_VL2_7_VH4-HC | #6 | GKGLEWVSAISGGGRDIYYPDSVKGRFTISRDNSKNTLYLQ |
| | (C2184254074) | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AH4160-PD1- | identical to | DIVMTQSPDSLAVSLGERATINCKASESVDTSDNSFIHWYQQ |
| 376_VL7_VH1_2_4-LC | #7 | KPGQSPKLLIYRESTQESGVPDRFSGSGSGTDFTLTISSLQAE |
| | (C2184254078) | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AH4160-PD1- | 9, | EVQLVESGGGLVQPGGSLRLSCAASGFTFSSYTMSWVRQAP |
| 376_VL7_VH1_2_4-HC | C2184262992 | GKGLEWVSAISGGGRDIYYPDSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AH4161-PD1- | identical to | DIVMTQSPDSLAVSLGERATINCKASESVDTSDNSFIHWYQQ |
| 376_VL2_VH1_2_3_4-LC | #5 | KPGQSPKLLIYRASTLESGVPDRFSGSGSGTDFTLTISSLQAE |
| | (C2184254080) | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AH4161-PD1- | 10 | EVQLVESGGGLVQPGGSLRLSCAASGFTFSSYAMSWVRQAP |
| 376_VL2_VH1_2_3_4-HC | (C2184262993) | GKGLEWVSAISGGGRDIYYPDSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI0356-PD1-1030- | 11, | DIVMTQSPDSLAVSLGERATINCKASESVDTSDNSFIHWYQQ |
| VH_158L_D62E-LC | C02011851 | KPGQSPKLLIYRSSTLESGVPDRFSGSGSGTDFTLTISSLQAED |
| | | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AI0356, PD1-1030- | 12, | EVQLLESGGGLVQPGGSLRLSCAASGFSFSSYTMSWVRQAP |
| VH_158L_D62E-HC | C2184267630 | GKGLEWVATISGGGRDLYYPESVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI0357-PD1-1030- | identical to | DIVMTQSPDSLAVSLGERATINCKASESVDTSDNSFIHWYQQ |
| VH_158T-LC | #11 | KPGQSPKLLIYRSSTLESGVPDRFSGSGSGTDFTLTISSLQAED |
| | (C02011851) | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AI0357-PD1-1030- | 13, | EVQLLESGGGLVQPGGSLRLSCAASGFSFSSYTMSWVRQAP |
| VH_158T-HC | C2184267631 | GKGLEWVATISGGGRDTYYPDSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI0358-PD1-1030- | identical to | DIVMTQSPDSLAVSLGERATINCKASESVDTSDNSFIHWYQQ |
| VH_158T_P61A-LC | #11 | KPGQSPKLLIYRSSTLESGVPDRFSGSGSGTDFTLTISSLQAED |
| | (C02011851) | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AI0358-PD1-1030- | 14, | EVQLLESGGGLVQPGGSLRLSCAASGFSFSSYTMSWVRQAP |
| VH_158T_P61A-HC | C2184267632 | GKGLEWVATISGGGRDTYYADSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI0359-PD1-1030- | identical to | DIVMTQSPDSLAVSLGERATINCKASESVDTSDNSFIHWYQQ |
| VH_P61A-LC | #11 | KPGQSPKLLIYRSSTLESGVPDRFSGSGSGTDFTLTISSLQAED |
| | (C02011851) | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AI0359-PD1-1030- | 15, | EVQLLESGGGLVQPGGSLRLSCAASGFSFSSYTMSWVRQAP |
| VH_P61A-HC | C2184267633 | GKGLEWVATISGGGRDIYYADSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI0360-PD1-1030- | identical to | DIVMTQSPDSLAVSLGERATINCKASESVDTSDNSFIHWYQQ |
| VH_Y60F_D62E-LC | #11 | KPGQSPKLLIYRSSTLESGVPDRFSGSGSGTDFTLTISSLQAED |
| | (C02011851) | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AI0360-PD1-1030- | 16, | EVQLLESGGGLVQPGGSLRLSCAASGFSFSSYTMSWVRQAP |
| VH_Y60F_D62E-HC | C2184267634 | GKGLEWVATISGGGRDIYFPESVKGRFTISRDNSKNTLYLQM |
| | | NSLRAEDTAVYYCVLLTGRVYFALDSWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPEAAGGPSVFLFPPKPK |
| | | DTLMISRTPEVTCVVVDVSHEDPEVKFNWYVDGVEVHNAK |
| | | TKPREEQYNSTYRVVSVLTVLHQDWLNGKEYKCKVSNKAL |
| | | GAPIEKTISKAKGQPREPQVYTLPPSRDELTKNQVSLTCLVK |
| | | GFYPSDIAVEWESNGQPENNYKTTPPVLDSDGSFFLYSKLTV |
| | | DKSRWQQGNVFSCSVMHEALHNHYTQKSLSLSPG |
| P1AI1648-PD1- | identical to | DIVMTQSPDSLAVSLGERATINCKASESVDTSDNSFIHWYQQ |
| 376_VL2_VH4_I58L_D62E | #5 | KPGQSPKLLIYRASTLESGVPDRFSGSGSGTDFTLTISSLQAE |
| IgG PG LALA-LC | (C2184254080 | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI1648-PD1- | 17, | EVQLLESGGGLVQPGGSLRLSCAASGFSFSSYTMSWVRQAP |
| 376_VL2_VH4_I58L_D62E | C2184269807 | GKGLEWVSAISGGGRDLYYPESVKGRFTISRDNSKNTLYLQ |
| IgG PG LALA-HC | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI1649-PD1- | identical to | DIVMTQSPDSLAVSLGERATINCKASESVDTSDNSFIHWYQQ |
| 376_VL2_VH4_I58T IgG | #5 | KPGQSPKLLIYRASTLESGVPDRFSGSGSGTDFTLTISSLQAE |
| PG LALA-LC | (C2184254080) | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI1649-PD1- | 18, | EVQLLESGGGLVQPGGSLRLSCAASGFSFSSYTMSWVRQAP |
| 376_VL2_VH4_I58T IgG | C2184269808 | GKGLEWVSAISGGGRDTYYPDSVKGRFTISRDNSKNTLYLQ |
| PG LALA-HC | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI1650-PD1- | identical to | DIVMTQSPDSLAVSLGERATINCKASESVDTSDNSFIHWYQQ |
| 376_VL2_VH4_I58T_P61A | #5 | KPGQSPKLLIYRASTLESGVPDRFSGSGSGTDFTLTISSLQAE |
| IgG PG LALA-LC | (C2184254080) | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI1650-PD1- | 19, | EVQLLESGGGLVQPGGSLRLSCAASGFSFSSYTMSWVRQAP |
| 376_VL2_VH4_I58T_P61A | C2184269809 | GKGLEWVSAISGGGRDTYYADSVKGRFTISRDNSKNTLYLQ |
| IgG PG LALA-HC | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI1651-PD1- | identical to | DIVMTQSPDSLAVSLGERATINCKASESVDTSDNSFIHWYQQ |
| 376_VL2_VH4_P61A IgG | #5 | KPGQSPKLLIYRASTLESGVPDRFSGSGSGTDFTLTISSLQAE |
| PG LALA-LC | (C2184254080) | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI1651-PD1- | 20, | EVQLLESGGGLVQPGGSLRLSCAASGFSFSSYTMSWVRQAP |
| 376_VL2_VH4_P61A IgG | C2184269810 | GKGLEWVSAISGGGRDIYYADSVKGRFTISRDNSKNTLYLQ |
| PG LALA-HC | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPG |
| P1AI1652-PD1- | identical to | DIVMTQSPDSLAVSLGERATINCKASESVDTSDNSFIHWYQQ |
| 376_VL2_VH4_Y60F_ | #5 | KPGQSPKLLIYRASTLESGVPDRFSGSGSGTDFTLTISSLQAE |
| D62E IgG PG LALA-LC | (C2184254080) | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI1652-PD1- | 21, | EVQLLESGGGLVQPGGSLRLSCAASGFSFSSYTMSWVRQAP |
| 376_VL2_VH4_Y60F_ | C2184269811 | GKGLEWVSAISGGGRDIYFPESVKGRFTISRDNSKNTLYLQM |
| D62E IgG PG LALA-HC | | NSLRAEDTAVYYCVLLTGRVYFALDSWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPEAAGGPSVFLFPPKPK |
| | | DTLMISRTPEVTCVVVDVSHEDPEVKFNWYVDGVEVHNAK |
| | | TKPREEQYNSTYRVVSVLTVLHQDWLNGKEYKCKVSNKAL |
| | | GAPIEKTISKAKGQPREPQVYTLPPSRDELTKNQVSLTCLVK |
| | | GFYPSDIAVEWESNGQPENNYKTTPPVLDSDGSFFLYSKLTV |
| | | DKSRWQQGNVFSCSVMHEALHNHYTQKSLSLSPG |
| P1AI4322-pro-PD1- | identical to | DIVMTQSPDSLAVSLGERATINCKASESVDTSDNSFIHWYQQ |
| IL2v_C- | #5 | KPGQSPKLLIYRASTLESGVPDRFSGSGSGTDFTLTISSLQAE |
| term_masked_PQARK_ | (C2184254080) | DVAVYYCQQNYDVPWTFGQGTKVEIKRTVAAPSVFIFPPSD |
| 38 mer-LC | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4322-pro-PD1- | 22, | EVQLLESGGGLVQPGGSLRLSCAASGFSFSSYTMSWVRQAP |
| IL2v_C- | C2184263076 | GKGLEWVSAISGGGRDIYYPDSVKGRFTISRDNSKNTLYLQ |
| term_masked_PQARK_ | | MNSLRAEDTAVYYCVLLTGRVYFALDSWGQGTLVTVSSAS |
| 38 mer-HC hole | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4322-pro-PD1- | 23, | EVQLLESGGGLVQPGGSLRLSCAASGFSFSSYTMSWVRQAP |
| IL2v_C- | C2184275090 | GKGLEWVSAISGGGRDIYYPDSVKGRFTISRDNSKNTLYLQ |
| term_masked_PQARK_ | | MNSLRAEDTAVYYCVLLTGRVYFALDSWGQGTLVTVSSAS |
| 38 mer-HC knob | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGSGGGGSGGPQARKGGSGGG |
| | | GSGGGGSGGGGSGGGGGEVQLVESGGGLVQPGGSLRLSCA |
| | | ASGFTFSSYTLAWVRQAPGKGLEWVAAIDSSSYTYSPDTVK |
| | | GRFTISRDNSKNTLYLQMNSLRAEDTAVYYCARDSNWDAL |
| | | DYWGQGTTVTVSSGGSGGGGPQARKGGSGGGGGDIQMTQS |
| | | PSSLSASVGDRVTITCRASQGVGTNVGWYQQKPGKAPKALI |
| | | YSASFLYSGVPSRFSGSGSGTDFTLTISSLQPEDFATYYCQQY |
| | | YTYPYTFGGGTKVEIK |
| P1AI4323-pro-PD1- | identical to | DIVMTQSPDSLAVSLGERATINCKASESVDTSDNSFIHWYQQ |
| IL2v_C- | #5 | KPGQSPKLLIYRASTLESGVPDRFSGSGSGTDFTLTISSLQAE |
| term_masked_PQARK_ | (C2184254080) | DVAVYYCQQNYDVPWTFGQGTKVEIKRTVAAPSVFIFPPSD |
| 25 mer-LC | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4323-pro-PD1- | identical to | EVQLLESGGGLVQPGGSLRLSCAASGFSFSSYTMSWVRQAP |
| IL2v_C- | #22 | GKGLEWVSAISGGGRDIYYPDSVKGRFTISRDNSKNTLYLQ |
| term_masked_PQARK_ | (C2184263076) | MNSLRAEDTAVYYCVLLTGRVYFALDSWGQGTLVTVSSAS |
| 25 mer-HC hole | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4323-pro-PD1- | 24, | EVQLLESGGGLVQPGGSLRLSCAASGFSFSSYTMSWVRQAP |
| IL2v_C- | C2184275091 | GKGLEWVSAISGGGRDIYYPDSVKGRFTISRDNSKNTLYLQ |
| term_masked_PQARK_ | | MNSLRAEDTAVYYCVLLTGRVYFALDSWGQGTLVTVSSAS |
| 25 mer-HC knob | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGSGGGGSGGPQARKGGSGGG |
| | | GSGGEVQLVESGGGLVQPGGSLRLSCAASGFTFSSYTLAWV |
| | | RQAPGKGLEWVAAIDSSSYTYSPDTVKGRFTISRDNSKNTLY |
| | | LQMNSLRAEDTAVYYCARDSNWDALDYWGQGTTVTVSSG |
| | | GSGGGGPQARKGGSGGGGGDIQMTQSPSSLSASVGDRVTIT |
| | | CRASQGVGTNVGWYQQKPGKAPKALIYSASFLYSGVPSRFS |
| | | GSGSGTDFTLTISSLQPEDFATYYCQQYYTYPYTFGGGTKVE |
| | | IK |
| P1AI4324-pro-PD1- | identical to | DIVMTQSPDSLAVSLGERATINCKASESVDTSDNSFIHWYQQ |
| IL2v_C- | #5 | KPGQSPKLLIYRASTLESGVPDRFSGSGSGTDFTLTISSLQAE |
| term_masked_YAARKGGI_ | (C2184254080) | DVAVYYCQQNYDVPWTFGQGTKVEIKRTVAAPSVFIFPPSD |
| 38 mer-LC | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4324-pro-PD1- | identical to | EVQLLESGGGLVQPGGSLRLSCAASGFSFSSYTMSWVRQAP |
| IL2v_C- | #22 | GKGLEWVSAISGGGRDIYYPDSVKGRFTISRDNSKNTLYLQ |
| term_masked_YAARKGGI_ | (C2184263076) | MNSLRAEDTAVYYCVLLTGRVYFALDSWGQGTLVTVSSAS |
| 38 mer-HC hole | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4324-pro-PD1- | 25, | EVQLLESGGGLVQPGGSLRLSCAASGFSFSSYTMSWVRQAP |
| IL2v_C- | C2184275092 | GKGLEWVSAISGGGRDIYYPDSVKGRFTISRDNSKNTLYLQ |
| term_masked_YAARKGGI_ | | MNSLRAEDTAVYYCVLLTGRVYFALDSWGQGTLVTVSSAS |
| 38 mer-HC knob | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGSGGGGSGGYAARKGGIGGSG |
| | | GGGSGGGGSGGGGSGGEVQLVESGGGLVQPGGSLRLSCAA |
| | | SGFTFSSYTLAWVRQAPGKGLEWVAAIDSSSYTYSPDTVKG |
| | | RFTISRDNSKNTLYLQMNSLRAEDTAVYYCARDSNWDALD |
| | | YWGQGTTVTVSSGGSGGGYAARKGGIGGSGGGDIQMTQSP |
| | | SSLSASVGDRVTITCRASQGVGTNVGWYQQKPGKAPKALIY |
| | | SASFLYSGVPSRFSGSGSGTDFTLTISSLQPEDFATYYCQQYY |
| | | TYPYTFGGGTKVEIK |
| P1AI4325-pro-PD1- | identical to | DIVMTQSPDSLAVSLGERATINCKASESVDTSDNSFIHWYQQ |
| IL2v_C- | #5 | KPGQSPKLLIYRASTLESGVPDRFSGSGSGTDFTLTISSLQAE |
| term_masked_YAARKGGI_ | (C2184254080) | DVAVYYCQQNYDVPWTFGQGTKVEIKRTVAAPSVFIFPPSD |
| 25 mer-LC | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4325-pro-PD1- | identical to | EVQLLESGGGLVQPGGSLRLSCAASGFSFSSYTMSWVRQAP |
| IL2v_C- | #22 | GKGLEWVSAISGGGRDIYYPDSVKGRFTISRDNSKNTLYLQ |
| term_masked_YAARKGGI_ | (C2184263076) | MNSLRAEDTAVYYCVLLTGRVYFALDSWGQGTLVTVSSAS |
| 25 mer-HC hole | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4325-pro-PD1- | 26, | EVQLLESGGGLVQPGGSLRLSCAASGFSFSSYTMSWVRQAP |
| IL2v_C- | C2184275093 | GKGLEWVSAISGGGRDIYYPDSVKGRFTISRDNSKNTLYLQ |
| term_masked_YAARKGGI_ | | MNSLRAEDTAVYYCVLLTGRVYFALDSWGQGTLVTVSSAS |
| 25 mer-HC knob | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGSGGGGSGGYAARKGGIGGSG |
| | | GGGEVQLVESGGGLVQPGGSLRLSCAASGFTFSSYTLAWVR |
| | | QAPGKGLEWVAAIDSSSYTYSPDTVKGRFTISRDNSKNTLYL |
| | | QMNSLRAEDTAVYYCARDSNWDALDYWGQGTTVTVSSGG |
| | | SGGGYAARKGGIGGSGGGDIQMTQSPSSLSASVGDRVTITCR |
| | | ASQGVGTNVGWYQQKPGKAPKALIYSASFLYSGVPSRFSGS |
| | | GSGTDFTLTISSLQPEDFATYYCQQYYTYPYTFGGGTKVEIK |
| IL2v_PQARK_38 mer- | 27 | APASSSTKKTQLQLEHLLLDLQMILNGINNYKNPKLTRMLTA |
| scFv mask with PQARK in | | KFAMPKKATELKHLQCLEEELKPLEEVLNGAQSKNFHLRPR |
| linker (as in P1AI4322) | | DLISNINVIVLELKGSETTFMCEYADETATIVEFLNRWITFAQ |
| | | SIISTLTGGSGGGGSGGPQARKGGSGGGGSGGGGSGGGGSG |
| | | GGGGEVQLVESGGGLVQPGGSLRLSCAASGFTFSSYTLAWV |
| | | RQAPGKGLEWVAAIDSSSYTYSPDTVKGRFTISRDNSKNTLY |
| | | LQMNSLRAEDTAVYYCARDSNWDALDYWGQGTTVTVSSG |
| | | GSGGGGPQARKGGSGGGGGDIQMTQSPSSLSASVGDRVTIT |
| | | CRASQGVGTNVGWYQQKPGKAPKALIYSASFLYSGVPSRFS |
| | | GSGSGTDFTLTISSLQPEDFATYYCQQYYTYPYTFGGGTKVE |
| | | IK |
| IL2v_PQARK_25 mer- | 28 | APASSSTKKTQLQLEHLLLDLQMILNGINNYKNPKLTRMLTA |
| scFv mask with PQARK in | | KFAMPKKATELKHLQCLEEELKPLEEVLNGAQSKNFHLRPR |
| linker (as in P1AI4323) | | DLISNINVIVLELKGSETTFMCEYADETATIVEFLNRWITFAQ |
| | | SIISTLTGGSGGGGSGGPQARKGGSGGGGSGGEVQLVESGG |
| | | GLVQPGGSLRLSCAASGFTFSSYTLAWVRQAPGKGLEWVA |
| | | AIDSSSYTYSPDTVKGRFTISRDNSKNTLYLQMNSLRAEDTA |
| | | VYYCARDSNWDALDYWGQGTTVTVSSGGSGGGGPQARKG |
| | | GSGGGGGDIQMTQSPSSLSASVGDRVTITCRASQGVGTNVG |
| | | WYQQKPGKAPKALIYSASFLYSGVPSRFSGSGSGTDFTLTISS |
| | | LQPEDFATYYCQQYYTYPYTFGGGTKVEIK |
| IL2v_YAARKGGI_38 mer- | 29 | APASSSTKKTQLQLEHLLLDLQMILNGINNYKNPKLTRMLTA |
| scFv mask with | | KFAMPKKATELKHLQCLEEELKPLEEVLNGAQSKNFHLRPR |
| YAARKGGI in linker (as | | DLISNINVIVLELKGSETTFMCEYADETATIVEFLNRWITFAQ |
| in P1AI4324) | | SIISTLTGGSGGGGSGGYAARKGGIGGSGGGGSGGGGSGGG |
| | | GSGGEVQLVESGGGLVQPGGSLRLSCAASGFTFSSYTLAWV |
| | | RQAPGKGLEWVAAIDSSSYTYSPDTVKGRFTISRDNSKNTLY |
| | | LQMNSLRAEDTAVYYCARDSNWDALDYWGQGTTVTVSSG |
| | | GSGGGYAARKGGIGGSGGGDIQMTQSPSSLSASVGDRVTITC |
| | | RASQGVGTNVGWYQQKPGKAPKALIYSASFLYSGVPSRFSG |
| | | SGSGTDFTLTISSLQPEDFATYYCQQYYTYPYTFGGGTKVEI |
| | | K |
| IL2v_YAARKGG_25 mer- | 30 | APASSSTKKTQLQLEHLLLDLQMILNGINNYKNPKLTRMLTA |
| scFv mask with | | KFAMPKKATELKHLQCLEEELKPLEEVLNGAQSKNFHLRPR |
| YAARKGGI in linker (as in | | DLISNINVIVLELKGSETTFMCEYADETATIVEFLNRWITFAQ |
| P1AI4325) | | SIISTLTGGSGGGGSGGYAARKGGIGGSGGGGEVQLVESGGG |
| | | LVQPGGSLRLSCAASGFTFSSYTLAWVRQAPGKGLEWVAAI |
| | | DSSSYTYSPDTVKGRFTISRDNSKNTLYLQMNSLRAEDTAVY |
| | | YCARDSNWDALDYWGQGTTVTVSSGGSGGGYAARKGGIG |
| | | GSGGGDIQMTQSPSSLSASVGDRVTITCRASQGVGTNVGWY |
| | | QQKPGKAPKALIYSASFLYSGVPSRFSGSGSGTDFTLTISSLQ |
| | | PEDFATYYCQQYYTYPYTFGGGTKVEIK |
| P1AF7506-OA MT204 | 31 | DIQMTQSPSSLSASVGDRVTITCKASQNVGTNVGWYQQKPG |
| IgG PG LALA-LC | (C22586796) | KAPKALIYSASFRYSGVPSRFSGSGSGTDFTLTISSLQPEDFAT |
| | | YYCQQYYTYPYTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| P1AF7506-OA MT204 | 32 | DKTHTCPPCPAPEAAGGPSVFLFPPKPKDTLMISRTPEVTCVV |
| IgG PG LALA-HC hole | (C01642558) | VDVSHEDPEVKFNWYVDGVEVHNAKTKPREEQYNSTYRVV |
| | | SVLTVLHQDWLNGKEYKCKVSNKALGAPIEKTISKAKGQPR |
| | | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHNHYTQKSLSLSPGK |
| P1AF7506-OA MT204 | 33 | EVQLVESGGGLVQPGGSLRLSCAASGFTFSSYTLAWVRQAP |
| IgG PG LALA-HC knob | (C2184222340) | GKGLEWVAAIDSSSYTYSPDTVRGRFTISRDNAKNSLYLQM |
| | | NSLRAEDTAVYYCARDSNWDALDYWGQGTTVTVSSASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPEAAGGPSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALG |
| | | APIEKTISKAKGQPREPQVYTLPPCRDELTKNQVSLWCLVKG |
| | | FYPSDIAVEWESNGQPENNYKTTPPVLDSDGSFFLYSKLTVD |
| | | KSRWQQGNVFSCSVMHEALHNHYTQKSLSLSPGK |
| P1AA6888-one shot | identical to | DIVMTQSPDSLAVSLGERATINCKASESVDTSDNSFIHWYQQ |
| humanization PD1-0103 (i.e. | #11 | KPGQSPKLLIYRSSTLESGVPDRFSGSGSGTDFTLTISSLQAED |
| PD1 IgG PG LALA, | (C02011851) | VAVYYCQQNYDVPWTFGQGTKVEIKRTVAAPSVFIFPPSDE |
| P1AD4476)-LC | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AA6888-one shot | 34 | EVQLLESGGGLVQPGGSLRLSCAASGFSFSSYTMSWVRQAP |
| humanization PD1-0103 (i.e. | (C01473218) | GKGLEWVATISGGGRDIYYPDSVKGRFTISRDNSKNTLYLQ |
| PD1 IgG PG LALA, | | MNSLRAEDTAVYYCVLLTGRVYFALDSWGQGTLVTVSSAS |
| P1AD4476)-HC | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPSRDELTKNQVSLTCL |
| | | VKGFYPSDIAVEWESNGQPENNYKTTPPVLDSDGSFFLYSKL |
| | | TVDKSRWQQGNVFSCSVMHEALHNHYTQKSLSLSPGK |
| P1AD9704-huPD1- | 35 | DKTHTCPPCPAPELLGGPSVFLFPPKPKDTLMISRTPEVTCVV |
| ECD_Fc-knob/hole, | (C02005019) | VDVSHEDPEVKFNWYVDGVEVHNAKTKPREEQYNSTYRVV |
| biotinylated (hole chain) | | SVLTVLHQDWLNGKEYKCKVSNKALPAPIEKTISKAKGQPR |
| | | EPQVCTLPPSRDELTKNQVSLSCAVKGFYPSDIAVEWESNGQ |
| | | PENNYKTTPPVLDSDGSFFLVSKLTVDKSRWQQGNVFSCSV |
| | | MHEALHNRFTQKSLSLSPGGSGLNDIFEAQKIEWHEARAHH |
| | | HHHH |
| P1AD9704-huPD1- | 36 | PGWFLDSPDRPWNPPTFSPALLVVTEGDNATFTCSFSNTSESF |
| ECD_Fc-knob/hole, | (C46296075) | VLNWYRMSPSNQTDKLAAFPEDRSQPGQDCRFRVTQLPNG |
| biotinylated (knob chain) | | RDFHMSVVRARRNDSGTYLCGAISLAPKAQIKESLRAELRVT |
| | | ERRAEVPTAHPSPSPRPAGQFQTLVGGGSDKTHTCPPCPAPE |
| | | LLGGPSVFLFPPKPKDTLMISRTPEVTCVVVDVSHEDPEVKF |
| | | NWYVDGVEVHNAKTKPREEQYNSTYRVVSVLTVLHQDWL |
| | | NGKEYKCKVSNKALPAPIEKTISKAKGQPREPQVYTLPPCRD |
| | | ELTKNQVSLWCLVKGFYPSDIAVEWESNGQPENNYKTTPPV |
| | | LDSDGSFFLYSKLTVDKSRWQQGNVFSCSVMHEALHNHYT |
| | | QKSLSLSPGSGLNDIFEAQKIEWHE |
| P1AG0879-hu IL2v G4S | 37 | APASSSTKKTQLQLEHLLLDLQMILNGINNYKNPKLTRMLTA |
| avi his | (C08901564) | KFAMPKKATELKHLQCLEEELKPLEEVLNGAQSKNFHLRPR |
| | | DLISNINVIVLELKGSETTFMCEYADETATIVEFLNRWITFAQ |
| | | SIISTLTGGGGSGLNDIFEAQKIEWHEARAHHHHHH |
| P1AA9690-MT204 IgG | Identical to | DIQMTQSPSSLSASVGDRVTITCKASQNVGTNVGWYQQKPG |
| PG LALA-LC | #31 | KAPKALIYSASFRYSGVPSRFSGSGSGTDFTLTISSLQPEDFAT |
| | (C22586796) | YYCQQYYTYPYTFGGGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| P1AA9690-MT204 IgG | 38 | EVQLVESGGGLVQPGGSLRLSCAASGFTFSSYTLAWVRQAP |
| PG LALA-HC | (C22587008) | GKGLEWVAAIDSSSYTYSPDTVRGRFTISRDNAKNSLYLQM |
| | | NSLRAEDTAVYYCARDSNWDALDYWGQGTTVTVSSASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKKVEPKSCDKTHTCPPCPAPEAAGGPSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALG |
| | | APIEKTISKAKGQPREPQVYTLPPSRDELTKNQVSLTCLVKGF |
| | | YPSDIAVEWESNGQPENNYKTTPPVLDSDGSFFLYSKLTVDK |
| | | SRWQQGNVFSCSVMHEALHNHYTQKSLSLSPGK |
| P1AE4422-PD1-IL2v-LC | identical to | DIVMTQSPDSLAVSLGERATINCKASESVDTSDNSFIHWYQQ |
| | #11 | KPGQSPKLLIYRSSTLESGVPDRFSGSGSGTDFTLTISSLQAED |
| | (C02011851) | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AE4422-PD1-IL2v- | 39 | EVQLLESGGGLVQPGGSLRLSCAASGFSFSSYTMSWVRQAP |
| HC hole | (C01483382) | GKGLEWVATISGGGRDIYYPDSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPGK |
| P1AE4422-PD1-IL2v-HC | 40 | EVQLLESGGGLVQPGGSLRLSCAASGFSFSSYTMSWVRQAP |
| knob | (C02597546 | GKGLEWVATISGGGRDIYYPDSVKGRFTISRDNSKNTLYLQ |
| | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGG |
| | | GSGGGGSGGGGSAPASSSTKKTQLQLEHLLLDLQMILNGINN |
| | | YKNPKLTRMLTAKFAMPKKATELKHLQCLEEELKPLEEVLN |
| | | GAQSKNFHLRPRDLISNINVIVLELKGSETTFMCEYADETATI |
| | | VEFLNRWITFAQSIISTLT |
| P1AA5355-FAP IL2v-LC | 41 | EIVLTQSPGTLSLSPGERATLSCRASQSVTSSYLAWYQQKPG |
| | (C01613867) | QAPRLLINVGSRRATGIPDRFSGSGSGTDFTLTISRLEPEDFAV |
| | | YYCQQGIMLPPTFGQGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| P1AA5355-FAP IL2v- | 42 | EVQLLESGGGLVQPGGSLRLSCAASGFTFSSYAMSWVRQAP |
| HC hole | (C01635489) | GKGLEWVSAIIGSGASTYYADSVKGRFTISRDNSKNTLYLQ |
| | | MNSLRAEDTAVYYCAKGWFGGFNYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPEAAGGPSVFLFPPKPK |
| | | DTLMISRTPEVTCVVVDVSHEDPEVKFNWYVDGVEVHNAK |
| | | TKPREEQYNSTYRVVSVLTVLHQDWLNGKEYKCKVSNKAL |
| | | GAPIEKTISKAKGQPREPQVCTLPPSRDELTKNQVSLSCAVK |
| | | GFYPSDIAVEWESNGQPENNYKTTPPVLDSDGSFFLVSKLTV |
| | | DKSRWQQGNVFSCSVMHEALHNHYTQKSLSLSPGK |
| P1AA5355-FAP IL2v-HC | 43 | EVQLLESGGGLVQPGGSLRLSCAASGFTFSSYAMSWVRQAP |
| knob | (C01635444) | GKGLEWVSAIIGSGASTYYADSVKGRFTISRDNSKNTLYLQ |
| | | MNSLRAEDTAVYYCAKGWFGGFNYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKKVEPKSCDKTHTCPPCPAPEAAGGPSVFLFPPKPK |
| | | DTLMISRTPEVTCVVVDVSHEDPEVKFNWYVDGVEVHNAK |
| | | TKPREEQYNSTYRVVSVLTVLHQDWLNGKEYKCKVSNKAL |
| | | GAPIEKTISKAKGQPREPQVYTLPPCRDELTKNQVSLWCLVK |
| | | GFYPSDIAVEWESNGQPENNYKTTPPVLDSDGSFFLYSKLTV |
| | | DKSRWQQGNVFSCSVMHEALHNHYTQKSLSLSPGGGGGSG |
| | | GGGSGGGGSAPASSSTKKTQLQLEHLLLDLQMILNGINNYK |
| | | NPKLTRMLTAKFAMPKKATELKHLQCLEEELKPLEEVLNGA |
| | | QSKNFHLRPRDLISNINVIVLELKGSETTFMCEYADETATIVE |
| | | FLNRWITFAQSIISTLT |
| P1AI4646-pro-PD1- | identical to | DIVMTQSPDSLAVSLGERATINCKASESVDTSDNSFIHWYQQ |
| IL2v_C-term_masked_non- | #5 | KPGQSPKLLIYRASTLESGVPDRFSGSGSGTDFTLTISSLQAE |
| cleavable_38 mer-LC | (C2184254080 | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4646-pro-PD1- | identical to | EVQLLESGGGLVQPGGSLRLSCAASGFSFSSYTMSWVRQAP |
| IL2v_C-term_masked_non- | #22 | GKGLEWVSAISGGGRDIYYPDSVKGRFTISRDNSKNTLYLQ |
| cleavable_38 mer-HC hole | (C2184263076) | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4646-pro-PD1- | 44 | EVQLLESGGGLVQPGGSLRLSCAASGFSFSSYTMSWVRQAP |
| IL2v_C-term_masked_non- | (C2184275466) | GKGLEWVSAISGGGRDIYYPDSVKGRFTISRDNSKNTLYLQ |
| cleavable_38 mer-HC knob | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGSGGGGSGGGGSGGGGSGGG |
| | | GSGGGGSGGGGSGGGGGEVQLVESGGGLVQPGGSLRLSCA |
| | | ASGFTFSSYTLAWVRQAPGKGLEWVAAIDSSSYTYSPDTVK |
| | | GRFTISRDNSKNTLYLQMNSLRAEDTAVYYCARDSNWDAL |
| | | DYWGQGTTVTVSSGGSGGGGSGGGGSGGGGSGGDIQMTQS |
| | | PSSLSASVGDRVTITCRASQGVGTNVGWYQQKPGKAPKALI |
| | | YSASFLYSGVPSRFSGSGSGTDFTLTISSLQPEDFATYYCQQY |
| | | YTYPYTFGGGTKVEIK |
| P1AI4647-pro-PD1- | identical to | DIVMTQSPDSLAVSLGERATINCKASESVDTSDNSFIHWYQQ |
| IL2v_C-term_masked_non- | #5 | KPGQSPKLLIYRASTLESGVPDRFSGSGSGTDFTLTISSLQAE |
| cleavable_25 mer-LC | (C2184254080 | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4647-pro-PD1- | identical to | EVQLLESGGGLVQPGGSLRLSCAASGFSFSSYTMSWVRQAP |
| IL2v_C-term_masked non- | #22 | GKGLEWVSAISGGGRDIYYPDSVKGRFTISRDNSKNTLYLQ |
| cleavable_25 mer-HC hole | (C2184263076) | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4647-pro-PD1- | 45 | EVQLLESGGGLVQPGGSLRLSCAASGFSFSSYTMSWVRQAP |
| IL2v_C-term_masked_non- | (C2184275467) | GKGLEWVSAISGGGRDIYYPDSVKGRFTISRDNSKNTLYLQ |
| cleavable_25 mer-HC knob | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGSGGGGSGGGGSGGGGSGGG |
| | | GSGGEVQLVESGGGLVQPGGSLRLSCAASGFTFSSYTLAWV |
| | | RQAPGKGLEWVAAIDSSSYTYSPDTVKGRFTISRDNSKNTLY |
| | | LQMNSLRAEDTAVYYCARDSNWDALDYWGQGTTVTVSSG |
| | | GSGGGGSGGGGSGGGGSGGDIQMTQSPSSLSASVGDRVTIT |
| | | CRASQGVGTNVGWYQQKPGKAPKALIYSASFLYSGVPSRFS |
| | | GSGSGTDFTLTISSLQPEDFATYYCQQYYTYPYTFGGGTKVE |
| | | IK |
| P1AI4648-pro-PD1- | identical to | DIVMTQSPDSLAVSLGERATINCKASESVDTSDNSFIHWYQQ |
| IL2v_C-term_non-masked- | #5 | KPGQSPKLLIYRASTLESGVPDRFSGSGSGTDFTLTISSLQAE |
| LC | (C2184254080 | DVAVYYCQQNYDVPWTFGQGTKVEIKRTVAAPSVFIFPPSD |
| | | EQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| P1AI4648-pro-PD1- | identical to | EVQLLESGGGLVQPGGSLRLSCAASGFSFSSYTMSWVRQAP |
| IL2v_C-term_non-masked- | #22 | GKGLEWVSAISGGGRDIYYPDSVKGRFTISRDNSKNTLYLQ |
| HC hole | (C2184263076) | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AI4648-pro-PD1- | 46 | EVQLLESGGGLVQPGGSLRLSCAASGFSFSSYTMSWVRQAP |
| IL2v_C-term_non-masked- | (C2184275468) | GKGLEWVSAISGGGRDIYYPDSVKGRFTISRDNSKNTLYLQ |
| HC knob | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGS |
| | | GGGGSGGGGSGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLT |
| P1AI4650-muPD1-0103- | 47 | KIVLTQSPASLPVSLGQRATISCRASESVDTSDNSFIHWYQQR |
| mIgG1-DAPG-DD/KK- | (C2184220339) | PGQSPKLLIYRSSTLESGVPARFSGSGSRTDFTLTIDPVEADD |
| IL2v aIL2 de-imm_MT204 | | VATYYCQQNYDVPWTFGGGTKLEIKRADAAPTVSIFPPSSEQ |
| scFv-YAARKGGI (in line | | LTSGGASVVCFLNNFYPKDINVKWKIDGSERQNGVLNSWTD |
| 38 mer on DD)-LC | | QDSKDSTYSMSSTLTLTKDEYERHNSYTCEATHKTSTSPIVK |
| | | SFNRNEC |
| P1AI4650-muPD1-0103- | 48 | EVILVESGGGLVKPGGSLKLSCAASGFSFSSYTMSWVRQTPE |
| mIgG1-DAPG-DD/KK- | (C2184220338) | KRLDWVATISGGGRDIYYPDSVKGRFTISRDNAKNTLYLEM |
| IL2v aIL2 de-imm_MT204 | | SSLMSEDTALYYCVLLTGRVYFALDSWGQGTSVTVSSAKTT |
| scFv-YAARKGGI (in line | | PPSVYPLAPGSAAQTNSMVTLGCLVKGYFPEPVTVTWNSGS |
| 38 mer on DD)-HC KK+ | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AI4650-muPD1-0103- | 49 | EVILVESGGGLVKPGGSLKLSCAASGFSFSSYTMSWVRQTPE |
| mIgG1-DAPG-DD/KK- | (C2184275470) | KRLDWVATISGGGRDIYYPDSVKGRFTISRDNAKNTLYLEM |
| IL2v aIL2 de-imm_MT204 | | SSLMSEDTALYYCVLLTGRVYFALDSWGQGTSVTVSSAKTT |
| scFv-YAARKGGI (in line | | PPSVYPLAPGSAAQTNSMVTLGCLVKGYFPEPVTVTWNSGS |
| 38 mer on DD)-HC DD− | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGSGGGGSGGGGSG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLTGGSGGGGSGGYAARKGGIGGSGGGGSGGGGSGG |
| | | GGSGGEVQLVESGGGLVQPGGSLRLSCAASGFTFSSYTLAW |
| | | VRQAPGKGLEWVAAIDSSSYTYSPDTVKGRFTISRDNSKNTL |
| | | YLQMNSLRAEDTAVYYCARDSNWDALDYWGQGTTVTVSS |
| | | GGSGGGYAARKGGIGGSGGGDIQMTQSPSSLSASVGDRVTIT |
| | | CRASQGVGTNVGWYQQKPGKAPKALIYSASFLYSGVPSRFS |
| | | GSGSGTDFTLTISSLQPEDFATYYCQQYYTYPYTFGGGTKVE |
| | | IK |
| P1AI4651-muPD1-0103- | identical to | KIVLTQSPASLPVSLGQRATISCRASESVDTSDNSFIHWYQQR |
| mIgG1-DAPG-DD/KK- | #47 | PGQSPKLLIYRSSTLESGVPARFSGSGSRTDFTLTIDPVEADD |
| IL2v aIL2 de-imm_MT204 | (C2184220339) | VATYYCQQNYDVPWTFGGGTKLEIKRADAAPTVSIFPPSSEQ |
| scFv-non-cleavable (in line | | LTSGGASVVCFLNNFYPKDINVKWKIDGSERQNGVLNSWTD |
| 38 mer on DD)-LC | | QDSKDSTYSMSSTLTLTKDEYERHNSYTCEATHKTSTSPIVK |
| | | SFNRNEC |
| P1AI4651-muPD1-0103- | identical to | EVILVESGGGLVKPGGSLKLSCAASGFSFSSYTMSWVRQTPE |
| mIgG1-DAPG-DD/KK- | #48 | KRLDWVATISGGGRDIYYPDSVKGRFTISRDNAKNTLYLEM |
| IL2v aIL2 de-imm_MT204 | (C2184220338) | SSLMSEDTALYYCVLLTGRVYFALDSWGQGTSVTVSSAKTT |
| scFv-non-cleavable (in line | | PPSVYPLAPGSAAQTNSMVTLGCLVKGYFPEPVTVTWNSGS |
| 38 mer on DD)-HC KK+ | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AI4651-muPD1-0103- | 50 | EVILVESGGGLVKPGGSLKLSCAASGFSFSSYTMSWVRQTPE |
| mIgG1-DAPG-DD/KK- | (C2184275471) | KRLDWVATISGGGRDIYYPDSVKGRFTISRDNAKNTLYLEM |
| IL2v aIL2 de-imm_MT204 | | SSLMSEDTALYYCVLLTGRVYFALDSWGQGTSVTVSSAKTT |
| scFv-non-cleavable (in line | | PPSVYPLAPGSAAQTNSMVTLGCLVKGYFPEPVTVTWNSGS |
| 38 mer on DD)-HC DD− | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGSGGGGSGGGGSG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLTGGSGGGGSGGGGSGGGGSGGGGSGGGGSGGGGS |
| | | GGGGGEVQLVESGGGLVQPGGSLRLSCAASGFTFSSYTLAW |
| | | VRQAPGKGLEWVAAIDSSSYTYSPDTVKGRFTISRDNSKNTL |
| | | YLQMNSLRAEDTAVYYCARDSNWDALDYWGQGTTVTVSS |
| | | GGSGGGGSGGGGSGGGGSGGDIQMTQSPSSLSASVGDRVTI |
| | | TCRASQGVGTNVGWYQQKPGKAPKALIYSASFLYSGVPSRF |
| | | SGSGSGTDFTLTISSLQPEDFATYYCQQYYTYPYTFGGGTKV |
| | | EIK |
| VL of parental PD-1 binder | 51 | DIVMTQSPDSLAVSLGERATINCKASESVDTSDNSFIHWYQQ |
| | | KPGQSPKLLIYRSSTLESGVPDRFSGSGSGTDFTLTISSLQAED |
| | | VAVYYCQQNYDVPWTFGQGTKVEIK |
| VH of parental PD-1 binder | 52 | EVQLLESGGGLVQPGGSLRLSCAASGFSFSSYTMSWVRQAP |
| | | GKGLEWVATISGGGRDIYYPDSVKGRFTISRDNSKNTLYLQ |
| | | MNSLRAEDTAVYYCVLLTGRVYFALDSWGQGTLVTVSS |
| VL of de-immunized PD-1 | 53 | DIVMTQSPDSLAVSLGERATINCKASESVDTSDNSFIHWYQQ |
| binder | | KPGQSPKLLIYRASTLESGVPDRFSGSGSGTDFTLTISSLQAE |
| | | DVAVYYCQQNYDVPWTFGQGTKVEIK |
| VH of de-immunized PD-1 | 54 | EVQLLESGGGLVQPGGSLRLSCAASGFSFSSYTMSWVRQAP |
| binder | | GKGLEWVSAISGGGRDIYYPDSVKGRFTISRDNSKNTLYLQ |
| | | MNSLRAEDTAVYYCVLLTGRVYFALDSWGQGTLVTVSS |
| VL of de-immunized | 55 | DIQMTQSPSSLSASVGDRVTITCRASQGVGTNVGWYQQKPG |
| MT204 | | KAPKALIYSASFLYSGVPSRFSGSGSGTDFTLTISSLQPEDFAT |
| | | YYCQQYYTYPYTFGGGTKVEIK |
| VH of de-immunized | 56 | EVQLVESGGGLVQPGGSLRLSCAASGFTFSSYTLAWVRQAP |
| MT204 | | GKGLEWVAAIDSSSYTYSPDTVKGRFTISRDNSKNTLYLQM |
| | | NSLRAEDTAVYYCARDSNWDALDYWGQGTTVTVSS |
| P1AG9606-PD1 IgG-IL2v | 57 | DIVMTQSPDSLAVSLGERATINCKASESVDTSDNSFIHWYQQ |
| aIL2 MT204 scFv-PQARK | | KPGQSPKLLIYRSSTLESGVPDRFSGSGSGTDFTLTISSLQAED |
| (in line on knob)-LC | | VAVYYCQQNYDVPWTFGQGTKVEIKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| P1AG9606-PD1 IgG-IL2v | 58 | EVQLLESGGGLVQPGGSLRLSCAASGFSFSSYTMSWVRQAP |
| aIL2 MT204 scFv-PQARK | | GKGLEWVATISGGGRDIYYPDSVKGRFTISRDNSKNTLYLQ |
| (in line on knob)-HC hole | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPG |
| P1AG9606-PD1 IgG-IL2v | 59 | EVQLLESGGGLVQPGGSLRLSCAASGFSFSSYTMSWVRQAP |
| aIL2 MT204 scFv-PQARK | | GKGLEWVATISGGGRDIYYPDSVKGRFTISRDNSKNTLYLQ |
| (in line on knob)-HC knob | | MNSLRAEDTAVYYCVLLTGRVYFALDSWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPEAAGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVYTLPPCRDELTKNQVSLWC |
| | | LVKGFYPSDIAVEWESNGQPENNYKTTPPVLDSDGSFFLYSK |
| | | LTVDKSRWQQGNVFSCSVMHEALHNHYTQKSLSLSPGGGG |
| | | GSGGGGSGGGGGAPASSSTKKTQLQLEHLLLDLQMILNGIN |
| | | NYKNPKLTRMLTAKFAMPKKATELKHLQCLEEELKPLEEVL |
| | | NGAQSKNFHLRPRDLISNINVIVLELKGSETTFMCEYADETA |
| | | TIVEFLNRWITFAQSIISTLTGGGGSGGGGSPQARKGGGGSG |
| | | GGGSGGGGSGGGGSGGGEVQLVESGGGLVQPGGSLRLSCA |
| | | ASGFTFSSYTLAWVRQAPGKGLEWVAAIDSSSYTYSPDTVR |
| | | GRFTISRDNAKNSLYLQMNSLRAEDTAVYYCARDSNWDAL |
| | | DYWGQGTTVTVSSGGGGSGGPQARKGGGGSGGGDIQMTQS |
| | | PSSLSASVGDRVTITCKASQNVGTNVGWYQQKPGKAPKALI |
| | | YSASFRYSGVPSRFSGSGSGTDFTLTISSLQPEDFATYYCQQY |
| | | YTYPYTFGGGTKVEIK |
| YAARKGGI protease | 60 | YAARKGGI |
| cleavage site | | |
| PQARK cleavage site | 61 | PQARK |
| human IL-2 wild type | 62 | APTSSSTKKTQLQLEHLLLDLQMILNGINNYKNPKLTRMLTF |
| | | KFYMPKKATELKHLQCLEEELKPLEEVLNLAQSKNFHLRPR |
| | | DLISNINVIVLELKGSETTFMCEYADETATIVEFLNRWITFCQ |
| | | SIISTLT |
| PQARK 38 mer linker | 63 | GGSGGGGSGGPQARKGGSGGGGSGGGGSGGGGSGGGGG |
| PQARK 25 mer linker | 64 | GGSGGGGSGGPQARKGGSGGGGSGG |
| YAARKGGI 38 mer linker | 65 | GGSGGGGSGGYAARKGGIGGSGGGGSGGGGSGGGGSGG |
| YAARKGGI 25 mer linker | 66 | GGSGGGGSGGYAARKGGIGGSGGGG |
| human IL2v | 67 | APASSSTKKTQLQLEHLLLDLQMILNGINNYKNPKLTRMLTA |
| | | KFAMPKKATELKHLQCLEEELKPLEEVLNGAQSKNFHLRPR |
| | | DLISNINVIVLELKGSETTFMCEYADETATIVEFLNRWITFAQ |
| | | SIISTLT |
| LCDR 1 of de-immunized | 68 | KASESVDTSDNSFIH |
| PD-1 binder | | |
| LCDR 2 of de-immunized | 69 | RASTLES |
| PD-1 binder | | |
| LCDR 3 of de-immunized | 70 | QQNYDVPWT |
| PD-1 binder | | |
| HCDR 1 of de-immunized | 71 | SYTMS |
| PD-1 binder | | |
| HCDR 2 of de-immunized | 72 | AISGGGRDIYYPDSVKG |
| PD-1 binder | | |
| HCDR 3 of de-immunized | 73 | LTGRVYFALDS |
| PD-1 binder | | |
| LCDR 1 of de-immunized | 74 | RASQGVGTNVG |
| MT204 | | |
| LCDR 2 of de-immunized | 75 | SASFLYS |
| MT204 | | |
| LCDR 3 of de-immunized | 76 | QQYYTYPYT |
| MT204 | | |
| HCDR 1 of de-immunized | 77 | SYTLA |
| MT204 | | |
| HCDR 2 of de-immunized | 78 | AIDSSSYTYSPDTVKG |
| MT204 | | |
| HCDR 3 of de-immunized | 79 | DSNWDALDY |
| MT204 | | |
| P1AK1120, FolR1tcb-D- | 80 | QAVVTQEPSLTVSPGGTVTLTCGSSTGAVTTSNYANWVQEK |
| 16D5-02 sf W(2)-common | (C08611380) | PGQAFRGLIGGTNKRAPGTPARFSGSLLGGKAALTLSGAQPE |
| LC | | DEAEYYCALWYSNLWVFGGGTKLTVLGQPKAAPSVTLFPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECS |
| P1AK1120, FolR1tcb-D- | 81 | EVQLLESGGGLVQPGGSLRLSCAASGFTFSSYAMNWVRQAP |
| 16D5-02 sf W(2)-HC knob | (C2184192521) | GKGLEWVSRIRSKYNNYATYYADSVKGRFTISRDDSKNTLY |
| | | LQMNSLRAEDTAVYYCVRASNFPASYVSYFAYWGQGTLVT |
| | | VSSASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKKVEPKSCDGGGGSGGGGSEVQLVESG |
| | | GGLVKPGGSLRLSCAASGFTFSNAWMSWVRQAPGKGLEWV |
| | | GRIKSKTDGGTTDYAAPVKGRFTISRDDSKNTLYLQMNSLK |
| | | TEDTAVYYCTTPWEWSWYDYWGQGTLVTVSSASTKGPSVF |
| | | PLAPSSKSTSGGTAALGCLVKDYFPEPVTVSWNSGALTSGV |
| | | HTFPAVLQSSGLYSLSSVVTVPSSSLGTQTYICNVNHKPSNTK |
| | | VDKKVEPKSCDKTHTCPPCPAPEAAGGPSVFLFPPKPKDTLM |
| | | ISRTPEVTCVVVDVSHEDPEVKFNWYVDGVEVHNAKTKPRE |
| | | EQYNSTYRVVSVLTVLHQDWLNGKEYKCKVSNKALGAPIE |
| | | KTISKAKGQPREPQVYTLPPCRDELTKNQVSLWCLVKGFYPS |
| | | DIAVEWESNGQPENNYKTTPPVLDSDGSFFLYSKLTVDKSR |
| | | WQQGNVFSCSVMHEALHNHYTQKSLSLSPGK |
| P1AK1120, FolR1tcb-D- | 82 | EVQLVESGGGLVKPGGSLRLSCAASGFTFSNAWMSWVRQA |
| 16D5-02 sf W(2)-HC hole | (C140693647) | PGKGLEWVGRIKSKTDGGTTDYAAPVKGRFTISRDDSKNTL |
| | | YLQMNSLKTEDTAVYYCTTPWEWSWYDYWGQGTLVTVSS |
| | | ASTKGPSVFPLAPSSKSTSGGTAALGCLVKDYFPEPVTVSWN |
| | | SGALTSGVHTFPAVLQSSGLYSLSSVVTVPSSSLGTQTYICNV |
| | | NHKPSNTKVDKKVEPKSCDKTHTCPPCPAPEAAGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSN |
| | | KALGAPIEKTISKAKGQPREPQVCTLPPSRDELTKNQVSLSCA |
| | | VKGFYPSDIAVEWESNGQPENNYKTTPPVLDSDGSFFLVSKL |
| | | TVDKSRWQQGNVFSCSVMHEALHNHYTQKSLSLSPGK |
| P1AK3638, muPD1-0103- | 83 | KIVLTQSPASLPVSLGQRATISCRASESVDTSDNSFIHWYQQR |
| mIgG1-DAPG-DD/KK- | (C2184220339) | PGQSPKLLIYRSSTLESGVPARFSGSGSRTDFTLTIDPVEADD |
| IL2v aIL2 de-imm_MT204 | | VATYYCQQNYDVPWTFGGGTKLEIKRADAAPTVSIFPPSSEQ |
| scFv-PQARK (in line | | LTSGGASVVCFLNNFYPKDINVKWKIDGSERQNGVLNSWTD |
| 25 mer on DD)-LC | | QDSKDSTYSMSSTLTLTKDEYERHNSYTCEATHKTSTSPIVK |
| | | SFNRNEC |
| P1AK3638, muPD1-0103- | 84 | EVILVESGGGLVKPGGSLKLSCAASGFSFSSYTMSWVRQTPE |
| mIgG1-DAPG-DD/KK- | (C2184306394) | KRLDWVATISGGGRDIYYPDSVKGRFTISRDNAKNTLYLEM |
| IL2v aIL2 de-imm_MT204 | | SSLMSEDTALYYCVLLTGRVYFALDSWGQGTSVTVSSAKTT |
| scFv-PQARK (in line | | PPSVYPLAPGSAAQTNSMVTLGCLVKGYFPEPVTVTWNSGS |
| 25 mer on DD)-HC DD− | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGSGGGGSGGGGSG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLTGGSGGGGSGGPQARKGGSGGGGSGGEVQLVESG |
| | | GGLVQPGGSLRLSCAASGFTFSSYTLAWVRQAPGKGLEWV |
| | | AAIDSSSYTYSPDTVKGRFTISRDNSKNTLYLQMNSLRAEDT |
| | | AVYYCARDSNWDALDYWGQGTTVTVSSGGSGGGGPQARK |
| | | GGSGGGGGDIQMTQSPSSLSASVGDRVTITCRASQGVGTNV |
| | | GWYQQKPGKAPKALIYSASFLYSGVPSRFSGSGSGTDFTLTIS |
| | | SLQPEDFATYYCQQYYTYPYTFGGGTKVEIK |
| P1AK3638, muPD1-0103- | 85 | EVILVESGGGLVKPGGSLKLSCAASGFSFSSYTMSWVRQTPE |
| mIgG1-DAPG-DD/KK- | (C2184220 | KRLDWVATISGGGRDIYYPDSVKGRFTISRDNAKNTLYLEM |
| IL2v aIL2 de-imm_MT204 | 338) | SSLMSEDTALYYCVLLTGRVYFALDSWGQGTSVTVSSAKTT |
| scFv-PQARK (in line | | PPSVYPLAPGSAAQTNSMVTLGCLVKGYFPEPVTVTWNSGS |
| 25 mer on DD)- HC KK+ | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AK3640, muPD1-0103- | identical to | KIVLTQSPASLPVSLGQRATISCRASESVDTSDNSFIHWYQQR |
| mIgG1-DAPG-DD/KK- | #83 | PGQSPKLLIYRSSTLESGVPARFSGSGSRTDFTLTIDPVEADD |
| IL2v aIL2 de-imm_MT204 | (C2184220 | VATYYCQQNYDVPWTFGGGTKLEIKRADAAPTVSIFPPSSEQ |
| scFv-non-cleavable (in line | 339) | LTSGGASVVCFLNNFYPKDINVKWKIDGSERQNGVLNSWTD |
| 25 mer on DD)-LC | | QDSKDSTYSMSSTLTLTKDEYERHNSYTCEATHKTSTSPIVK |
| | | SFNRNEC |
| P1AK3640, muPD1-0103- | 86 | EVILVESGGGLVKPGGSLKLSCAASGFSFSSYTMSWVRQTPE |
| mIgG1-DAPG-DD/KK- | (C2184306395) | KRLDWVATISGGGRDIYYPDSVKGRFTISRDNAKNTLYLEM |
| IL2v aIL2 de-imm_MT204 | | SSLMSEDTALYYCVLLTGRVYFALDSWGQGTSVTVSSAKTT |
| scFv-non-cleavable (in line | | PPSVYPLAPGSAAQTNSMVTLGCLVKGYFPEPVTVTWNSGS |
| 25 mer on DD)-HC DD− | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGSGGGGSGGGGSG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLTGGSGGGGSGGGGSGGGGGGGGSGGEVQLVESG |
| | | GGLVQPGGSLRLSCAASGFTFSSYTLAWVRQAPGKGLEWV |
| | | AAIDSSSYTYSPDTVKGRFTISRDNSKNTLYLQMNSLRAEDT |
| | | AVYYCARDSNWDALDYWGQGTTVTVSSGGSGGGGSGGGG |
| | | SGGGGSGGDIQMTQSPSSLSASVGDRVTITCRASQGVGTNV |
| | | GWYQQKPGKAPKALIYSASFLYSGVPSRFSGSGSGTDFTLTIS |
| | | SLQPEDFATYYCQQYYTYPYTFGGGTKVEIK |
| P1AK3640, muPD1-0103- | identical to | EVILVESGGGLVKPGGSLKLSCAASGFSFSSYTMSWVRQTPE |
| mIgG1-DAPG-DD/KK- | #85 | KRLDWVATISGGGRDIYYPDSVKGRFTISRDNAKNTLYLEM |
| IL2v aIL2 de-imm_MT204 | (C2184220338) | SSLMSEDTALYYCVLLTGRVYFALDSWGQGTSVTVSSAKTT |
| scFv-non-cleavable (in line | | PPSVYPLAPGSAAQTNSMVTLGCLVKGYFPEPVTVTWNSGS |
| 25 mer on DD)-HC KK+ | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AK3641, ratPD1- | 87 | DIVMTQGTLPNPVPSGESVSITCRSSKSLLYSDGKTYLNWYL |
| mIgG1-DAPG-DD/KK- | (C01486562) | QRPGQSPQLLIYWMSTRASGVSDRFSGSGSGTDFTLKISGVE |
| IL2v aIL2 de-imm_MT204 | | AEDVGIYYCQQGLEFPTFGGGTKLELKRADAAPTVSIFPPSSE |
| scFv-PQARK (in line | | QLTSGGASVVCFLNNFYPKDINVKWKIDGSERQNGVLNSWT |
| 25 mer on DD)-LC | | DQDSKDSTYSMSSTLTLTKDEYERHNSYTCEATHKTSTSPIV |
| | | KSFNRNEC |
| P1AK3641, ratPD1- | 88 | EVQLQESGPGLVKPSQSLSLTCSVTGYSITSSYRWNWIRKFP |
| mIgG1-DAPG-DD/KK- | (C2184306396) | GNRLEWMGYINSAGISNYNPSLKRRISITRDTSKNQFFLQVN |
| IL2v aIL2 de-imm_MT204 | | SVTTEDAATYYCARSDNMGTTPFTYWGQGTLVTVSSAKTTP |
| scFv-PQARK (in line | | PSVYPLAPGSAAQTNSMVTLGCLVKGYFPEPVTVTWNSGSL |
| 25 mer on DD)-HC DD− | | SSGVHTFPAVLQSDLYTLSSSVTVPSSTWPSQTVTCNVAHPA |
| | | SSTKVDKKIVPRDCGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGSGGGGSGGGGSG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLTGGSGGGGSGGPQARKGGSGGGGSGGEVQLVESG |
| | | GGLVQPGGSLRLSCAASGFTFSSYTLAWVRQAPGKGLEWV |
| | | AAIDSSSYTYSPDTVKGRFTISRDNSKNTLYLQMNSLRAEDT |
| | | AVYYCARDSNWDALDYWGQGTTVTVSSGGSGGGGPQARK |
| | | GGSGGGGGDIQMTQSPSSLSASVGDRVTITCRASQGVGTNV |
| | | GWYQQKPGKAPKALIYSASFLYSGVPSRFSGSGSGTDFTLTIS |
| | | SLQPEDFATYYCQQYYTYPYTFGGGTKVEIK |
| P1AK3641, ratPD1- | 89 | EVQLQESGPGLVKPSQSLSLTCSVTGYSITSSYRWNWIRKFP |
| mIgG1-DAPG-DD/KK- | (C2184220341) | GNRLEWMGYINSAGISNYNPSLKRRISITRDTSKNQFFLQVN |
| IL2v aIL2 de-imm_MT204 | | SVTTEDAATYYCARSDNMGTTPFTYWGQGTLVTVSSAKTTP |
| scFv-PQARK (in line | | PSVYPLAPGSAAQTNSMVTLGCLVKGYFPEPVTVTWNSGSL |
| 25 mer on DD)-HC KK+ | | SSGVHTFPAVLQSDLYTLSSSVTVPSSTWPSQTVTCNVAHPA |
| | | SSTKVDKKIVPRDCGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AK3649, ratPD1- | identical to | DIVMTQGTLPNPVPSGESVSITCRSSKSLLYSDGKTYLNWYL |
| mIgG1-DAPG-DD/KK- | #87 | QRPGQSPQLLIYWMSTRASGVSDRFSGSGSGTDFTLKISGVE |
| IL2v aIL2 de-imm_MT204 | (C01486562) | AEDVGIYYCQQGLEFPTFGGGTKLELKRADAAPTVSIFPPSSE |
| scFv-non-cleavable (in line | | QLTSGGASVVCFLNNFYPKDINVKWKIDGSERQNGVLNSWT |
| 25 mer on DD)-LC | | DQDSKDSTYSMSSTLTLTKDEYERHNSYTCEATHKTSTSPIV |
| | | KSFNRNEC |
| P1AK3649, ratPD1- | 90 | EVQLQESGPGLVKPSQSLSLTCSVTGYSITSSYRWNWIRKFP |
| mIgG1-DAPG-DD/KK- | (C2184306398) | GNRLEWMGYINSAGISNYNPSLKRRISITRDTSKNQFFLQVN |
| IL2v aIL2 de-imm_MT204 | | SVTTEDAATYYCARSDNMGTTPFTYWGQGTLVTVSSAKTTP |
| scFv-non-cleavable (in line | | PSVYPLAPGSAAQTNSMVTLGCLVKGYFPEPVTVTWNSGSL |
| 25 mer on DD)-HC DD− | | SSGVHTFPAVLQSDLYTLSSSVTVPSSTWPSQTVTCNVAHPA |
| | | SSTKVDKKIVPRDCGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGSGGGGSGGGGSG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLTGGSGGGGSGGGGSGGGGSGGGGSGGEVQLVESG |
| | | GGLVQPGGSLRLSCAASGFTFSSYTLAWVRQAPGKGLEWV |
| | | AAIDSSSYTYSPDTVKGRFTISRDNSKNTLYLQMNSLRAEDT |
| | | AVYYCARDSNWDALDYWGQGTTVTVSSGGSGGGGSGGGG |
| | | SGGGGSGGDIQMTQSPSSLSASVGDRVTITCRASQGVGTNV |
| | | GWYQQKPGKAPKALIYSASFLYSGVPSRFSGSGSGTDFTLTIS |
| | | SLQPEDFATYYCQQYYTYPYTFGGGTKVEIK |
| P1AK3649, ratPD1- | identical to | EVQLQESGPGLVKPSQSLSLTCSVTGYSITSSYRWNWIRKFP |
| mIgG1-DAPG-DD/KK- | #89 | GNRLEWMGYINSAGISNYNPSLKRRISITRDTSKNQFFLQVN |
| IL2v aIL2 de-imm_MT204 | (C2184220341) | SVTTEDAATYYCARSDNMGTTPFTYWGQGTLVTVSSAKTTP |
| scFv-non-cleavable (in line | | PSVYPLAPGSAAQTNSMVTLGCLVKGYFPEPVTVTWNSGSL |
| 25 mer on DD)-HC KK+ | | SSGVHTFPAVLQSDLYTLSSSVTVPSSTWPSQTVTCNVAHPA |
| | | SSTKVDKKIVPRDCGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AD4006, | 91 | DIVMTQGTLPNPVPSGESVSITCRSSKSLLYSDGKTYLNWYL |
| <muPD1>RMP1- | (C01686685) | QRPGQSPQLLIYWMSTRASGVSDRFSGSGSGTDFTLKISGVE |
| 14muIgG2akappaPG LALA- | | AEDVGIYYCQQGLEFPTFGGGTKLELKRTDAAPTVSIFPPSSE |
| LC | | QLTSGGASVVCFLNNFYPKDINVKWKIDGSERQNGVLNSWT |
| | | DQDSKDSTYSMSSTLTLTKDEYERHNSYTCEATHKTSTSPIV |
| | | KSFNRNEC |
| P1AD4006, | 92 | EVQLQESGPGLVKPSQSLSLTCSVTGYSITSSYRWNWIRKFP |
| <muPD1>RMP1- | (C01686648) | GNRLEWMGYINSAGISNYNPSLKRRISITRDTSKNQFFLQVN |
| 14muIgG2akappaPG LALA- | | SVTTEDAATYYCARSDNMGTTPFTYWGQGTLVTVSSASTTA |
| HC | | PSVYPLAPVCGDTTGSSVTLGCLVKGYFPEPVTLTWNSGSLS |
| | | SGVHTFPAVLQSDLYTLSSSVTVTSSTWPSQSITCNVAHPASS |
| | | TKVDKKIEPRGPTIKPCPPCKCPAPNAAGGPSVFIFPPKIKDVL |
| | | MISLSPIVTCVVVDVSEDDPDVQISWFVNNVEVHTAQTQTH |
| | | REDYNSTLRVVSALPIQHQDWMSGKEFKCKVNNKDLGAPIE |
| | | RTISKPKGSVRAPQVYVLPPPEEEMTKKQVTLTCMVTDFMP |
| | | EDIYVEWTNNGKTELNYKNTEPVLDSDGSYFMYSKLRVEK |
| | | KNWVERNSYSCSVVHEGLHNHHTTKSFSRTPGK |
| P1AG7552, muPD1-0103- | identical to | KIVLTQSPASLPVSLGQRATISCRASESVDTSDNSFIHWYQQR |
| mIgG1-DAPG-DD/KK- | #83 | PGQSPKLLIYRSSTLESGVPARFSGSGSRTDFTLTIDPVEADD |
| IL2v-LC | (C2184220339) | VATYYCQQNYDVPWTFGGGTKLEIKRADAAPTVSIFPPSSEQ |
| | | LTSGGASVVCFLNNFYPKDINVKWKIDGSERQNGVLNSWTD |
| | | QDSKDSTYSMSSTLTLTKDEYERHNSYTCEATHKTSTSPIVK |
| | | SFNRNEC |
| P1AG7552, muPD1-0103- | 93 | EVILVESGGGLVKPGGSLKLSCAASGFSFSSYTMSWVRQTPE |
| mIgG1-DAPG-DD/KK- | (C2184248915) | KRLDWVATISGGGRDIYYPDSVKGRFTISRDNAKNTLYLEM |
| IL2v-HC DD- | | SSLMSEDTALYYCVLLTGRVYFALDSWGQGTSVTVSSAKTT |
| | | PPSVYPLAPGSAAQTNSMVTLGCLVKGYFPEPVTVTWNSGS |
| | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGGGSGGGGSGGGG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLT |
| P1AG7552, muPD1-0103- | identical to | EVILVESGGGLVKPGGSLKLSCAASGFSFSSYTMSWVRQTPE |
| mIgG1-DAPG-DD/KK- | #85 | KRLDWVATISGGGRDIYYPDSVKGRFTISRDNAKNTLYLEM |
| IL2v-HC KK+ | (C2184220338) | SSLMSEDTALYYCVLLTGRVYFALDSWGQGTSVTVSSAKTT |
| | | PPSVYPLAPGSAAQTNSMVTLGCLVKGYFPEPVTVTWNSGS |
| | | LSSGVHTFPAVLQSDLYTLSSSVTVPSSTWPSQTVTCNVAHP |
| | | ASSTKVDKKIVPRDCGCKPCICTVPEVSSVFIFPPKPKDVLTIT |
| | | LTPKVTCVVVAISKDDPEVQFSWFVDDVEVHTAQTKPREEQ |
| | | INSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |
| P1AG9991, ratPD1- | identical to | DIVMTQGTLPNPVPSGESVSITCRSSKSLLYSDGKTYLNWYL |
| mIgG1-DAPG-DD/KK- | #87 | QRPGQSPQLLIYWMSTRASGVSDRFSGSGSGTDFTLKISGVE |
| IL2v non-masked-LC | (C01486562), | AEDVGIYYCQQGLEFPTFGGGTKLELKRADAAPTVSIFPPSSE |
| | = #10 | QLTSGGASVVCFLNNFYPKDINVKWKIDGSERQNGVLNSWT |
| | | DQDSKDSTYSMSSTLTLTKDEYERHNSYTCEATHKTSTSPIV |
| | | KSFNRNEC |
| P1AG9991, ratPD1- | 94 | EVQLQESGPGLVKPSQSLSLTCSVTGYSITSSYRWNWIRKFP |
| mIgG1-DAPG-DD/KK- | (C2184253826) | GNRLEWMGYINSAGISNYNPSLKRRISITRDTSKNQFFLQVN |
| IL2v non-masked-HC DD− | | SVTTEDAATYYCARSDNMGTTPFTYWGQGTLVTVSSAKTTP |
| | | PSVYPLAPGSAAQTNSMVTLGCLVKGYFPEPVTVTWNSGSL |
| | | SSGVHTFPAVLQSDLYTLSSSVTVPSSTWPSQTVTCNVAHPA |
| | | SSTKVDKKIVPRDCGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTISK |
| | | TKGRPKAPQVYTIPPPKEQMAKDKVSLTCMITNFFPEDITVE |
| | | WQWNGQPAENYDNTQPIMDTDGSYFVYSDLNVQKSNWEA |
| | | GNTFTCSVLHEGLHNHHTEKSLSHSPGGGGGSGGGGSGGGG |
| | | GAPASSSTKKTQLQLEHLLLDLQMILNGINNYKNPKLTRMLT |
| | | AKFAMPKKATELKHLQCLEEELKPLEEVLNGAQSKNFHLRP |
| | | RDLISNINVIVLELKGSETTFMCEYADETATIVEFLNRWITFA |
| | | QSIISTLT |
| P1AG9991, ratPD1- | identical to | EVQLQESGPGLVKPSQSLSLTCSVTGYSITSSYRWNWIRKFP |
| mIgG1-DAPG-DD/KK- | #89 | GNRLEWMGYINSAGISNYNPSLKRRISITRDTSKNQFFLQVN |
| IL2v non-masked-HC | (C2184220341) | SVTTEDAATYYCARSDNMGTTPFTYWGQGTLVTVSSAKTTP |
| KK+ | | PSVYPLAPGSAAQTNSMVTLGCLVKGYFPEPVTVTWNSGSL |
| | | SSGVHTFPAVLQSDLYTLSSSVTVPSSTWPSQTVTCNVAHPA |
| | | SSTKVDKKIVPRDCGCKPCICTVPEVSSVFIFPPKPKDVLTITL |
| | | TPKVTCVVVAISKDDPEVQFSWFVDDVEVHTAQTKPREEQI |
| | | NSTFRSVSELPIMHQDWLNGKEFKCRVNSAAFGAPIEKTISK |
| | | TKGRPKAPQVYTIPPPKKQMAKDKVSLTCMITNFFPEDITVE |
| | | WQWNGQPAENYKNTQPIMKTDGSYFVYSKLNVQKSNWEA |
| | | GNTFTCSVLHEGLHNHHTEKSLSHSPG |

### FURTHER ASPECTS OF THE DISCLOSURE

In a further aspect, the disclosure provides for a de-immunized PD-1 binder comprising a heavy chain variable region (VH) comprising a heavy chain complementary determining region (HCDR) 1 of SEQ ID NO: 71, a HCDR 2 of SEQ ID NO: 72, and a HCDR 3 of SEQ ID NO: 73, and a light chain variable region (VL) comprising a light chain complementarity determining region (LCDR) 1 of SEQ ID NO: 68, a LCDR 2 of SEQ ID NO: 69 and a LCDR 3 of SEQ ID NO: 70. In a specific aspect, the disclosure provides for a de-immunized PD1 binder comprising an amino acid sequence that is at least about 95%, 96%, 97%, 98%, 99% or 100% identical to the sequence of SEQ ID NO: 53 and an amino acid sequence that is at least about 95%, 96%, 97%, 98%, 99% or 100% identical to the sequence of SEQ ID NO: 54. In another aspect, the de-immunized PD-1 binder comprises an amino acid sequence according to SEQ ID NO: 53 and an amino acid sequence according to SEQ ID NO: 54. In another aspect, the disclosure provides for the use of the de-immunized PD-1 binder as disclosed herein, wherein the de-immunized PD-1 binder is present in a therapeutic agent. Preferably, the therapeutic agent is an isolated polypeptide. More preferably, the therapeutic agent is a cancer treatment.

In a further aspect, the disclosure provide for a de-immunized MT204-derived binder comprising a heavy chain variable region (VH) comprising a heavy chain complementary determining region (HCDR) 1 of SEQ ID NO: 77, a HCDR 2 of SEQ ID NO: 78, and a HCDR 3 of SEQ ID NO: 79, and a light chain variable region (VL) comprising a light chain complementarity determining region (LCDR) 1 of SEQ ID NO: 74, a LCDR 2 of SEQ ID NO: 75 and a LCDR 3 of SEQ ID NO: 76. In a specific aspect, the disclosure provides for a de-immunized MT204-derived binder comprising an amino acid sequence that is at least about 95%, 96%, 97%, 98%, 99% or 100% identical to the sequence of SEQ ID NO: 55 and an amino acid sequence that is at least about 95%, 96%, 97%, 98%, 99% or 100% identical to the sequence of SEQ ID NO: 56. In another aspect, the de-immunized MT204-derived binder comprises an amino acid sequence according to SEQ ID NO: 55 and an amino acid sequence according to SEQ ID NO: 56. In a further aspect, the de-immunized MT204-derived binder is a single-chain Fab. In a further aspect, the de-immunized MT204-derived binder is a single-chain Fv. In another aspect, the disclosure provides for the use of the de-immunized MT204-derived binder as disclosed herein, wherein the de-immunized MT204-derived binder is present in a therapeutic agent. Preferably, the therapeutic agent is an isolated polypeptide. More preferably, the therapeutic agent is a cancer treatment.

### EXAMPLES

The following are examples of methods and compositions of the invention. It is understood that various other embodiments may be practiced, given the general description provided above.

### Example 1

Design of Binders with Reduced Immunogenicity Potential

In order to reduce potential immunogenicity issues, we intended to increase the germline content of two binders binding to PD1 or interleukin 2 (IL2).

The PD1 binder (P1AA0927, SEQ ID NOs 51 and 52) was aligned to the human germline sequence IGHV3-23-01 (Acc No: M99660) for the heavy chain and IGKV4-1-01 (Acc No: Z00023) for the light chain, respectively. In addition we analyzed the protein sequences with a commercial software for the prediction of MHC class 2 binding peptides (Software: ISPRI; Provider: Epivax).

In particular, those sequence stretches with predicted MHC class 2 binding should be addressed, either by increasing the germline content, or by introducing mutations that reduce the MHC binding score. While maintaining affinity and stability of the antibody. Especially the sequence around the Framework 2 and CDR2 of the light chain showed three overlapping peptides with predicted MHC binding. Since germlining would reduce binding properties, we manually picked mutations that reduce the predicted binding score, and then we validated experimentally the maintenance of the desired biochemical properties as disclosed below.

For the heavy chain, we focused more on germlining. The leucine at Kabat position 5 was replaced by the more frequent valine at this position. Therefore, a higher number of human germline residues were introduced. The de-immunized PD1 binder used in the examples below comprises the VH sequence of SEQ ID NO:54 and the VL sequence of SEQ ID NO: 53.

The IL2 binder MT204 was processed in a similar way. The light chain was compared either to the human germline IGKV1-16-01, or to the trastuzumab VL (CAS number 180288-69-1) sequence, which is considered to be of low immunogenicity. For the heavy chain, also the sequence IGHV3-23-01 (Acc No: M99660) was used as the comparator. Optimized sequences then have a higher degree of human germline content. In addition, variant MT204_VL1a would have a higher homology to the trastuzumab framework (on Kabat position 54). The de-immunized MT204 used in the examples below comprises the VH sequence of SEQ ID NO:56 and the VL sequence of SEQ ID NO: 55.

### Production and Purification of De-Immunized MT204 Masks and De-Immunized Anti-PD1 Binder Variants as IgGs

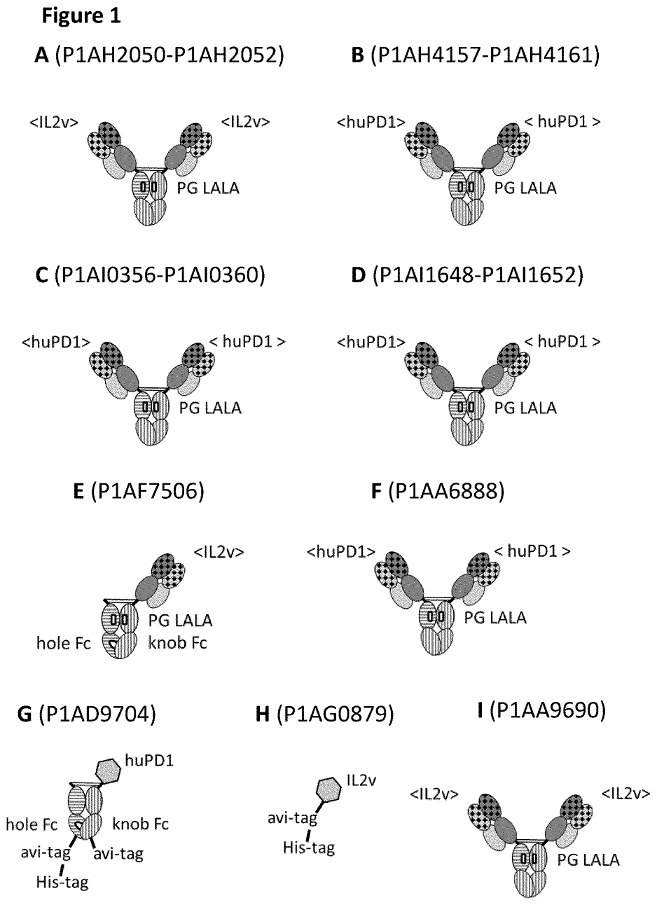

The de-immunized sequences of the anti-IL2v mask (P1AH2050-P1AH2052) and of the anti-PD1 binder (P1AH4157-P1AH4161, P1AI0356-P1AI0360, and P1AI1648-P1AI1652) were cloned as human IgG PG LALA for initial characterization ((FIG. 1A-1D). The corresponding cDNAs were gene synthesized and cloned into evitria's vector system using conventional (non-PCR based) cloning techniques. Plasmid DNA was prepared under low-endotoxin conditions based on anion exchange chromatography. DNA concentration was determined by measuring the absorption at a wavelength of 260 nm. Correctness of the sequences was verified with Sanger sequencing with two sequencing reactions per plasmid.

Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 μm filter).

The IgGs were purified using MabSelect™ SuRe™ with Dulbecco's PBS (Lonza BE17-512Q) as wash buffer, 0.1 M Glycine pH 3.5 as elution buffer and 1 M Tris HCl as neutralization buffer (pH 9). Subsequent size exclusion chromatography was performed on a HiLoad Superdex 200 pg column using the final buffer as running buffer. Dialysis (if needed) was performed using Pierce Slide-A-Lyzer™ G2 Dialysis Cassettes with a 2K molecular weight cut off. Antibody concentration (if needed) was performed using Amicon® Ultra Centrifugal Filters with a 30 kDa molecular weight cut off.

The concentration was determined by measuring absorption at a wavelength of 280 nm. The extinction coefficient was calculated using a proprietary algorithm at evitria. Purity was determined by analytical size exclusion chromatography with an Agilent AdvanceBio SEC column (300A 2.7 μm 7.8×300 mm) and DPBS as running buffer at 0.8 ml/min. Endotoxin content was measured with the Charles River Endosafe PTS system.

### Design, Production and Purification of Complex PD1-Targeted Masked IL2v Formats

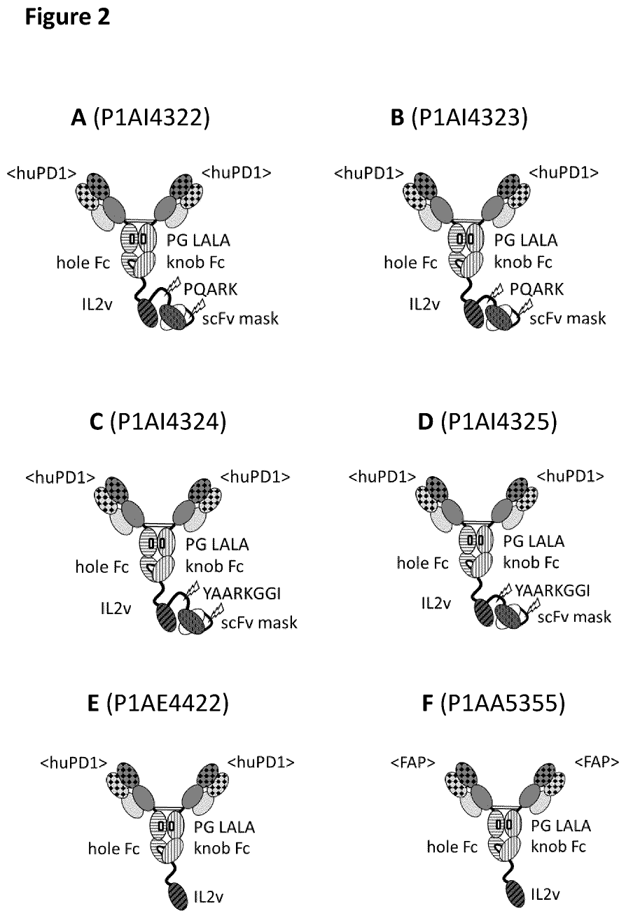

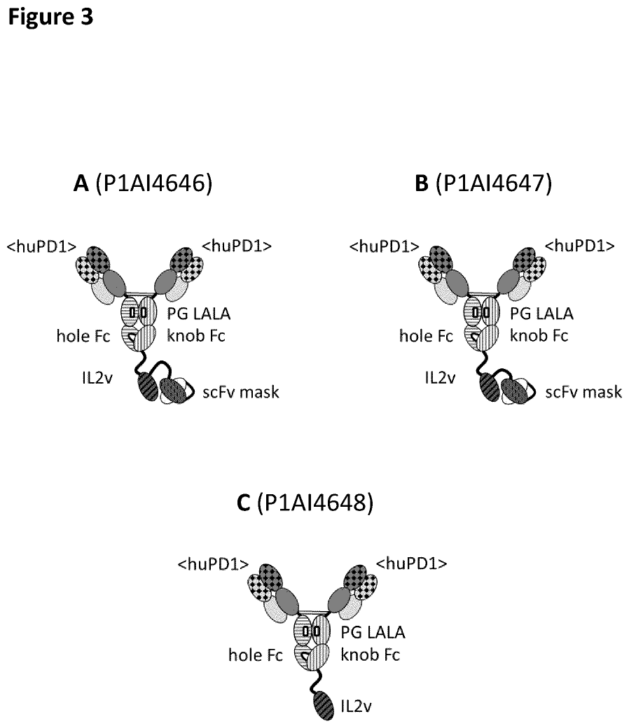

One de-immunized anti-IL2v mask sequence (derived from P1AH2051, i.e. hu IgG1 GL MT204_VL1a-combo VH2 & VH6 PG LALA) and one de-immunized anti-PD1 binder sequence (derived from P1AH4157, i.e. PD1-376_VL2_VH4) were used to clone 4 complex PD1-targeted masked IL2v formats (FIG. 2A-2D). They were cloned as bivalent human IgG1 PG LALA antibodies with knobs-into-holes heterodimerization of the two heavy chains. Masked IL2v, comprising the IL2v cytokine and the C-terminally fused scFv mask, was fused to the C-terminus of the knob heavy chain. Moreover, the glycine-serine linker sequences between the IL2v cytokine and the scFv mask as well as the glycine-serine linker between the VH and VL domains of the scFv mask, comprise a protease recognition site for specific unmasking and activation of IL2v by proteases in the tumor micro-environment, e.g. matriptase. The protease recognition site used in P1AI4322 and P1AI4323 was PQARK (FIG. 2A-2B), the protease recognition site used in P1AI4324 and P1AI4325 was YAARKGGI (FIG. 2C-2D). Moreover, the four constructs differ in the length of the protease recognition site comprising linker between the IL2v cytokine and the scFv mask, being 38 amino acids in P1AI4322 and P1AI4324 and 25 amino acids in P1AI4323 and P1AI4325. As control constructs, non-cleavable constructs P1AI4646 (with a 38 amino acid linker between the IL2v cytokine and the scFv mask) and P1AI4647 (with a 25 amino acid linker between the IL2v cytokine and the scFv mask) lacking the protease release sites as well as a non-masked construct P1AI4648 were generated (FIG. 3A-3C).

These complex PD1-targeted masked IL2v formats (P1AI4322, P1AI4323, P1AI4324 and P1AI4325) were produced and purified by WuXi Biologics. In brief, they were transiently transfected into HEK293 and purified by MabSelectSuRe LX protein A affinity chromatography and Superdex200 size-exclusion chromatography. Purification of P1AI4324 involved two additional HiTrap SP HP-1 and SP HP-2 cation exchange chromatography purification steps in between MabSelectSuRe LX protein A affinity chromatography and Superdex200 size-exclusion chromatography.

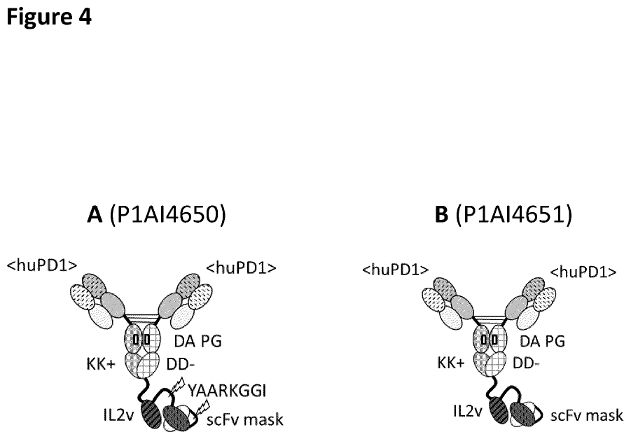

To facilitate in vivo efficacy studies in human PD1 transgenic mice, a cleavable (with YAARKGGI sites) and a non-cleavable human PD1-targeted masked IL2v construct was generated, P1AI4650 and P1AI4651, respectively (FIG. 4A-4B). These murine surrogates share the same format as the human constructs but comprise murine sequences to avoid immunogenicity. The V-domains of the PD1 binder correspond to the non-humanized predecessor whereas all constant antibody domain sequences are murine. The only human sequences in these surrogates that could not be avoided are the human IL2v as well as the V-domains of the mask. The murine surrogates were also produced and purified by WuXi Biologics.

### Surface Plasmon Resonance (Determination of Masking Affinities to Human IL2v)

Affinities of de-immunized MT204 variants (P1AH2050-P1AH2052, FIG. 1A) to human IL2v (SEQ ID NO:67) were assessed by surface plasmon resonance (SPR). The SPR experiment was performed on a Biacore 8K+ at 25 °C with HBS-EP running buffer (0.01 M HEPES pH 7.4, 0.15 M NaCl, 3 mM EDTA, 0.05% Surfactant P20, Cytiva, Freiburg/Germany).

Anti-PGLALA antibody (M-1.7.24 mulgG2b) was directly immobilized on a CM5 chip at pH 5.0 using the standard amine coupling kit (Cytiva, Freiburg/Germany). After activation of the sensor surface with a 1:1 mixture of 0.4 M 1-ethyl-3-(3-dimethylaminopropyl)-carbodiimide (EDC) and 0.1 M N-hydroxysuccinimide (NHS), 10 μg/ml anti-PGLALA (diluted in 10 mM acetate pH 5.0) was injected for 100 seconds with a flow rate of 10 μl/min. After blocking with 1 M ethanolamine-HCl pH 8.5, the coupling procedure led to approximately 5400 RU anti-PG LALA surface density.

De-immunized MT204 variants were captured for 60 s at a flow rate of 10 μl/min with a concentration of 50 nM. Human IL2v G4S avi his was injected at various concentrations (800-0.391 nM, 1:2 dilutions) with a flow of 30 μl/min through the flow cells. Association and dissociation were monitored for 240 s and 1000 s respectively. The chip surface was regenerated after every cycle by using one injection (60 s) of 10 mM glycine pH 2.0. Bulk refractive index differences were corrected by subtracting the response obtained on reference flow cell. Curves were fitted by using the 1:1 Langmuir interaction model by using Biacore Insight Evaluation Software 3.0 (Cytiva, Freiburg/Germany) and the results were summarized in Table 1.1.

TABLE 1.1

| construct ID | name | ka (1/Ms) | kd (1/s) | KD (M) | Rmax (RU) | Chi² (RU²) |
|---|---|---|---|---|---|---|
| P1AH2050 | hu IgG1 GL | 2.53E+05 | 1.08E−04 | 4.27E−10 | 52.0 | 4.08E−01 |
| | MT204_VL1 - combo | | | | | |
| | VH2 & VH6 PG LALA | | | | | |
| P1AH2051 | hu IgG1 GL | 2.35E+05 | 2.37E−05 | 1.01E−10 | 39.1 | 1.90E−01 |
| | MT204_VL1a - combo | | | | | |
| | VH2 & VH6 PG LALA | | | | | |
| P1AH2052 | hu IgG1 GL | 2.19E+05 | 2.18E−04 | 9.95E−10 | 39.1 | 1.84E−01 |
| | MT204_VL1b - combo | | | | | |
| | VH2 & VH6 PG LALA | | | | | |
| P1AF7506 | OA MT204 | 2.68E+05 | 7.24E−05 | 2.70E−10 | 37.2 | 4.74E−01 |
| | IgG PG LALA | | | | | |

Two of the three de-immunized variants of the anti-IL2v mask, P1AH2050 and P1AH2051, were comparable to the parental anti-IL2v mask P1AF7506 (FIG. 1E) in terms of kinetic rate constants and affinities, whereas the third de-immunized variant, P1AH2052, exhibited the fastest off-rate of all of the tested variants and with 995 μM also the lowest affinity. Variant P1AH2051, i.e. hu IgG1 GL MT204_VL1a-combo VH2 & VH6 PG LALA, exhibiting the highest affinity of 101 pM, was selected for cloning of the complex masked IL2v formats.

### Surface Plasmon Resonance for the Determination of Affinities to Human PD1

Affinities of de-immunized a-PD1 binders to human PD1-Fc were assessed by surface plasmon resonance (SPR). The SPR experiments were performed either on a Biacore T200 (for P1AH4157-P1AH4161 and P1AI0356-P1AI0360) or on a Biacore 8K+ (for P1AI1648-P1AI1652). P1AH4157-P1AH4161 and P1AI0356-P1AI0360 (FIG. 1B-C)) were characterized on a Biacore T200 at 25 °C with HBS-EP running and sample dilution buffer (0.01 M HEPES pH 7.4, 0.15 M NaCl, 3 mM EDTA, 0.05% Surfactant P20, Cytiva, Freiburg/Germany). Anti-PGLALA antibody (M-1.7.24 mulgG2b) was directly immobilized on a CM5 chip at pH 5.0 using the standard amine coupling kit (Cytiva, Freiburg/Germany). After activation of the sensor surface with a 1:1 mixture of 0.4 M 1-ethyl-3-(3-dimethylaminopropyl)-carbodiimide (EDC) and 0.1 M N-hydroxysuccinimide (NHS), 20 μg/ml anti-PGLALA (diluted in 10 mM acetate pH 5.0) was injected for 900 s with a flow rate of 5 μl/min. After blocking with 1 M ethanolamine-HCl pH 8.5, the coupling procedure led to more than 12000 RU anti-PG LALA surface density.

De-immunized a-PD1 binders were captured for 60 s at a flow rate of 10 μl/min with a concentration of 10 nM. Recombinant huPD1-ECD_Fc-knob/hole (internal ID P1AD9704) was injected at various concentrations (300-9.4 nM, 1:1 dilution) with a flow of 50 μl/min through the flow cells. Association and dissociation were monitored for 120 s and 600 s respectively. The chip surface was regenerated after every cycle by using injection (35 s each) of 20 mM NaOH. Bulk refractive index differences were corrected by subtracting the response obtained on reference flow cell. Curves were fitted by using the 1:1 Langmuir interaction model by using Biacore T200 Evaluation Software 3.1 (GE Healthcare Bio-Sciences) and the results were summarized in the tables below.

P1AI1648-P1AI1652 (FIG. 1D) were characterized on a Biacore 8K+ at 25 °C with HBS-EP running buffer (0.01 M HEPES pH 7.4, 0.15 M NaCl, 3 mM EDTA, 0.05% Surfactant P20, Cytiva, Freiburg/Germany).

Anti-PGLALA antibody (M-1.7.24 mulgG2b) was directly immobilized on a C1 chip at pH 5.0 using the standard amine coupling kit (Cytiva, Freiburg/Germany). After activation of the sensor surface with a 1:1 mixture of 0.4 M 1-ethyl-3-(3-dimethylaminopropyl)-carbodiimide (EDC) and 0.1 M N-hydroxysuccinimide (NHS), 20 μg/ml anti-PGLALA (diluted in 10 mM acetate pH 5.0) was injected for 10 min with a flow rate of 10 μl/min. After blocking with 1 M ethanolamine-HCl pH 8.5, the coupling procedure led to approximately 1000 RU anti-PG LALA surface density.

De-immunized a-PD1 binders were captured for 80 s at a flow rate of 10 μl/min with a concentration of 5 nM. Recombinant huPD1-ECD_Fc-knob/hole was injected at various concentrations (200-0.27 nM, 1:3 dilution) with a flow of 30 μl/min through the flow cells. Association and dissociation were monitored for 240 s and 800 s respectively. The chip surface was regenerated after every cycle by using two injections (60 s each) of 10 mM glycine pH 2. Bulk refractive index differences were corrected by subtracting the response obtained on reference flow cell. Curves were fitted by using the 1:1 Langmuir interaction model by using Biacore Insight Evaluation Software 3.0 (Cytiva, Freiburg/Germany) and the results were summarized in Tables 1.2, 1.3 and 1.4.

TABLE 1.2
Binding properties

| construct ID | name | ka (1/Ms) | kd (1/s) | KD (nM) | t ½ (min) | Rmax (%) |
|---|---|---|---|---|---|---|
| P1AH4157 | PD1-376_VL2_VH4 | 6.40E+05 | 1.30E−04 | 0.2 | 86 | 106 |
| P1AH4158 | PD1-376_VL7_VH4 | 1.90E+05 | 2.80E−04 | 1.5 | 41 | 83 |
| P1AH4159 | PD1-376_VL2_7_VH4 | 6.30E+05 | 1.40E−04 | 0.2 | 80 | 106 |
| P1AH4160 | PD1-376_VL7_VH1_2_4 | 2.10E+05 | 2.50E−04 | 1.2 | 47 | 83 |
| P1AH4161 | PD1-376_VL2_VH1_2_3_4 | 5.00E+05 | 2.10E−04 | 0.4 | 54 | 105 |
| P1AA6888 | one-shot humanized PD1-0103 | 6.30E+05 | 1.50E−04 | 0.2 | 79 | 129 |
| | (FIG. 1F) | | | | | |

TABLE 1.3
Binding properties

| construct ID | name | ka (1/Ms) | kd (1/s) | KD (nM) | t ½ (min) | Rmax (%) |
|---|---|---|---|---|---|---|
| P1AI0356 | PD1-1030-VH_I58L_D62E | 4.3E+05 | 1.0E−04 | 0.2 | 111 | 100 |
| P1AI0357 | PD1-1030-VH_I58T | 3.7E+05 | 8.9E−05 | 0.2 | 129 | 96 |
| P1AI0358 | PD1-1030-VH_I58T_P61A | 4.7E+05 | 1.3E−04 | 0.3 | 90 | 101 |
| P1AI0359 | PD1-1030-VH_P61A | 4.6E+05 | 1.0E−04 | 0.2 | 111 | 105 |
| P1AI0360 | PD1-1030-VH_Y60F_D62E | 4.0E+05 | 1.0E−04 | 0.3 | 113 | 103 |
| P1AA6888 | one-shot humanized PD1-0103 | 4.2E+05 | 1.1E−04 | 0.3 | 108 | 102 |
| | (FIG. 1F) | | | | | |

TABLE 1.4
Binding properties

| construct ID | name | ka (1/Ms) | kd (1/s) | KD (M) | Rmax (RU) | Chi² (RU²) |
|---|---|---|---|---|---|---|
| P1AG9606-008 | PD1 IgG-IL2v aIL2 | 8.03E+05 | 1.52E−04 | 1.90E−10 | 55.7 | 0.265 |
| | MT204 scFv - PQARK | | | | | |
| P1AH4157-001 | PD1-376_VL2_VH4 | 1.03E+06 | 1.30E−04 | 1.26E−10 | 72.6 | 0.557 |
| | IgG PG LALA | | | | | |
| P1AI1648-001 | PD1-376_VL2_VH4_I58L_D62E | 1.00E+06 | 1.45E−04 | 1.44E−10 | 66.1 | 0.592 |
| | IgG PG LALA | | | | | |
| P1AI1649-001 | PD1-376_VL2_VH4_I58T | 1.18E+06 | 1.30E−04 | 1.11E−10 | 65 | 0.569 |
| | IgG PG LALA | | | | | |
| P1AI1650-001 | PD1-376_VL2_VH4_I58T_P61A | 1.08E+06 | 1.36E−04 | 1.27E−10 | 74.2 | 0.592 |
| | IgG PG LALA | | | | | |
| P1AI1651-001 | PD1-376_VL2_VH4_P61A | 1.19E+06 | 1.35E−04 | 1.14E−10 | 74.3 | 0.844 |
| | IgG PG LALA | | | | | |
| P1AI1652-001 | PD1-376_VL2_VH4_Y60F_D62E | 8.50E+05 | 1.38E−04 | 1.62E−10 | 74.1 | 2.110 |
| | IgG PG LALA | | | | | |

In total, three sets of five de-immunized anti-PD1 variants each have been generated for assessment as human IgG1 PG LALA antibodies: Set 1 (P1AH4157-P1AH4161; FIG. 1B), set 2 (P1AI0356-P1AI0360; FIG. 1C), and set 3 (P1AI1648-P1AI1652; FIG. 1D). Sets 1 and 2 were independent sets whereas set 3 was based on P1AH4157 (PD1-376_VL2_VH4) with additional mutations in CDR H2. The kinetics rate constants and affinity of PD1-376_VL2_VH4 to human PD1 were very similar to the parental humanized antibody P1AA6888 (PD1-0103) with ka 6.40E+05, kd 1.30E-04, and KD 200 pM vs. ka 6.30E+05, kd 1.50E-04, and KD 200 pM, respectively. Based on binding properties and cell-based in vitro assessment, P1AH4157 (PD1-376_VL2_VH4) was chosen as preferred de-immunized anti-PD1 binder and its V-domain sequences were used for cloning of the complex masked IL2v formats.

### Example 2

Relative Luminescence Units of PD1/PD-L1 Reporter Jurkat Cell Line Upon PD-1/PD-L1 Blockade with Either Parental (Bivalent Versus Monovalent) or De-Immunized Anti-PD1 Antibodies

The PD-1/PD-L1 Blockade reporter assay from Promega (Cat. at. #J1250, J1255), is a bioluminescent cell-based assay that can be used to measure the potency of antibodies designed to block the PD-1/PD-L1 interaction. The assay consists in the co-culture of two genetically engineered cell lines: PD-1 T cells as effectors and PD-L1 APC/CHO-K1 cells as target cells. PD-1 effector T cells are Jurkat T cells expressing a human PD-1 and a luciferase reporter gene driven by NFAT response element (NFAT-RE). PD-L1 APC/CHO-K1 target cells are CHO-K1 cells expressing human PD-L1 and an engineered cell surface protein designed to activate cognate TCRs in an antigen-independent manner. When co-cultured, the PD-1/PD-L1 interaction inhibits TCR signaling and downregulates NFAT-RE-mediated luminescence in the effector T cells. The blockade of the PD-1/PD-L1 interaction by anti-PD-1 (or anti-PD-L1) antibodies releases the inhibitory signal and results in TCR activation of NFAT-RE mediated luminescence. This assay was used to assess the ability of the de-immunized PD-1 binders to block the PD-1/PD-L1 interaction. For this purpose, eight 1:10 dilution steps with 66 nM as the highest concentration of either PD1 IgG PG LALA PD1-IL2v or de-immunized anti-PD-1 were added to the PD-L1-CHO-expressing cells, right before co-culturing the two cell lines for 5 hours at 37 °C. After the 5 hours of incubation, the substrate (BIO-Glo Reagent) was added and the samples were measured at the luminometer (PerkinElmer Reader).

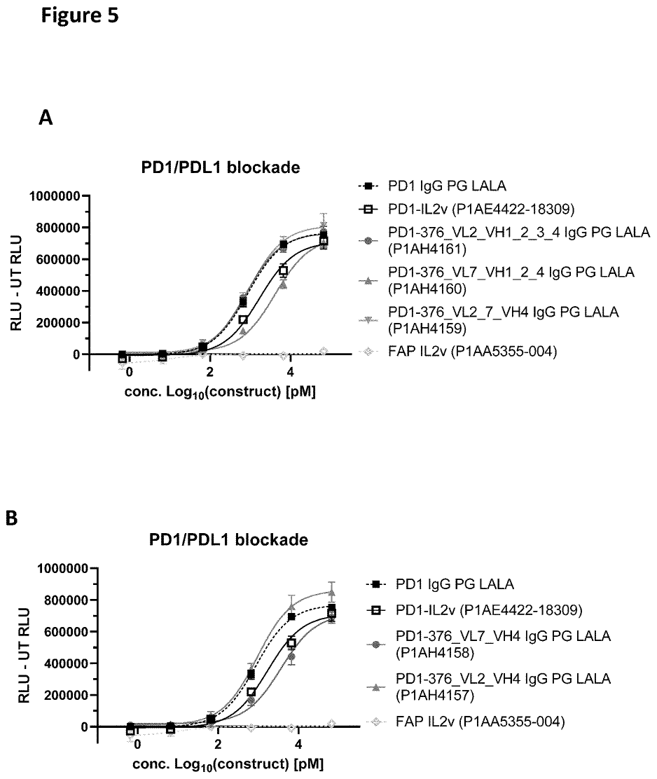

FIGS. 5A and 5B show that PD1-376_VL2_VH1_2_3_4 IgG PG LALA, PD1-376_VL2_7_VH4 IgG PG LALA and PD1-376_VL2_VH4 IgG PG LALA were approximately as potent as the parental anti-PD1 in blocking the PD-1/PD-L1 interaction and overcoming the downstream inhibitory signals. On the other hand, mutations on PD1-376_VL7_VH1_2_4 IgG PG LALA and PD1-376_VL7_VH4 IgG PG LALA result in a decreased potency of 4-5 fold (Table 2.1).

TABLE 2.1
EC50 of blocking potency of the dose-response as Relative Luminescence
Units for the de-immunized PD-1 on the PD-1 Effector cells.

| Molecule | EC50 |
|---|---|
| PD-1 IgG PF LALA | 862.3 |
| PD1-IL2v (P1AE4422-18309) | 1671 |
| PD1-376_VL2_VH1_2_3_4 IgG PG LALA | 767.5 |
| PD1-376_VL7_VH1_2_4 IgG PG LALA | 4058 |
| PD1-376_VL2_7_VH4 IgG PG LALA | 911.7 |
| PD1-376_VL7_VH4 IgG PG LALA | 3620 |
| PD1-376_VL2_VH4 IgG PG LALA | 922.4 |
| FAP IL2v (P1AA5355-004) | 9.71 |

PD1-IL2v (as disclosed in WO 2018/184964 A1) and FAP IL2v (as disclosed in WO 2012/107417 A1; INN: Simlukafusp alfa) were used as controls and they showed that the lack of aPD-1 in FAP-IL2v prevents the PD-1/PD-L1 inhibition of TCR signaling and the resulting luminescence. On the other hand as the interaction is blocked by the aPD-1 on the PD1-IL2v construct, NFAT-RE-mediated luminescence is active.

### Binding of De-Immunized PD-1 to Activated CD4 T Cells

In the following experiment, de-immunized PD-1 antibodies were compared side by side in a binding assay in order to assess whether the de-immunization affects the binding affinity/avidity for PD-1 on T cells. For this purpose, CD4 T cells were isolated from healthy donor PBMCs with CD4 beads (#130-045-101, Miltenyi) and activated for 3 days in presence of 1 μg/ml plate bound anti-CD3 (overnight pre-coated, clone OKT3, #317326, BioLegend) and 1 μg/ml of soluble anti-CD28 (clone CD28.2, #302934, BioLegend) antibodies to induce T cell activation and PD-1 expression. Three days later, the cells were harvested and washed to remove endogenous IL-2. Cells were then seeded into a V-bottom plate before being stained for 30 min at 4 °C with increasing concentrations of treatment antibodies: PD1-376_VL2_VH4 IgG PG LALA, PD1-376_VL7_VH4 IgG PG LALA, PD1-376_VL2_7_VH4 IgG PG LALA, PD1-376_VL7_VH1_2_4 IgG PG LALA, PD1-376_VL2_VH1_2_3_4 IgG PG LALA, parental anti-PD-1 PD1-IL2v and FAP IL2v (50 μl, 1:10 dilution steps with the highest concentration of 66 nM).

The cells were washed with PBS to remove unbound molecules. Then, 50 μl of the diluted AF647 anti-PGLALA antibody and Fixable Viability Dye eFluor™ 780 (eBioscience) were added to the cells followed by a washing step after 30 min of incubation at 4 °C. Finally, the cells were fixed with BD cell fix (70 ul, #340181, BD Biosciences) and acquired at the FACSymphony A3 Cell Analyzer (BD Bioscience). The frequency and MFI of positive cells were determined with FlowJo (V10) and plotted with GraphPad Prism.

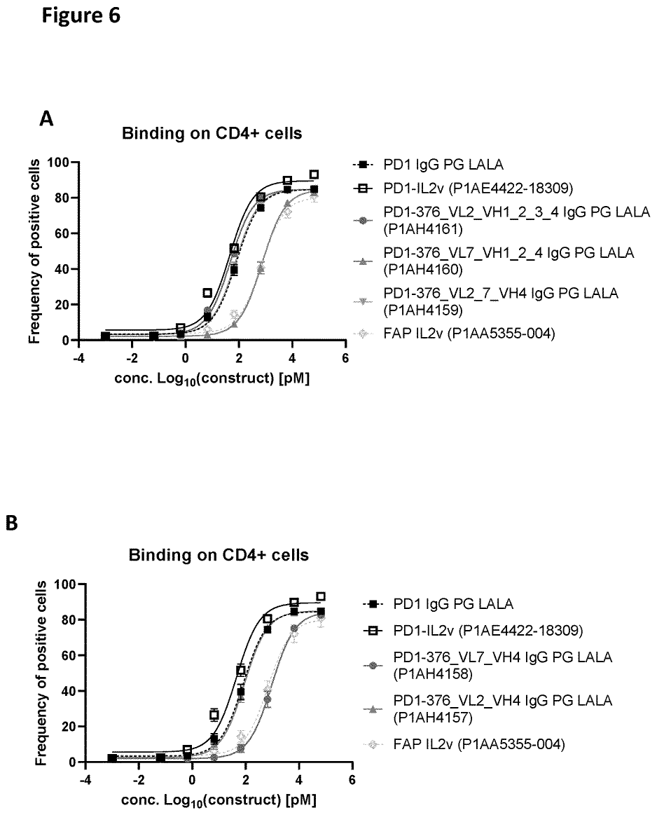

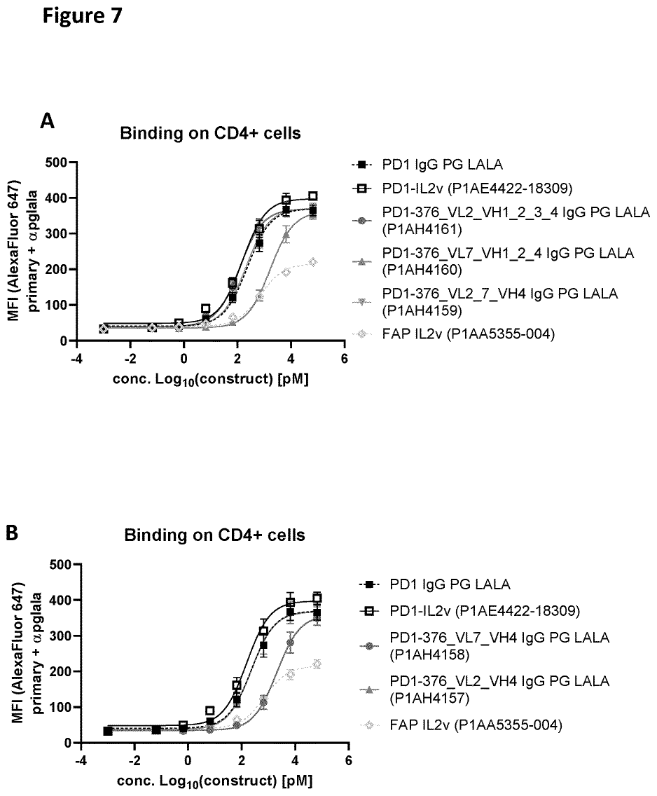

As shown in FIGS. 6A and 6B and 7A and 7B, PD1-376_VL2_VH4 IgG PG LALA, PD1-376_VL2_7_VH4 IgG PG LALA, PD1-376_VL2_VH1_2_3_4 IgG PG LALA and the corresponding parental anti-PD1- and PD1-IL2v bind with similar potency to CD4 T cells. PD1-376_VL7_VH4 IgG PG LALA and PD1-376_VL7_VH1_2_4 IgG PG LALA show decreased binding to CD4 T cells. FAP-IL2v served as an untargeted control to compare to the effect of IL2v based immunocytokine alone that are not targeted to PD-1.

Tables 2.2 and 2.3 show the EC50 of the frequency and MFI of the dose-response binding of the various antibodies to activated CD4 T cells obtained from 2 donors.

TABLE 2.2
EC50 of binding of the dose-response frequency for the selected
de-immunized PD-1 on PD-1⁺ CD4 T cells from healthy donors.

| Molecule | EC50 |
|---|---|
| PD-1 IgG PF LALA | 80.62 |
| PD1-IL2v (P1AE4422-18309) | 45.49 |
| PD1-376_VL2_VH1_2_3_4 IgG PG LALA | 47.92 |
| PD1-376_VL7_VH1_2_4 IgG PG LALA | 747.6 |
| PD1-376_VL2_7_VH4 IgG PG LALA | 74.42 |
| PD1-376_VL7_VH4 IgG PG LALA | 948.4 |
| PD1-376_VL2_VH4 IgG PG LALA | 88.64.4 |
| FAP IL2v (P1AA5355-004) | 9.679.8 |

TABLE 2.3
EC50 of binding of the dose-response MFI for the selected
de-immunized PD-1 on PD-1⁺ CD4 T cells from healthy donors.

| Molecule | EC50 |
|---|---|
| PD-1 IgG PF LALA | 234.2 |
| PD1-IL2v (P1AE4422-18309) | 153.6 |
| PD1-376_VL2_VH1_2_3_4 IgG PG LALA | 121.1 |
| PD1-376_VL7_VH1_2_4 IgG PG LALA | 1655 |
| PD1-376_VL2_7_VH4 IgG PG LALA | 206 |
| PD1-376_VL7_VH4 IgG PG LALA | 2052 |
| PD1-376_VL2_VH4 IgG PG LALA | 226.5 |
| FAP IL2v (P1AA5355-004) | 708 |

Effect of Different Anti-PD-1 Antibodies on Cytotoxic Granzyme B and IFN-γ Secretion by Allospecific Human CD4 T Cells Co-Cultured with Allogeneic Mature Dendritic Cells

In order to assess the de-immunized PD-1 antibody in a functional assay, CD4 T cells were exposed to allogenic matured DCs to induce the generation of allospecific T cells harboring different antigen-specificities and TCR-affinities. Importantly, allospecific T cells express immune checkpoints like PD-1 and therefore are useful to measure the functional potency of anti-PD1 antibodies as they can unleash T cell effector functions. To screen for the functionality and potency of PD-1 blocking antibodies in an allogeneic setting, freshly purified CD4 T cells were co-cultured for 5 days in presence of monocyte-derived allogeneic mature dendritic cells (mDCs). Monocytes were isolated from fresh PBMCs one week before with CD14 beads (130-050-201, Miltenyi). Immature DCs were generated from the monocytes by culturing them for 5 days in media containing GM-CSF (50 ng/ml) and IL-4 (100 ng/ml). To induce iDCs maturation, TNF-α, IL-1β and IL-6 (50 ng/ml each) were added to the culturing media for 2 additional days. On the day of the minimal mixed lymphocyte reaction (mMLR), CD4 T cells were enriched via a microbead kit (Miltenyi) from PBMCs obtained from an unrelated donor. Prior to culture, CD4 T cells were labeled with 5 μM of Cell Trace Violet (CTV, #C34557, ThermoFisher). 105 CD4 T cells were then plated in flat-bottom 96-well plates together with mature allogeneic-DCs (10:1 ratio) for 5 days at 37 °C, 5% CO₂, in presence of a concentration range of purified anti-PD1 monoclonal antibodies: PD1-376_VL2_VH4 IgG PG LALA, PD1-376_VL7_VH4 IgG PG LALA, PD1-376_VL2_7_VH4 IgG PG LALA, PD1-376_VL7_VH1_2_4 IgG PG LALA, PD1-376_VL2_VH1_2_3_4 IgG PG LALA, parental anti-PD1 and PD1-IL2v (50 μl, 1:10 dilution steps with the highest concentration of 66 nM). Either no antibody or FAP IL2v was used as a negative control.

After five days, the cells were left at 37 °C for additional 5 hours in presence of Golgi Plug (Brefeldin A) and Golgi Stop (Monensin), before being washed, stained on the surface with anti-human CD4 antibody and Fixable Viability Dye eFluor™ 780 (eBioscience) and fixed/permeabilized with Fix/Perm Buffer (BD Bioscience). The cells were later stained intracellularly for Granzyme B (BD Bioscience) and IFN-γ (eBioscience). Results are shown in FIG. G/H and I/J (GrzB and IFN-γ secretion/release).

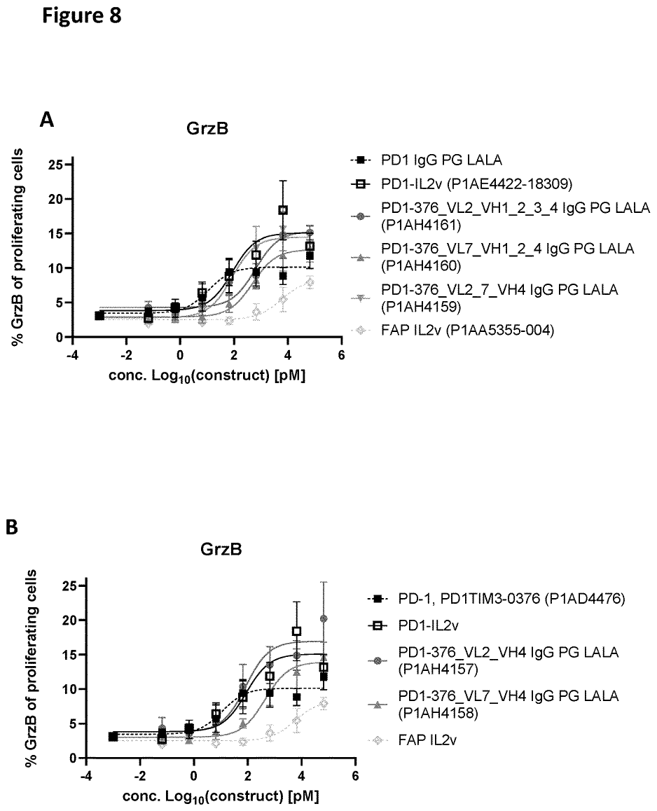

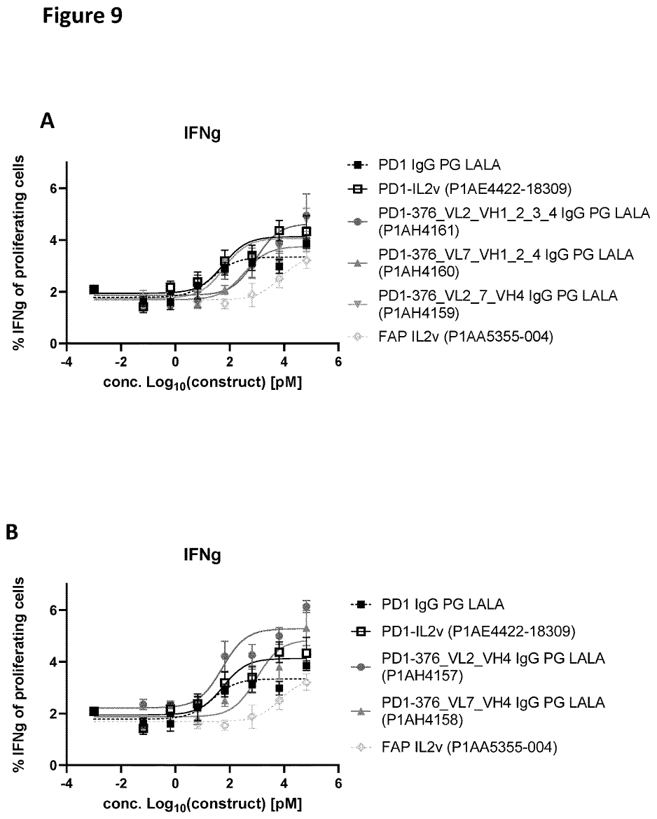

The anti-PD1 monoclonal antibodies promoted T cell secretion of GrzB (FIGS. 8A and 8B) and IFN-γ (FIGS. 9A and 9B) in a concentration dependent manner. All anti-PD-1 variants were found to enhance granzyme B and IFN γ in comparison to FAP-IL2v or untreated cells (negative controls). Tables 2.4 and 2.5 show the EC50 and Area Under the Curve (AUC) of the % of GrzB and IFNγ secretion of the dose-dependent response of CD4 T cells to allogenic stimulation and treatment with anti-PD-1 antibody.

TABLE 2.4
EC50 and Area Under the Curve of the dose-response Granzyme B secretion
for the selected de-immunized PD-1 on CD4 T cells from healthy donors.

| | % GrzB | Total | Total | | Peak Y − |
|---|---|---|---|---|---|
| Molecule | (EC50) | area* | peak* | Peak Y | Baseline |
| PD-1 IgG PF LALA | 11.33 | 27.22 | 27.22 | 11.85 | 8.757 |
| PD1-IL2v (P1AE4422-18309) | 87.84 | 39.56 | 39.17 | 18.38 | 15.28 |
| PD1-376_VL2_VH1_2_3_4 IgG PG LALA | 624.1 | 31.91 | 31.91 | 15.13 | 12.03 |
| PD1-376_VL7_VH1_2_4 IgG PG LALA | 494.4 | 21.72 | 20.37 | 12.85 | 9.757 |
| PD1-376_VL2_7_VH4 IgG PG LALA | 84.71 | 33.79 | 32.87 | 15.61 | 12.51 |
| PD1-376_VL7_VH4 IgG PG LALA | 440.1 | 25.33 | 24.04 | 14.6 | 11.51 |
| PD1-376_VL2_VH4 IgG PG LALA | 84.02 | 42.33 | 42.33 | 20.21 | 17.12 |
| FAP IL2v (P1AA5355-004) | 5591 | 8.567 | 5.215 | 7.938 | 4.847 |

TABLE 2.5
EC50 and Area Under the Curve of the dose-response IFN-γ secretion
for the selected de-immunized PD-1 on CD4 T cells from healthy donors.

| | % IFNg | Total | Total | | Peak Y − |
|---|---|---|---|---|---|
| Molecule | (EC50) | area* | peak* | Peak Y | Baseline |
| PD-1 IgG PF LALA | 23.66 | 4.811 | 3.741 | 3.85 | 1.765 |
| PD1-IL2v (P1AE4422-18309) | 57.19 | 7.058 | 6.168 | 4.368 | 2.283 |
| PD1-376_VL2_VH1_2_3_4 IgG PG LALA | 898.7 | 6.271 | 4.732 | 4.943 | 2.858 |
| PD1-376_VL7_VH1_2_4 IgG PG LALA | 338 | 5.203 | 3.029 | 4.04 | 1.895 |
| PD1-376_VL2_7_VH4 IgG PG LALA | 60.8 | 6.464 | 5.255 | 4.783 | 2.698 |
| PD1-376_VL7_VH4 IgG PG LALA | 969 | 6.256 | 4.826 | 5.305 | 3.22 |
| PD1-376_VL2_VH4 IgG PG LALA | 52.57 | 10.17 | 10.17 | 6.135 | 4.05 |
| FAP IL2v (P1AA5355-004) | 6958 | 3.264 | 0.9177 | 3.218 | 1.133 |

The lower EC50 of cytokine production was obtained with the parental anti-PD-1, followed by PD1-376_VL2_VH4 IgG PG LALA. The highest AUC was provided by the de-immunized variant PD1-376_VL2_VH4 IgG PG LALA and the control PD1-IL2v.

### Example 3

### Binding of De-Immunized PD1 Binder to CHO-huPD1 Cells

We assessed the binding of five different de-immunized PD1-IgGs to human PD1 overexpressing CHO cells in comparison to the respective parental PD1-IgG molecule and TA PD1-IL2v construct containing the parental PD1 binder (Table 3.1).

TABLE 3.1
Tested constructs

| Constructs | ID |
|---|---|
| PD1-376_VL2_VH4 IgG PG LALA | P1AH4157-001 |
| PD1-376_VL7_VH4 PG LALA | P1AH4158-001 |
| PD1-376_VL2_7_VH4 PG LALA | P1AH4159-001 |
| PD1-376_VL7_VH1_2_4 PG LALA | P1AH4160-001 |
| PD1-376_VL2_VH1_2_3_4 PG LALA | P1AH4161-001 |
| PD1 IgG-IL2v aIL2 MT204 scFv - PQARK (parental) | P1AG9606-008 |
| PD1-IgG | P1AD4476-002 |

CHO-huPD1 cells (CHO-K1_MUSMU_PDCD1_Clone_42) were harvested with Trypsin/EDTA, washed with PBS and resuspended in FACS buffer (PBS, 2% FBS, 5 mM EDTA, 0.025% NaN3). Then, 100′000 cells were seeded per well in a round bottom plate. The cells were stained with 30 μl of de-immunized PD1-IgGs, the parental PD1-IgG and the TA PD1-IL2v construct containing the parental PD1-IgG at the indicated concentrations in FACS buffer for 30 min at 4 °C. After the staining, the cells were washed twice with FACS buffer to remove unbound molecules. Then, 30 μl of the diluted PE anti-human Fc specific secondary antibody (1:50 dilution, 109-116-170, Jackson ImmunoResearch) was added to the cells. After 30 min incubation at 4 °C the cells were washed twice with FACS buffer. Finally, the cells were resuspended in 150 μl FACS buffer and measured using a BD Fortessa.

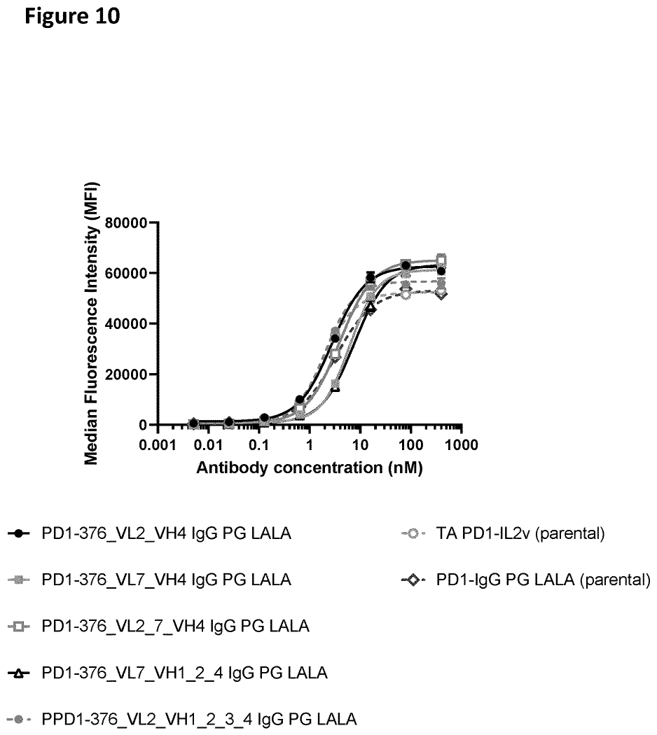

All five de-immunized PD1-IgGs bound similarly to human PD1 on CHO cells compared to the respective parental PD1-IgG, the 2 VL7 containing PD1-IgGs had a slightly reduced binding compared to the other de-immunized PD1-IgGs (FIG. 10).

Blocking IL2v Activity with De-Immunized MT204 Mask

Next, we tested the blocking of FAP-IL2v activity by three different de-immunized MT204 masks compared to the parental MT204 mask (Table 3.2).

TABLE 3.2
Tested constructs

| Constructs | ID |
|---|---|
| hu IgG1 GL MT204_VL1 - | P1AH2050-001 |
| combo VH2 & VH6 PG LALA | |
| hu IgG1 GL MT204_VL1a - | P1AH2051-001 |
| combo VH2 & VH6 PG LALA | |
| hu IgG1 GL MT204_VL1b - | P1AH2052-002 |
| combo VH2 & VH6 PG LALA | |
| anti-IL2 MM7 MM8 PG LALA, | P1AA9690-001 |
| anti MT204 | |
| FAP-IL2v | P1AA5355-004 |

NK92 cells were harvested, counted and assessed for viability. Cells were washed three times with PBS to remove residual IL2. The washed NK92 cells were re-suspended in fresh medium (advanced RPMI1640, 2% FCS, 1% Glutamine) without IL2 and 50 μl of the cell suspension containing 10,000 cells were transferred in a 96-well cell culture treated flat bottom plate. First, 25 μl of 0.5 nM FAP-IL2v antibody was added per well. Then, 25 μl of the MT204 antibodies was added per well to reach a final volume of 100 μl per well. The plate was incubated for 3 days in the incubator. After 3 days, the CellTiter-Glo (G7571, Promega) reagents and the cell culture plate were equilibrated to room temperature. The CellTiter-Glo solution was prepared as described in the manufacturer's instructions and 100 μl of the solution were added to each well. After 10 min of incubation, remaining aggregates were re-suspended by pipetting and 100 μl of the mixture were transferred to a 96 well white flat bottom plate. The luminescence was measured with a Tecan Spark 10M multimode reader.

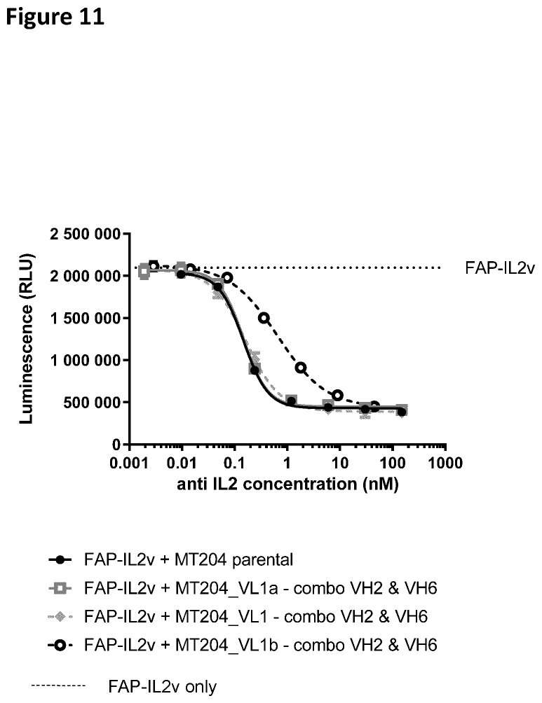

MT204 VL1 and MT204_VL1a blocked the IL2v activity comparable to the parental MT204 mask. The MT204_VL1b showed less efficient blocking of the IL2v activity compared to the parental MT204 mask (FIG. 11).

### Binding of TA PD1-IL2v Constructs to CHO-Hu PD1 Cells

Next, four different TA PD1-IL2v constructs were tested comparing a PQARK cleavage site, a YAARKGGI cleavage site, a 38 mer linker and a 25 mer linker.

TABLE 3.3
Tested constructs

| Tested constructs | ID |
|---|---|
| pro-PD1-IL2v_C-term_masked_PQARK_38mer | P1AI4322-004 |
| pro-PD1-IL2v_C-term_masked_PQARK_25mer | P1AI4323-004 |
| pro-PD1-IL2v_C-term_masked_YAARKGGI_38mer | P1AI4324-004 |
| pro-PD1-IL2v_C-term_masked_YAARKGGI_25mer | P1AI4325-004 |
| pro-PD1-IL2v_C-term_masked_non-cleavable_38mer | P1AI4646-001 |
| pro-PD1-IL2v_C-term_masked_non-cleavable_25mer | P1AI4647-001 |
| pro-PD1-IL2v_C-term_non-masked | P1AI4648-001 |
| clinical lead PD1-IL2v | P1AG5741-007 |
| pro-PD1-IL2v_C-term_masked_PQARK_38mer, | P1AG9606-008 |
| parental non-de-immunized PD1 binder and | |
| parental non-de-immunized MT204 mask | |

CHO-huPD1 cells (CHO-K1_MUSMU_PDCD1_Clone_42) were harvested with Trypsin/EDTA, washed with PBS and resuspended in FACS buffer (PBS, 2% FBS, 5 mM EDTA, 0.025% NaN3). 100,000 cells were seeded per well in a round bottom plate. The cells were stained with 30 μl of the TA PD1-IL2v constructs in FACS buffer for 30 min at 4 °C. After the staining, the cells were washed twice with FACS buffer to remove unbound molecules. Then 30 μl of the diluted APC anti-human Fc specific secondary antibody (1:50 dilution, 109-136-098, Jackson ImmunoResearch) was added to the cells. After 30 min incubation at 4 °C the cells were washed twice with FACS buffer. Finally the cells were resuspended in 150 μl FACS buffer and measured using a BD Fortessa.

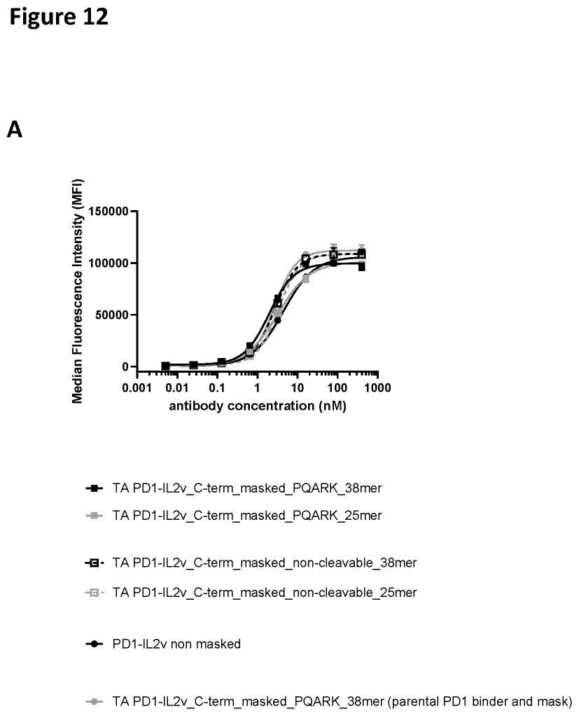

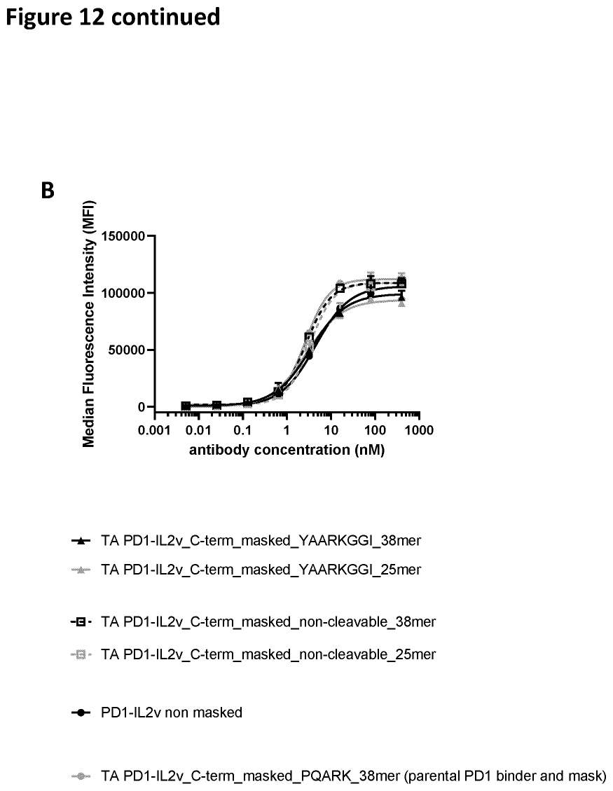

All four tested constructs contain the de-immunized VL2_VH4 PD1 binder and the MT204 VL1a de-immunized mask. We tested their binding to human PD1 overexpressing CHO cells in comparison to the respective non-cleavable, the unmasked and the parental TA PD1-IL2v molecule. All tested TA PD1-IL2v constructs bind comparable to human PD1 on CHO cells (FIG. 12).

### KHYG-1 Proliferation Induced by TA PD1-IL2v Constructs

Next, we tested the induction of proliferation of KHYG-1 cells by four different TA PD1-IL2v constructs, containing a PQARK cleavage site or a YAARKGGI cleavage site and a 38 mer linker or a 25 mer linker (see Table 3.3).

The NK cell line KHYG-1 was used to test proliferation induction by the TA PD1-IL2v constructs. Cells were harvested, counted and assessed for viability. Cells were washed three times with PBS to remove residual IL2. The washed KHYG-1 cells were re-suspended in fresh medium (advanced RPMI1640, 2% FCS, 1% Glutamine) without IL2 and 12.5 μl of the cell suspension containing 2′000 cells was transferred in a 384-well cell culture treated flat bottom plate. 10 μg of the TA PD1-IL2v constructs were digested with 2 μl Matriptase (Enzo~2.5 U/μl, ALX-201-246-U25, Lot 08102104 or without Matriptase as undigested control) for 2 hours at 37 °C in 20 μl Matriptase buffer (50 mM Tris, 50 mM NaCl, 0.01% Tween 20, pH 9.0) and 12.5 μl of the antibodies were added per well to reach a final volume of 25 μl per well. The plate was incubated for 3 days in the incubator. After 3 days, the CellTiter-Glo (G7571, Promega) reagents and the cell culture plate were equilibrated to room temperature. The CellTiter-Glo solution was prepared as described in the manufacturer's instructions and 25 μl of the solution were added to each well. After 10 min of incubation, remaining aggregates were re-suspended by pipetting and 40 μl of the mixture were transferred to a white flat bottom plate. The luminescence was measured with a Tecan Spark 10M multimode reader.

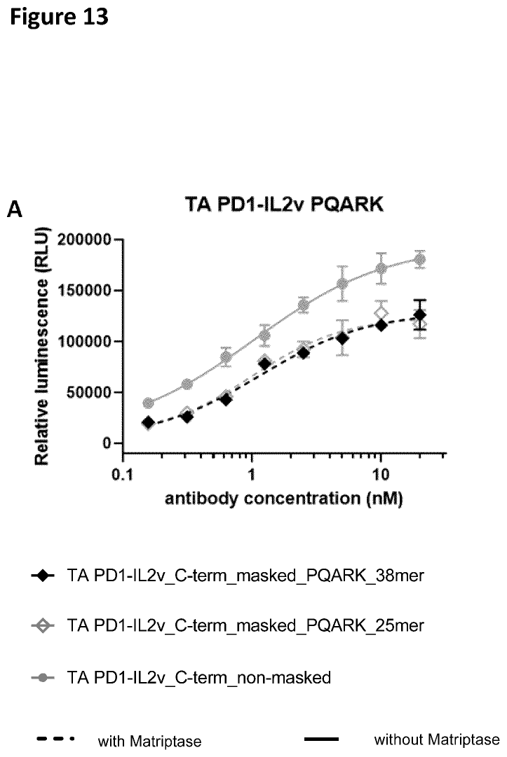

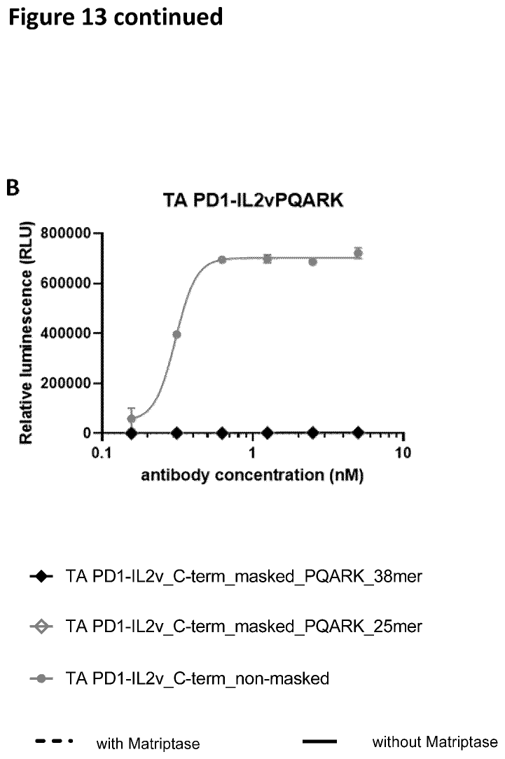

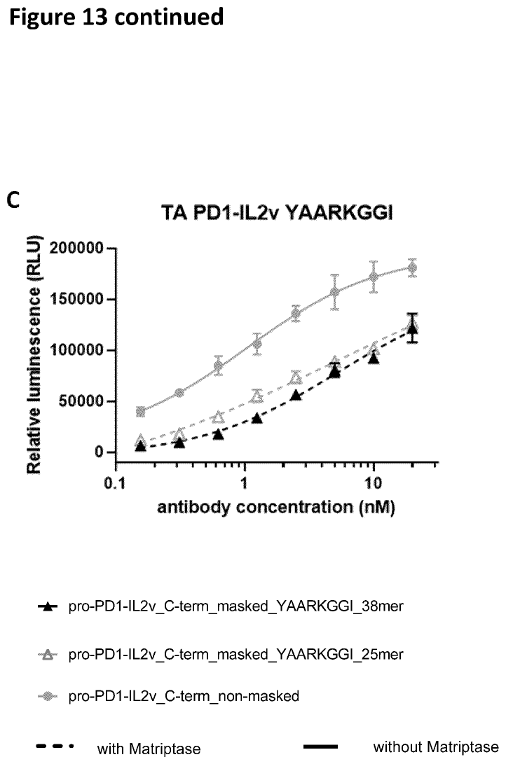

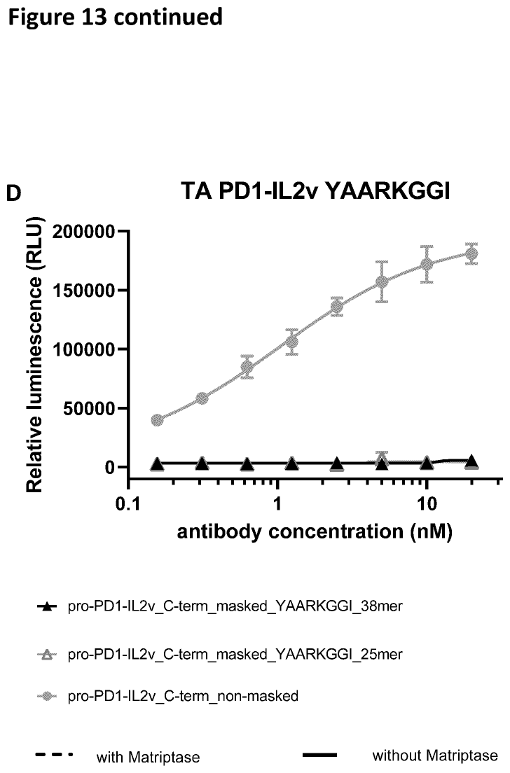

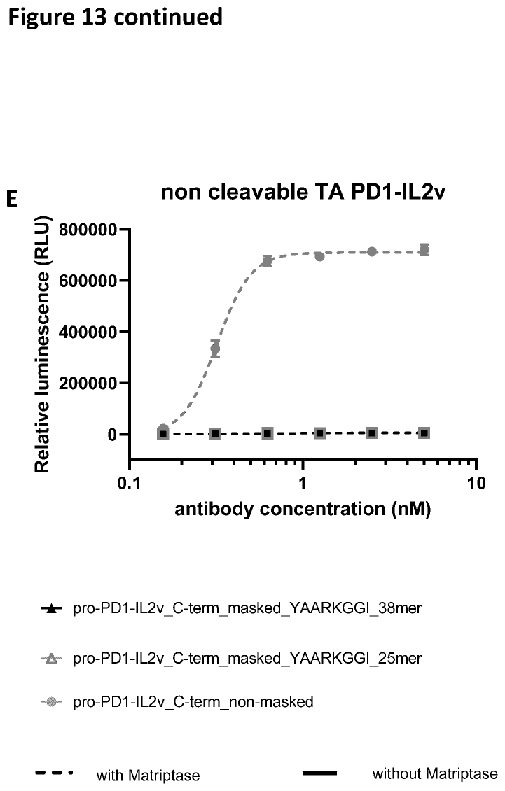

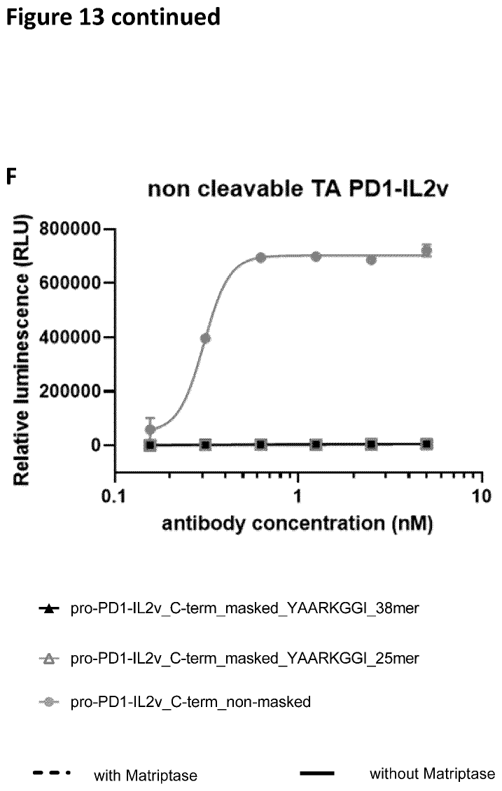

All four tested constructs contained the de-immunized VL2 VH4 PD1 binder and the MT204 VL1a de-immunized mask. As controls, the two respective non-cleavable molecules and the non-masked PD1-IL2v were included. All molecules were tested undigested and upon digestion with recombinant matriptase. After the digestion with matriptase, the TA PD1-IL2v with the PQARK and the YAARKGGI linker regained activity. The activity is slightly reduced compared to the unmasked control most probably due to incomplete cleavage. The activity of the TA PD-IL2v construct containing the 38 mer linker compared to the 25 mer linker are comparable (FIG. 13A, 13C). No undigested TA PD1-IL2v induced proliferation (FIG. 13A, 13D, 13F). The non-cleavable TA PD1-IL2v digested with matriptase did not induce proliferation (FIG. 13E). The unmasked PD1-IL2v activity is not affected by cleavage with matriptase compared to the non-cleavable construct (FIG. 13E, 13F).

### Example 4

### In Vivo Efficacy of Murinized TA-PD1-IL2v Immuno-Conjugates in a Syngeneic Model of Mouse Tumor Cell Line (MCA205 Subcutaneous Syngeneic Model)

The murinized TA-PD1-IL2v immuno-conjugates was tested in the mouse fibrosarcoma cell line MCA205, injected subcutaneously into Black 6-huPD1 transgenic mice.

The MCA205 fibrosarcoma carcinoma cells were originally obtained from Sigma Aldrich (Catalogue Number SCC173) and after expansion deposited in the Roche-Glycart internal cell bank. The tumor cell line was routinely cultured in DMEM containing 10% FCS (Gibco) at 37 °C in a water-saturated atmosphere at 5% CO₂. Passage 11 was used for transplantation, at a viability of 95.3%, 1×10⁶ cells per animal were injected subcutaneously in 100 μl of RPMI cell culture medium (Gibco) into the flank of mice using a 1 ml tuberculin syringe (BD Biosciences, Germany).

Female Black 6-huPD1 mice, aged 10-11 weeks at the start of the experiment (bred at Charles Rivers, Lyon, France) were maintained under specific-pathogen-free condition with daily cycles of 12 h light/12 h darkness according to committed guidelines (GV-Solas; Felasa; TierschG). The experimental study protocol was reviewed and approved by local government (P 184/2020). After arrival, animals were maintained for one week to get accustomed to the new environment and for observation. Continuous health monitoring was carried out on a regular basis.

Mice were injected subcutaneously on study day 0 with 1×10⁶ of MCA205 cells, randomized and weighed. Ten days after the tumor cell injection (tumor volume>150 mm³), mice were injected i.v. with TA-PD1-IL-2v YAARKGGI 38 mer cleavable linker or TA-PD1-IL-2v 38 mer non cleavable linker twice a week for one week. All mice were injected i.v. with 200 μl of the appropriate solution. The mice in the Vehicle group were injected with Histidine Buffer. To obtain the proper amount of immunoconjugate per 200 μl, the stock solutions were diluted with Histidine Buffer when necessary (Table 4).

TABLE 4

| | Dose/ | Concentration |
|---|---|---|
| Compound | mouse | (mg/mL) |
| TA-PD1-IL2v YAARKGGI | 40 μg | 2.10 |
| cleavable 38mer, | | (=stock solution) |
| P1AI4650 | | |
| TA-PD1-IL2v non- | 40 μg | 2.70 |
| cleavable 38mer, | | (=stock solution) |
| P1AI4651 | | |

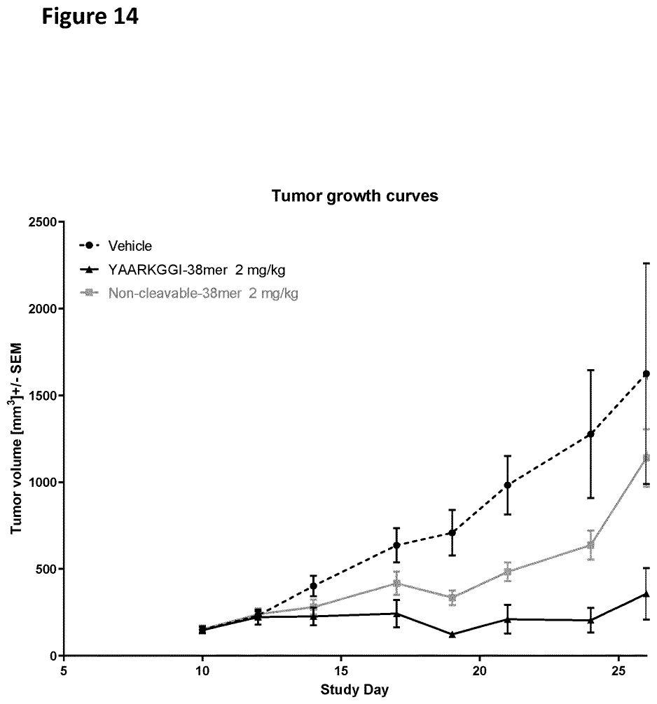

FIG. 14 shows that the TA-PD-IL2v YAARKGGI 38 mer mediated superior efficacy in terms of tumor growth inhibition compared to vehicle and non-cleavable Mab single agent groups.

### Example 5

Design of Murine Surrogates of Complex PD1-Targeted Masked IL2v Immunoconjugates with a scFv Mask

In order to facilitate in vivo tolerability and efficacy studies in non-tumor bearing mice or mouse models of cancer, murine surrogates of PD1-targeted masked IL2v immunoconjugates, that target either human (P1AK3638 and P1AK3649) or murine (P1AK3641 and P1AK3640) PD1, were generated. In order to decrease immunogenicity, all constant antibody domains in these constructs correspond to murine sequences. The murine surrogates are either targeted to human PD1 for use in humanized mice or human PD1 transgenic mice or murine PD1 for use in syngeneic mouse models with immunocompetent mice. Due to the cross-reactivity of human IL2v to murine IL2 receptors and the lack of cross-reactivity of the scFv mask to murine IL2v, human IL2v has been used in all construct.

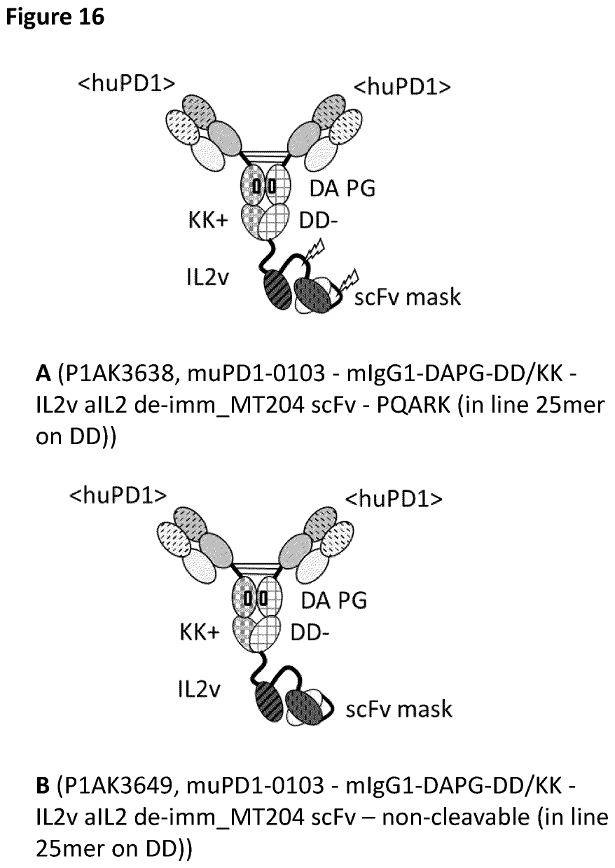

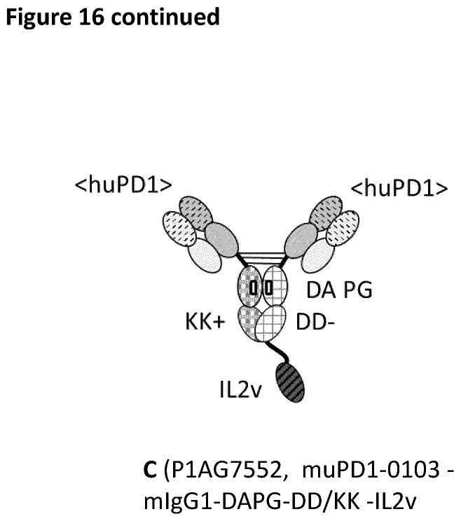

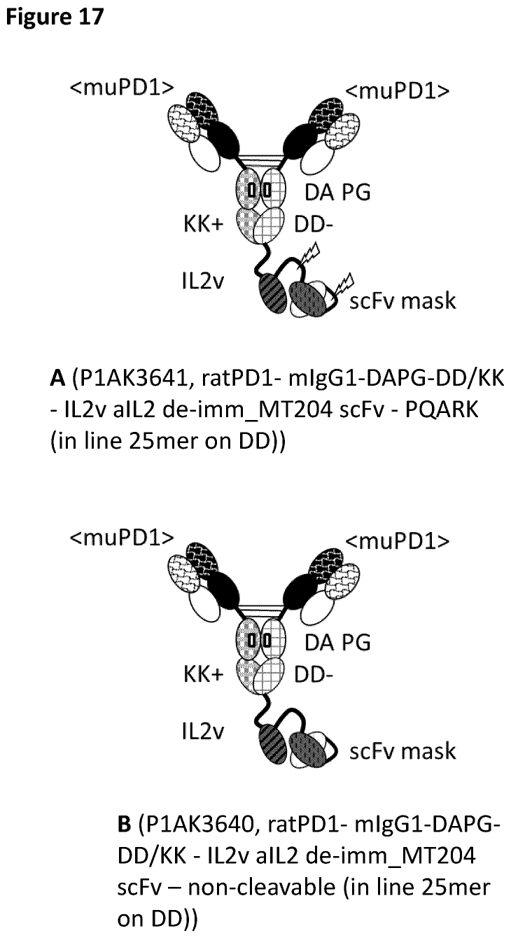

These murine surrogate constructs bind bivalently to either human or murine PD1 via the N-terminal Fab arms on the Fc DD− and Fc KK+ chains whereas the Fc DD− chain additionally carries a masked (matriptase cleavable or non-matriptase cleavable) C-terminal IL2v. The scFv mask is C-terminally fused ‘in-line’ on the same Fc DD-chain as IL2v. Heterodimerization was achieved by application of complementary charges in the murine IgG1 CH3 domains (Fc DD- and Fc KK+ chains) and binding to activating Fcγ receptors as well as complement component C1q has been abrogated by introduction of DA PG mutations in the murine IgG1 CH2 Fc domains of the antibodies. The matriptase-cleavable C-terminally masked IL2v constructs, P1AK3638 and P1AK3641, respectively, carry two PQARK matriptase recognition sites, wherein one PQARK matriptase recognition site is positioned in the linker between the VH and VL domains of the scFv mask and another one in the linker between the scFv mask and the IL2v. Additionally, respective non-matriptase cleavable control constructs (devoid of PQARK matriptase recognition sites), P1AK3649 and P1AK3640, respectively, and non-masked control constructs (devoid of the scFv mask) have been generated. These masked constructs are schematically depicted in FIGS. 16A-C and 17A-B, D. As a comparator, in order to test in vivo efficacy of checkpoint inhibition only, a murine IgG2a with anti-murine PD1 specificity (P1AD4006) was included in some of the studies and is depicted in FIG. 17C.

### Production and Purification of PD1-Targeted Masked IL2v Immunoconjugates and Control Constructs

The murine surrogate PD1-targeted masked IL2v immunoconjugates (P1AK3638 and P1AK3649 as well as P1AK3641 and P1AK3640, respectively) have been produced and purified by WuXi Biologics. They were transiently expressed in HEK293 and purified in a 2-column DSP process: 1. MabSelectSuRe LX affinity chromatography (equilibration and 1st wash: 25 mM Tris-HCl, 150 mM NaCl, 5 mM EDTA, pH 7.5; 2nd wash: 25 mM Tris-HCl, 150 mM NaCl, 5 mM EDTA, 0.1% Triton 100/114, pH 7.5; elution: 50 mM Sodium Citrate-Citric, 150 mM NaCl, pH 3.0; neutralization: 1 M Arginine, 0.4 M Succininc Acid, pH 9.0); and 2. Superdex200 size-exclusion chromatography (equilibration and formulation buffer: 20 mM Histidine-HCl, 140 mM NaCl, pH 6.0). Purity has been determined by SEC-HPLC as well as reduced and non-reduced Caliper-SDS. Purification batches were tested for low endotoxin levels and the identity of the de-glycosylated masses has been confirmed by liquid chromatography-mass spectrometry (LC-MS).

The murine IgG2a with anti-murine PD1 specificity, P1AD4006, was produced and purified at evitria AG. Suspension-adapted CHO K1 cells (originally received from ATCC and adapted to serum-free growth in suspension culture at evitria) were used for production. The seed was grown in eviGrow medium, a chemically defined, animal-component free, serum-free medium. Cells were transfected with eviFect, evitria's custom-made, proprietary transfection reagent, and cells were grown after transfection in eviMake2, an animal-component free, serum-free medium. Supernatant was harvested by centrifugation and subsequent filtration (0.2 μm filter). The IgG was purified using MabSelect™ SuRe™ with Dulbecco's PBS (Lonza BE17-512Q) as wash buffer, 0.1 M Glycine pH 3.5 as elution buffer and 1 M Tris HCl as neutralisation buffer (pH 9). Subsequent size exclusion chromatography was performed on a HiLoad Superdex 200 μg column using the final buffer as running buffer. Dialysis (if needed) was performed using Pierce Slide-A-Lyzer™ G2 Dialysis Cassettes with a 2K molecular weight cut off. Antibody concentration (if needed) was performed using Amicon® Ultra Centrifugal Filters with a 30 kDa molecular weight cut off. The concentration was determined by measuring absorption at a wavelength of 280 nm. The extinction coefficient was calculated using a proprietary algorithm at evitria. Purity was determined by analytical size exclusion chromatography with an Agilent AdvanceBio SEC column (300A 2.7 μm 7.8×300 mm) and DPBS as running buffer at 0.8 ml/min. Endotoxin content was measured with the Charles River Endosafe PTS system.

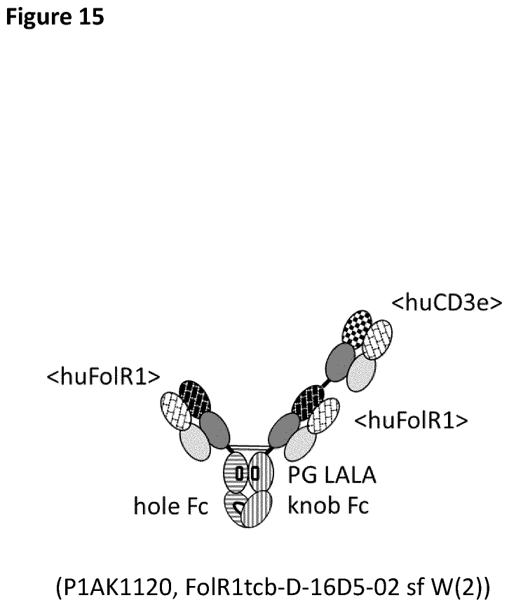

The human FolR1-targeted T cell engager, P1AK1120 (FIG. 15), that was used to provide T cell ‘signal l’ in some of the in vivo efficacy studies, has been produced by evitria AG as described above and purified at Roche in a 3-column DSP process (1. Protein A MabSelectSure, 2. Butyl HP Hydrophobic Interaction Chromatography (HIC), and 3. Superdex200 HiLoad 16/6000 size-exclusion chromatography (SEC)) and formulated into 20 mM Histidine, 140 mM NaCl, pH 6.0. Purity has been determined by SEC-HPLC as well as reduced and non-reduced CE-SDS. The purification batch was tested for a low endotoxin level and the identity of the de-glycosylated masses has been confirmed by liquid chromatography-mass spectrometry (LC-MS).

### Example 6

HEK Blue IL2 Reporter Cell Assay with HEK Blue IL2 Cells Overexpressing Human PD1 to Test Activity of Murinized TA PD1-IL2v Constructs

We tested the activity of murinized cleavable and non-cleavable TA PD1-IL2v molecules containing a human specific PD1 binder in the HEK blue IL2 reporter cell assay using HEK blue IL2 cell expressing human PD1.

The calculated amount of each molecule was digested with 2 μl recombinant human Matriptase (ALX-201-246-U250, Enzo) and filled up to 20 μl with Matriptase buffer (50 mM Tris, 50 mM NaCl, 0.01% Tween 20, pH 9.0). The corresponding amount of undigested controls was also filled up to 20 μl with Matriptase buffer and treated subsequently in the same way as samples where Matriptase was added. The digest was done for 2 hours at 37 °C. After digestion, the samples were filled up with DMEM+10% FBS+1% GlutaMax (assay medium) to have a final starting concentration of 50 nM.

HEK-Blue huPD1 IL-2 cells (HEK-Blue-IL2_hPDCD1_clone 4) were detached using Cell Dissociation Buffer and re-suspended in DMEM+10% FBS+1% GlutaMax (assay medium) at 0.33 Mio cells/ml. 150 μl of HEK-Blue IL-2 cells (containing 50′000 cells) were then seeded in a 96 well flat bottom plate. Then 50 μl of titrated surrogate IL2v molecules were added to each well to reach a final volume of 200 μl per well. The plate was then incubated for 24 hours at 37 °C. Then 180 μl of Quanti-Blue solution (rep-qbs2, InvivoGen) and 20 μl of cell supernatant were added per well in a 96 well flat bottom plate and incubated for 60 min at 37 °C. Afterwards, the Optical density (OD) was measured at 620 nm with a Tecan Spark Reader.

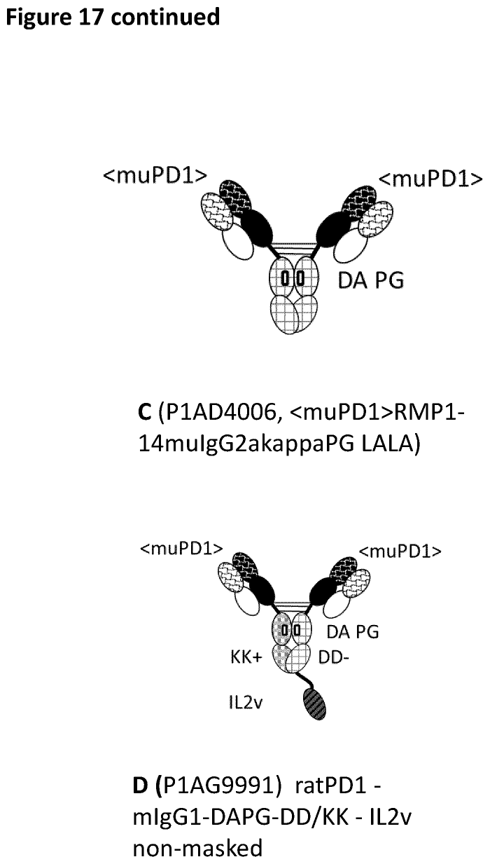

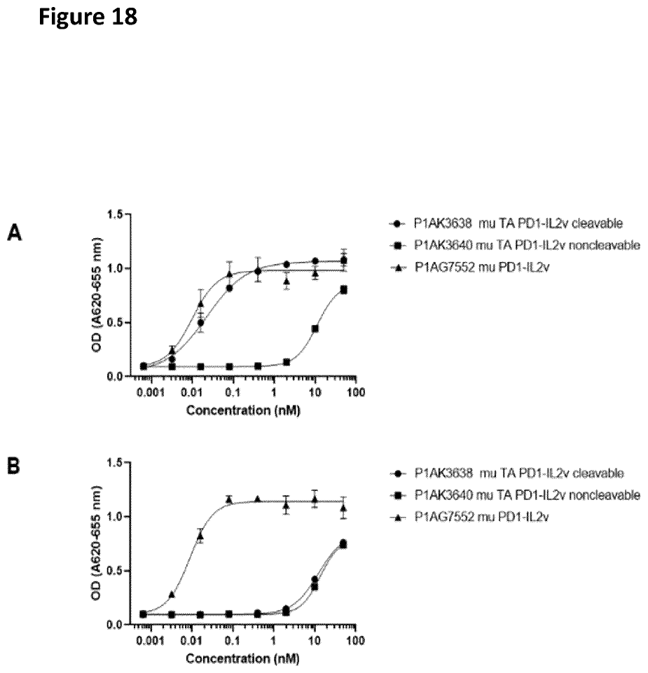

Activity of the molecules was compared to the respective unmasked PD1-IL2v construct. All molecules were tested either after cleavage with recombinant Matriptase or undigested. Upon cleavage, the activity of the cleavable murinized TA PD1-IL2v construct is comparable to naked PD1-IL2v whereas the non-cleavable TA PD1-IL2v has only very limited activity in the reporter cell assay (FIG. 18A).

In the absence of digestion with recombinant Matriptase, the cleavable and the non-cleavable TA PD1-IL2v constructs have both only very limited activity compared to naked PD1-IL2v (FIG. 18B).

### Example 7

HEK Blue IL2 Reporter Cell Assay with HEK Blue IL2 Cells Overexpressing Mouse PD1 to Test Activity of Murinized TA PD1-IL2v Constructs Containing a Mouse Specific PD1 Binder.

We tested the activity of murinized cleavable and non-cleavable TA PD1-IL2v molecules containing a mouse specific PD1 binder in the HEK blue IL2 reporter cell assay using HEK blue IL2 cells expressing mouse PD1.

The calculated amount of each molecule was digested with 2 μl recombinant human Matriptase (ALX-201-246-U250, Enzo) and filled up to 20 μl with Matriptase buffer (50 mM Tris, 50 mM NaCl, 0.01% Tween 20, pH 9.0). The corresponding amount of undigested controls was also filled up to 20 μl with Matriptase buffer and treated subsequently in the same way as samples where Matriptase was added. The digest was done for 2 hours at 37 °C. After digestion, the samples were filled up with DMEM+10% FBS+1% GlutaMax (assay medium) to have a final starting concentration of 50 nM.

HEK-Blue huPD1 IL-2 cells (HEK-Blue IL-2 cells_muPDCD1_clone 12 cells) were detached using Cell Dissociation Buffer and re-suspended in DMEM+10% FBS+1% GlutaMax (assay medium) at 0.33 Mio cells/ml. 150 μl of HEK-Blue IL-2 cells (containing 50′000 cells) were then seeded in a 96 well flat bottom plate. Then 50 μl of titrated surrogate IL2v molecules were added to each well to reach a final volume of 200 μl per well. The plate was then incubated for 24 hours at 37 °C. Then 180 μl of Quanti-Blue solution (rep-qbs2, InvivoGen) and 20 μl of cell supernatant were added per well in a 96 well flat bottom plate and incubated for 60 min at 37 °C. Afterwards, the Optical density (OD) was measured at 620 nm with a Tecan Spark Reader.

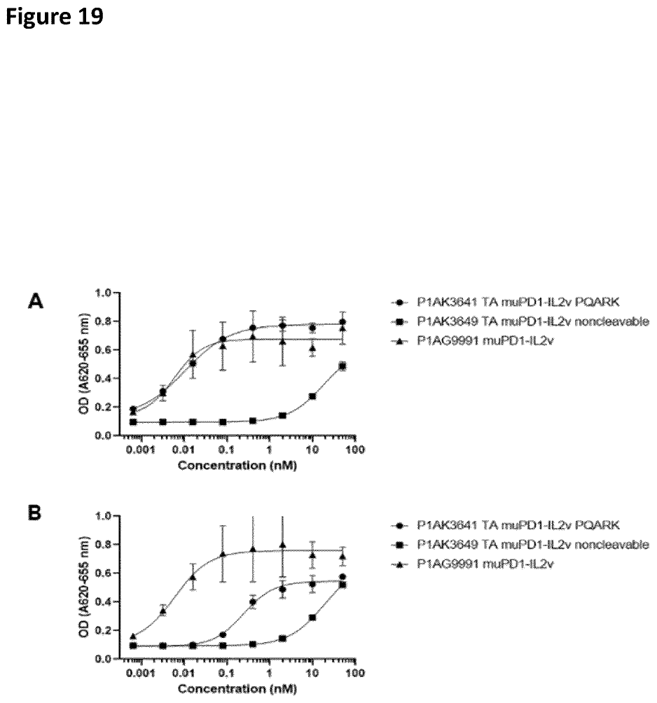

Activity of the molecules was compared to the respective unmasked PD1-IL2v construct. All molecules were tested either after cleavage with recombinant Matriptase or undigested. Upon cleavage, the activity of the cleavable murinized TA PD1-IL2v construct is comparable to naked PD1-IL2v whereas the non-cleavable TA PD1-IL2v has only very limited activity in the reporter cell assay (FIG. 19A).

In the absence of predigestion with recombinant Matriptase, the cleavable and the non-cleavable TA PD1-IL2v constructs have both only very limited activity compared to naked PD1-IL2v. The TA PD1-IL2v cleavable constructs have slightly higher activity than the respective non-cleavable constructs that might indicate minor cleavage that can happen during the incubation time (FIG. 19B).

### Example 8

### In Vitro Proliferation and Activation on CD8 T Cells and NK Cells Induced by TA PD1-IL2v Constructs.

TA PD1-IL2v constructs were tested for their capacity to induce proliferation of NK cells and CD8 T cells within PBMCs upon digestion with recombinant human Matriptase.

The calculated amount of each molecule was digested with 3 μl recombinant human Matriptase (ALX-201-246-U250, Enzo) and filled up to 20 μl with Matriptase buffer (50 mM Tris, 50 mM NaCl, 0.01% Tween 20, pH 9.0). The digest was done for 2 hours at 37 °C. After digestion, the samples were filled up with RPMI+10% FBS+1% GlutaMax (assay medium) to have a final starting concentration of 100 nM.

Frozen PBMCs (Biomex) were thawed and immediately re-suspended in prewarmed assay medium. Cells were then centrifuged for 5 min at 350 g and washed once with prewarmed PBS. The CFSE stock solution was diluted 1:20 with prewarmed PBS to get a working solution with a concentration of 100 μM. 30 Mio cells were resuspended in 30 ml prewarmed PBS. 30 μl of the CFSE working solution was added to the cell suspension and the cells were mixed immediately and incubated for 15 min at 37 °C. Then, prewarmed assay medium was added to stop the reaction. The cells were spun down for 10 min at 400 g, resuspended in assay medium and incubated for 30 min at 37 °C. After the incubation the cells were washed once with prewarmed assay medium, counted and re-suspended in assay medium at 2 Mio cells per ml.

The CFSE labeled PBMCs were seeded into a 96 well round bottom plate (0.1 Mio cells per well) and the IL2 antibody was added and all wells were filled up to get a final volume of 200 μl. After incubation for 6 days at 37 °C, PBMCs were harvested. The cells were centrifuged for 4 min at 400 g and washed once with PBS. Live/dead stain was added in 25 μl PBS diluted 1:1000 in PBS) and incubated for 20 min at RT. Afterwards 150 μl FACS Buffer was added and the plate was centrifuged for 4 min at 400 g. Supernatant was removed and cells were washed again with 150 μl FACS Buffer. Then 25 μl per well of the antibody mix (BUV395 anti human CD3, PE anti human CD4, APC anti human CD8, PE/Cy7 anti human CD25, BV421 anti human CD56) was added to the cells. The cells were incubated for 30 min in the fridge. Afterwards the cells were washed twice with FACS buffer and resuspended in 150 μl FACS buffer. The analysis was performed using BD LSR Fortessa. NK cells were defined as CD3 negative and CD56 positive and CD8 T cells were defined as CD3 CD8 double positive.

CFSE labeled PBMCs were treated with TA PD1-IL2v molecules containing either a PQARK cleavage site or a YAARKGGI cleavage site and either a 38 mer or a 25 mer linker. As negative control, the respective non-cleavable TA PD1-IL2v molecules were included. As positive control, the respective unmasked PD1-IL2v molecule was included. After incubation for 6 days, PBMCs were analyzed by flow cytometry for activation marker upregulation and CFSE dilution was used as an indicator for proliferation.

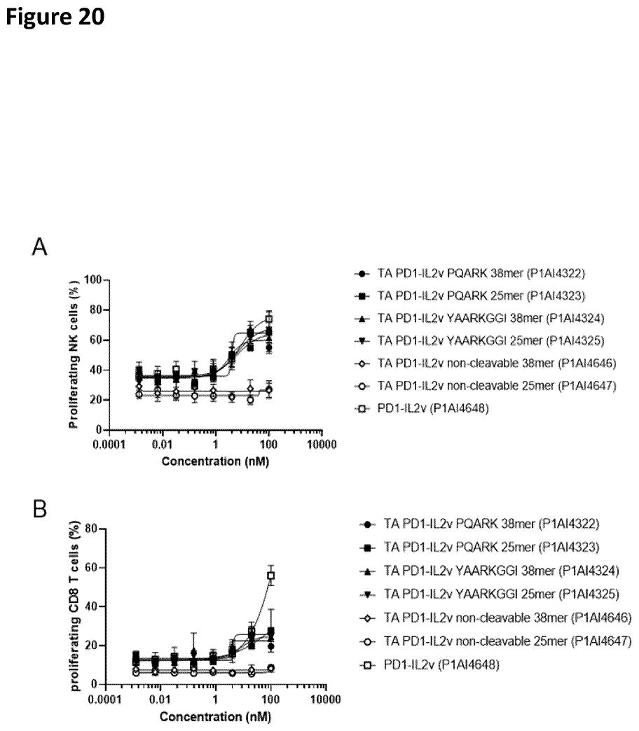

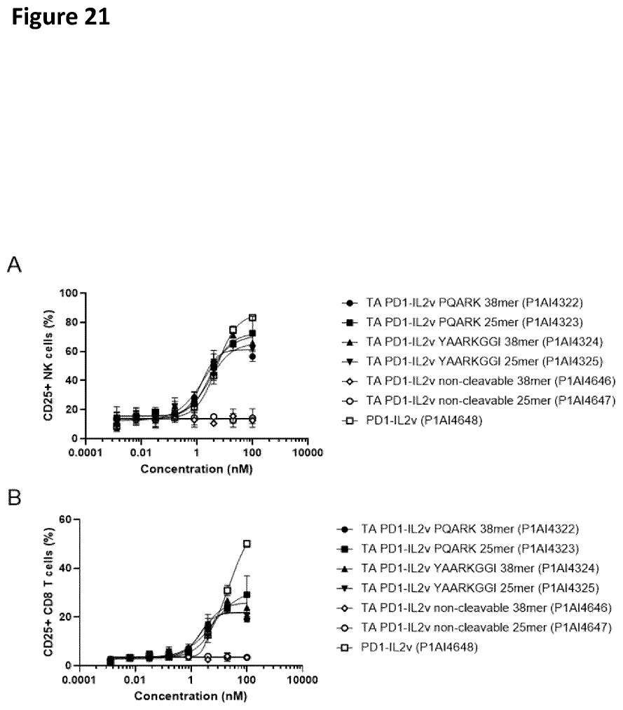

All predigested TA PD1-IL2v constructs containing a cleavage site were able to induce NK cell and CD8 T cell proliferation (FIG. 20A-B) and activation (FIG. 21A-B). No difference between the two tested linker length and the two cleavage sites could be observed in this assay and the pre-cleaved TA PD1-IL2v molecules have a comparable activity than the respective unmasked PD1-IL2v molecule. The two TA PD1-IL2v that did not contain a protease cleavage site did not induce any proliferation or activation of NK cells and CD8 T cells showing that the TA PD1-IL2v are only active upon protease cleavage.

### Example 9

### In Vivo Efficacy of Murinized TA-PD1-IL2v Immuno-Conjugates, in a Syngeneic Model of Mouse Tumor Cell Line-MCA205 Subcutaneous Syngeneic Model

The murinized TA-PD1-IL2v immuno-conjugates was tested in the mouse fibrosarcoma cell line MCA205, injected subcutaneously into Black 6-huPD1 transgenic mice.

The MCA205 fibrosarcoma carcinoma cells were originally obtained from Sigma Aldrich (Catalogue Number SCC173) and after expansion deposited in the Roche-Glycart internal cell bank. The tumor cell line was routinely cultured in DMEM containing 10% FCS (Gibco) at 37 °C in a water-saturated atmosphere at 5% CO2. Passage 9 was used for transplantation, at a viability of 97.9%, 1×10⁶ cells per animal were injected subcutaneously in 100 μl of RPMI cell culture medium (Gibco) into the flank of mice using a 1 ml tuberculin syringe (BD Biosciences, Germany).

Female Black 6-huPD1 mice, aged 8-10 weeks at the start of the experiment (bred at Charles Rivers, Lyon, France) were maintained under specific-pathogen-free condition with daily cycles of 12 h light/12 h darkness according to committed guidelines (GV-Solas; Felasa; TierschG). The experimental study protocol was reviewed and approved by local government (P 184/2020). After arrival, animals were maintained for one week to get accustomed to the new environment and for observation. Continuous health monitoring was carried out on a regular basis.

Mice were injected subcutaneously on study day 0 with 1×10⁶ of MCA205 cells, randomized and weighed. Ten days after the tumor cell injection (tumor volume>200 mm³), mice were injected i.v. with TA-PD1-IL-2v PQARK 25 mer cleavable linker or TA-PD1-IL-2v 25 mer non cleavable linker twice a week for one week. All mice were injected i.v. with 200 μl of the appropriate solution. The mice in the Vehicle group were injected with Histidine Buffer. To obtain the proper amount of immunoconjugate per 200 μl, the stock solutions were diluted with Histidine Buffer when necessary.

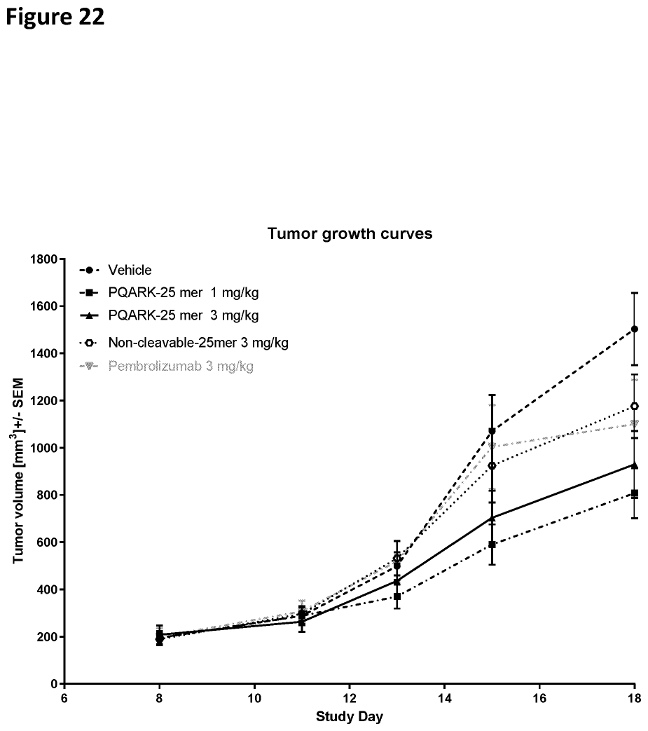

FIG. 22 shows that the TA-PD-IL2v PQARK 25 mer at both doses tested mediated superior efficacy in terms of tumor growth inhibition compared to vehicle, non-cleavable and Pembrolizumab Mabs single agent groups.

TABLE 5

| | Dose/ | Concentration |
|---|---|---|
| Compound | mouse | (mg/mL) |
| TA-PD1-IL2v PQARK | 20 μg | 3.1 |
| cleavable 25mer, | | (=stock solution) |
| P1AK3638-001 | | |
| TA-PD1-IL2v PQARK | 60 μg | 3.1 |
| cleavable 25mer, | | (=stock solution) |
| P1AK3638-001 | | |
| TA-PD1-IL2v non- | 60 μg | 8.70 |
| cleavable 25mer, | | (=stock solution) |
| P1AK3640-001 | | |
| Pembrolizumab | 60 μg | 25 |
| | | (=stock solution) |

### Example 10

### In Vivo Efficacy of Murine Surrogates for TA-PD1-IL2v Immuno-Conjugates, in a Syngeneic Model of Mouse Tumor Cell Line-GL261 Subcutaneous Syngeneic Model

The murine surrogates TA-PD1-IL2v immuno-conjugates was tested in the mouse glioblastoma cell line GL261, injected subcutaneously into Black 6 mice.

The GL261 glioblastoma cells were originally obtained from DSMZ (Germany) and after expansion deposited in the Roche-Glycart internal cell bank. The tumor cell line was routinely cultured in DMEM containing 10% FCS (Gibco) at 37 °C in a water-saturated atmosphere at 5% CO2. Passage 9 was used for transplantation, at a viability of 97.9%, 1×10⁶ cells per animal were injected subcutaneously in 100 μl of RPMI cell culture medium (Gibco) into the flank of mice using a 1 ml tuberculin syringe (BD Biosciences, Germany).

Female Black6 mice, aged 9-11 weeks at the start of the experiment (bred at Charles Rivers, Lyon, France) were maintained under specific-pathogen-free condition with daily cycles of 12 h light/12 h darkness according to committed guidelines (GV-Solas; Felasa; TierschG). The experimental study protocol was reviewed and approved by local government (P 184/2020). After arrival, animals were maintained for one week to get accustomed to the new environment and for observation. Continuous health monitoring was carried out on a regular basis.

Mice were injected subcutaneously on study day 0 with 1×10⁶ of GL261 cells, randomized and weighed. Ten days after the tumor cell injection (tumor volume>100 mm³), mice were injected i.v. with muTA-PD1-IL-2v PQARK 25 mer cleavable linker or muPD1-IgG twice a week for one week. All mice were injected i.v. with 200 μl of the appropriate solution. The mice in the Vehicle group were injected with Histidine Buffer. To obtain the proper amount of immune-conjugate per 200 μl, the stock solutions were diluted with Histidine Buffer when necessary.

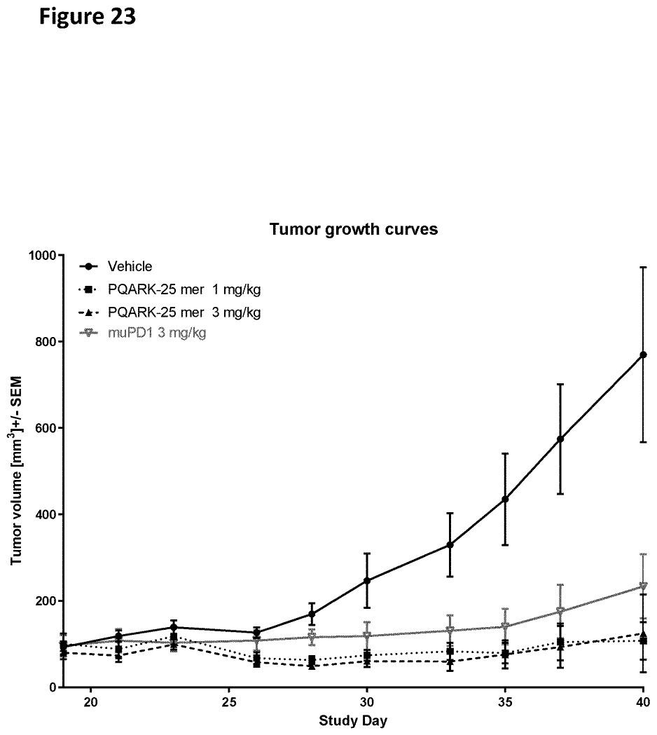

FIG. 23 shows that the muTA-PD-IL2v PQARK 25 mer at both doses tested mediated superior efficacy in terms of tumor growth inhibition compared to vehicle and muPD1 Mabs single agent groups.

TABLE 6

| | Dose/ | Concentration |
|---|---|---|
| Compound | mouse | (mg/mL) |
| muTA-PD1-IL2v PQARK | 20 μg | 3.4 |
| cleavable 25mer, | | (=stock solution) |
| P1AK3641 | | |
| muTA-PD1-IL2v PQARK | 60 μg | 3.4 |
| cleavable 25mer, | | (=stock solution) |
| P1AK3641 | | |
| muPD1-IgG | 60 μg | 3.03 |
| P1AD4006 | | (=stock solution) |

### Example 11

In Vivo Efficacy of TA-PD1-IL2v Immuno-Conjugate, in a Xenograft Model of Human Tumor PDX, in Combination with FOLR1-TCB Bispecific Mab-BC004 Breast Patient Derived Xenograft Model

TA-PD1-IL2v immune-conjugates were tested in combination with FOLR1-TCB bispecific antibody for its anti-tumoral efficacy in a xenograft model. The human TA-PD1-IL2v immune-conjugate was tested in the human breast BC004 patient derived cells subcutaneously injected into humanized NSG mice.

BC004 PDX material (human breast carcinoma) were originally obtained from OncoTest (Freiburg, Germany) and after in vivo expansion deposited in the Roche-Glycart internal cell bank. Tumor fragments were digested with Collagenase D and DNase I (Roche, Switzerland), and BC004 cells were used for transplantation. 1×10⁶ cells per animal were injected subcutaneously in 100 μl of RPMI cell culture medium (Gibco, Germany) into the flank of mice using a 1 ml tuberculin syringe (BD Biosciences, Germany).

Fully humanized NSG female mice (Roche-Glycart, Schlieren, Switzerland) were maintained under specific-pathogen-free condition with daily cycles of 12 h light/12 h darkness according to committed guidelines (GV-Solas; Felasa; TierschG). The experimental study protocol was reviewed and approved by local government (ZH184/2020). Continuous health monitoring was carried out on a regular basis.

Mice were injected subcutaneously on study day 0 with 1×10⁶ of BC004 cells, randomized and weighed. Thirty-two days after the tumor cell injection (tumor volume>150 mm³), mice were injected i.v. with the following immuno-cytokines: TA-PD1-IL2v-PQARK 25 mer cleavable 1 mg/kg, PD1-IL2v 0.1 mg/kg and Pembrolizumab 1 mg/kg in combination with FolR1-TCB or Vehicle once a week for four weeks. All mice were injected i.v. with 200 μl of the appropriate solution. The mice in the Vehicle group were injected with Histidine Buffer and the treatment groups with the different constructs. To obtain the proper amount of immuno-conjugate per 200 μl, the stock solutions were diluted with Histidine Buffer when necessary. Tumor growth measurements were evaluated with a caliper three times a week and plotted with GrahPad Prism software as volume in mm³+/−SEM.

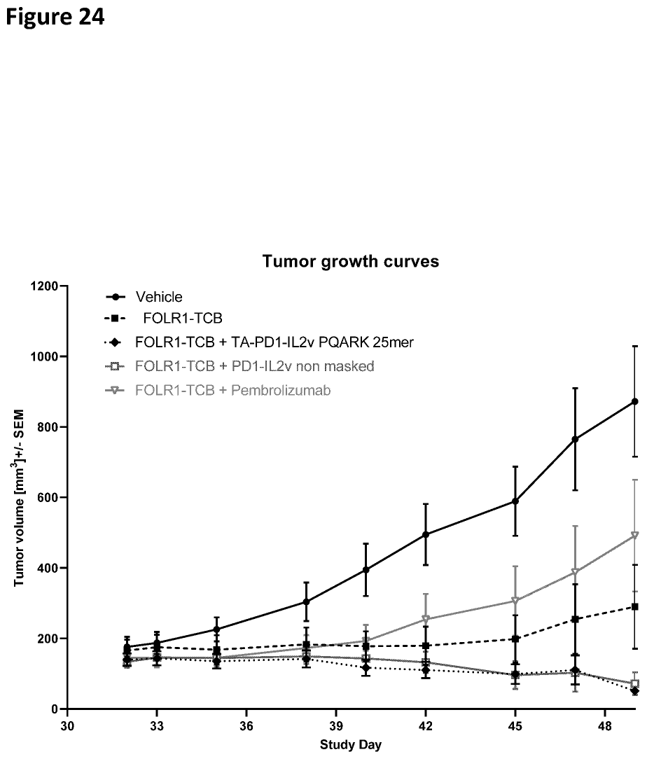

FIG. 24 shows that the combination FOLR1-TCB 0.3 mg/kg+TA-PD1-IL2v PQARK cleavable linker 1 mg/kg Mabs mediated superior efficacy in terms of tumor growth inhibition compared to FOLR1-TCB+Pembrolizumab 1 mg/kg. The benefit on efficacy showed similar tumor growth inhibition to the combination of FOLR1-TCB 0.3 mg/kg+PD1-IL2v unmasked 0.1 mg/kg Mabs.

TABLE 7

| | | Concentration |
|---|---|---|
| Compound | Dose | (mg/mL) |
| FOLR1-TCB | 6 μg | 3.47 |
| P1AK1120 | | (=stock solution) |
| TA-PD1-IL2v PQARK | 20 μg | 2.50 |
| cleavable | | (=stock solution) |
| P1AI4323 | | |
| PD1-IL2v | 2 μg | 1 |
| P1AA7146 | | (=stock solution) |
| Pembrolizumab | 20 μg | 25 |
| ID248 | | (=stock solution) |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A protease-activatable interleukin-2 (IL-2) polypeptide comprising (i) an IL-2 polypeptide, (ii) a masking moiety and (iii) a linker comprising a first protease cleavage site, wherein the linker has a length of 20 to 45 amino acids, wherein the masking moiety is covalently attached to the IL-2 polypeptide through the linker, wherein the masking moiety is capable of binding to the IL-2 polypeptide thereby reversibly concealing the IL-2 polypeptide, wherein the masking moiety comprises a second protease cleavage site, wherein the masking moiety does not conceal the IL-2 polypeptide upon cleavage at the first and/or the second protease cleavage site.

2. The protease activatable IL-2 polypeptide of claim 1, wherein the linker has a length of 22 to 43 amino acids.

3. The protease activatable IL-2 polypeptide of claim 1 or 2, wherein the linker has a length of 25 to 38 amino acids.

4. The protease activatable IL-2 polypeptide of any of claims 1 to 3, wherein the linker has a length of 25 or 38 amino acids.

5. The protease-activatable IL-2 polypeptide of any of the preceding claims, wherein the masking moiety is covalently attached to the amino-terminus or carboxy-terminus of the interleukin-2 polypeptide through the linker.

6. The protease-activatable IL-2 polypeptide of any of the preceding claims, wherein the masking moiety is an IL-2 antagonist.

7. The protease-activatable IL-2 polypeptide of any of the preceding claims, wherein the masking moiety is an IL-2 antibody or an IL-2 receptor subunit.

8. The protease-activatable IL-2 polypeptide of claim 7, wherein the IL-2 antibody comprises a Fab molecule.

9. The protease-activatable IL-2 polypeptide of any of the preceding claims, wherein the masking moiety is an antibody derived from antibody MT204.

10. The protease-activatable IL-2 polypeptide of claim 8 or 9, wherein the Fab molecule is a single-chain Fab molecule.

11. The protease-activatable IL-2 polypeptide of claim 10, wherein the second protease cleavage site is located between the variable domain of the heavy chain (VH) and the variable domain of the light chain (VL) of the single-chain Fab molecule.

12. The protease-activatable IL-2 polypeptide of any of the preceding claims, wherein the first protease cleavage site and the second protease cleavage site each comprise at least one protease recognition sequence.

13. The protease-activatable IL-2 polypeptide of any of the preceding claims, wherein the protease recognition sequence of the first protease cleavage site and/or the protease recognition sequence of the second protease cleavage site is YAARKGGI according to SEQ ID NO:60 and/or PQARK according to SEQ ID NO:61.

14. The protease-activatable IL-2 polypeptide of any of the preceding, wherein the IL-2 polypeptide is a wild-type IL-2, preferably a human IL-2 according to SEQ ID NO: 62, or a mutant IL-2 polypeptide.

15. The protease-activatable IL-2 polypeptide of claim 14, wherein the mutant IL-2 polypeptide comprises any amino acid substitution selected from the group T3A, F42A, Y45A, L72G, C125A of human IL-2 according to SEQ ID NO:62.

16. The protease-activatable IL-2 polypeptide of claim 14 or 15, wherein the mutant IL-2 polypeptide comprises the amino acid substitutions F42A, Y45A and L72G of human IL-2 according to SEQ ID NO:62.

17. The protease-activatable IL-2 polypeptide of any of claims 14 to 16, wherein the mutant IL-2 polypeptide comprises the amino acid substitutions T3A, F42A, Y45A, L72G and C125A of human IL-2 according to SEQ ID NO:62.

18. The protease-activatable IL-2 polypeptide of any of claims 14 to 17, comprising the amino acid sequence of SEQ ID NO: 27, SEQ ID NO: 28, SEQ ID NO: 29 or SEQ ID NO: 30.

19. The protease-activatable IL-2 polypeptide of any one the preceding claims, wherein the IL-2 polypeptide is further attached to a non-IL-2 moiety.

20. The protease-activatable IL-2 polypeptide of claim 19, wherein the IL-2 polypeptide shares a carboxy-terminal peptide bond with the masking moiety and an amino-terminal peptide bond with the non-IL-2 moiety or wherein the IL-2 polypeptide shares an amino-terminal peptide bond with the masking moiety and an carboxy-terminal peptide bond with the non-IL-2 moiety.

21. The protease-activatable IL-2 polypeptide of claim 19 or 20, wherein the non-IL-2 moiety is an antigen binding moiety or an effector cell binding moiety.

22. An immunoconjugate comprising a protease-activatable IL-2 polypeptide of claims 1 to 18 and an antigen binding moiety and/or an effector cell binding moiety.

23. The immunoconjugate of claim 22, wherein said protease-activatable IL-2 polypeptide shares an amino- or carboxy-terminal peptide bond with the antigen binding moiety or the effector cell binding moiety.

24. The immunoconjugate of claim 22 or 23, wherein said immunoconjugate comprises a first and a second antigen binding moiety or a first and a second effector cell antigen binding moiety or an antigen binding moiety and an effector cell binding moiety.

25. The immunoconjugate of claim 24, (i) wherein said protease-activatable IL-2 polypeptide shares an amino- or carboxy-terminal peptide bond with said first antigen binding moiety and said second antigen binding moiety shares an amino- or carboxy-terminal peptide bond with either a) said protease-activatable IL-2 polypeptide or b) said first antigen binding moiety; (ii) wherein said protease-activatable IL-2 polypeptide shares an amino- or carboxy-terminal peptide bond with said first effector cell binding moiety and said second effector cell binding moiety shares an amino- or carboxy-terminal peptide bond with either a) said protease-activatable IL-2 polypeptide or b) said first effector cell binding moiety; (iii) wherein said protease-activatable IL-2 polypeptide shares an amino- or carboxy-terminal peptide bond with the antigen binding moiety and the effector cell binding moiety shares an amino- or carboxy-terminal peptide bond with either a) said protease-activatable IL-2 polypeptide or b) said antigen binding moiety; or (iv) wherein said protease-activatable IL-2 polypeptide shares an amino- or carboxy-terminal peptide bond with the effector cell binding moiety and the antigen binding moiety shares an amino- or carboxy-terminal peptide bond with either a) said protease-activatable IL-2 polypeptide or b) said effector cell binding moiety.

26. The protease-activatable IL-2 polypeptide of claim 21 or the immunoconjugate of any one of claims 22 to 25, wherein said antigen binding moiety or effector cell binding moiety is an antibody or an antibody fragment.

27. The protease-activatable IL-2 polypeptide of claim 21 or the immunoconjugate of any one of claims 22 to 25, wherein said antigen binding moiety and/or said effector cell binding moiety is selected from a Fab molecule and a scFv molecule.

28. The protease-activatable IL-2 polypeptide of claim 21 or the immunoconjugate of any one of claims 22 to 25, wherein said antigen binding moiety and/or said effector cell binding moiety is an immunoglobulin molecule, particularly an IgG molecule.

29. The protease-activatable IL-2 polypeptide of claim 21 or the immunoconjugate of any one of claims 22 to 28, wherein said antigen binding moiety is directed to an antigen presented on a tumor cell or in a tumor cell environment and/or wherein said effector cell binding moiety is directed to an effector cell present in a tumor cell environment in order to achieve cis-targeting.

30. The immunoconjugate of any of claims 22 to 29,
(i) wherein the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 23;
(ii) wherein the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 24;
(iii) wherein the immunoconjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 25; or
(iv) wherein the imm immunoconjugate onjugate comprises an amino acid sequence that is at least about 95%, 96%, 97%, 98%, 99% or 100% identical to the sequence of SEQ ID NO: 5, an amino acid sequence that is at least about 95%, 96%, 97%, 98%, 99% or 100% identical to the sequence of SEQ ID NO: 22, and an amino acid sequence that is at least about 95%, 96%, 97%, 98%, 99% or 100% identical to the sequence of SEQ ID NO: 26.

31. The immunoconjugate of any of the claims 22 to 30,
(i) wherein the immunoconjugate comprises an amino acid sequence according to SEQ ID NO: 5, an amino acid sequence according to SEQ ID NO: 22, and an amino acid sequence according to SEQ ID NO: 23;
(ii) wherein the immunoconjugate comprises an amino acid sequence according to SEQ ID NO: 5, an amino acid sequence according to SEQ ID NO: 22, and an amino acid sequence according to SEQ ID NO: 24;
(iii) wherein the immunoconjugate comprises an amino acid sequence according to SEQ ID NO: 5, an amino acid sequence according to SEQ ID NO: 22, and an amino acid sequence according to SEQ ID NO: 25; or
(iv) wherein the immunoconjugate comprises an amino acid sequence according to SEQ ID NO: 5, an amino acid sequence according to SEQ ID NO: 22, and an amino acid sequence according to SEQ ID NO: 26.

32. An isolated polynucleotide encoding the protease-activatable IL-2 polypeptide or immunoconjugate of any one of claims 1 to 27.

33. An expression vector comprising the polynucleotide of claim 32.

34. A host cell comprising the polynucleotide of claim 32 or the expression vector of claim 33.

35. A method of producing a protease-activatable IL-2 polypeptide or an immunoconjugate thereof, comprising culturing the host cell of claim 34 under conditions suitable for the expression of the protease-activatable IL-2 polypeptide or the immunoconjugate.

36. A protease-activatable IL-2 polypeptide or immunoconjugate produced by the method of claim 35.

37. A pharmaceutical composition comprising the protease-activatable IL-2 polypeptide or immunoconjugate of any one of claim 1 to 31 or 36 and a pharmaceutically acceptable carrier.

38. The protease-activatable IL-2 polypeptide or immunoconjugate of any one of claim 1 to 31 or 36 for use in the treatment of a disease in an individual in need thereof.

39. The protease-activatable IL-2 polypeptide or immunoconjugate of claim 38 wherein said disease is cancer.

40. Use of the protease-activatable IL-2 polypeptide or immunoconjugate of any one of claim 1 to 31 or 36 for manufacture of a medicament for treating a disease in an individual in need thereof.

41. A method of treating disease in an individual, comprising administering to said individual a therapeutically effective amount of a composition comprising the protease-activatable IL-2 polypeptide or immunoconjugate of any one of claim 1 to 31 or 36 in a pharmaceutically acceptable form.

42. The method of claim 41, wherein said disease is cancer.

43. A method of stimulating the immune system of an individual, comprising administering to said individual an effective amount of a composition comprising the protease-activatable IL-2 polypeptide or immunoconjugate of any one of claim 1 to 31 or 36 in a pharmaceutically acceptable form.

44. The invention as described hereinbefore.
